(19) United States
(12) Patent Application Publication
(10) Pub. No.: US 2004/0131599 A1
(43) Pub. Date: Jul. 8, 2004

(54) Fas ligand expressing hematopoietic cells for transplantation

(76) Inventors: Curt I Civin, Towson, MD (US); Daniel Drachman, Stevenson, MD (US); Katherine Whartenby, Baltimore, MD (US); Drew M Pardoll, Brookville, MD (US)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/471,881

(22) PCT Filed: Mar. 13, 2002
(86) PCT No.: PCT/US02/07861

(51) Int. Cl. A61K 48/00; C12N 5/08

(52) U.S. Cl. 424/93.21; 435/372

(57) ABSTRACT

The invention provides methods and compositions utilizing FasL armed donor graft cells to reduce or eliminate host allogeneic or xenogeneic graft rejection, and FasL armed host cells to reduce or eliminate graft versus host disease.

### CROSS REFERENCE TO RELATED APPLICATIONS

This application claims the benefit of U.S. Provisional Application No. 60/275,615, filed Mar. 13, 2001, the contents of which are specifically incorporated herein.

### 1. BACKGROUND OF THE INVENTION

Tissue and organ transplants save many lives threatened by disease and cancer each year. Allogeneic grafts (or allografts) from human donor skin, kidney, liver, pancreas and heart are now commonplace, and Xenogeneic grafts (or xenografts) from non-human mammalian donor organisms are also being studied for their potential use as a broadly available source of tissues and organs. A particularly medically useful type of transplantation is allogeneic bone marrow transplantation. Allogeneic bone marrow transplantation may be used remedy acquired defects in either the hematopoietic system or the immune system, since both types of cells develop from a common stem cell. Furthermore, allogeneic bone marrow transplantation provides a means of correcting inherited enzymatic deficiencies or other genetic defects by providing a self-renewing source of the particular enzyme or other gene product missing in the affected individual. Still further, allogeneic bone marrow transplantation may be used to treat bone marrow malignancies—i.e. leukemias. Typically, treatment of leukemia involves the use of chemotherapeutic agents which destroy both the patient's normal bone marrow stem cell populations and the leukemia cancer cell populations. Accordingly, allogeneic bone marrow transplantation must be used following chemotherapy to restore the normal red and white blood cell progenitor cell populations in the patient. As a result of these treatments, recipients of allogeneic bone marrow transplants often show prolonged and profound immunodeficiences which are a major cause of morbidity and mortality. For the treatment of other cancers not involving the patient's bone marrow stem cell populations, the patient's own bone marrow may be harvested prior to and reinfused following chemotherapy in what is called an autologous bone marrow transplant.

The long term success of allogeneic and xenogeneic grafts requires that the graft survives the host's immune surveillance of foreign antigens. It has long been recognized that the normally functioning immune system of the transplant recipient recognizes the transplanted organ as “non-self” tissue and thereafter mounts an immune response to the presence of the transplanted organ. Left unchecked, the immune response will generate a plurality of cells and proteins that will ultimately result in the loss of biological functioning or the death of the transplanted organ. Therefore the long term success of allogeneic and xenogeneic transplants requires that immune responses mounted by the recipient against the donor graft be suppressed or prevented.

In order to prevent host immune rejection of tissue and organ grafts, transplant recipients are typically treated with one or more cytotoxic agents in an effort to suppress the transplant recipient's immune response against the transplanted organ or tissue. For example, cyclosporin (cyclosporin A), a cyclic polypeptide consisting of 11 amino acid residues and produced by the fungus species Tolypocladium inflatum Gams, is currently the drug of choice for administration to the recipients of allogeneic kidney, liver, pancreas and heart (i.e., wherein donor and recipient are of the same species of mammals) transplants. However, administration of cyclosporin is not without drawbacks as the drug can cause kidney and liver toxicity as well as hypertension. Moreover, use of cyclosporin can lead to malignancies (such as lymphoma) and lead to opportunistic infection due to the “global” nature of the immunosuppression it induces in patients receiving long term treatment with the drug, i.e., the hosts normal protective immune response to pathogenic microorganisms is downregulated thereby increasing the risk of infections caused by these agents. Other drugs for the prevention of graft rejection include: FK-506 (which has a similar mode of action as cyclosporine and is thought to be as potent as cyclosporin in its immunosuppressive qualities while having fewer toxic side effects than cyclosporin); steroids, such as prednisone, methylprednisalone, and Azathioprine (an analog of 6-mercaptopurine) (which are non-specific immunosuppressive drugs used to prolong allograft survival in transplantation recipients); and monoclonal antibodies (such as OKT3 monoclonal antibodies which are directed against the CD3 antigen present on T-cells and which have also been employed as non-specific immunosuppressive therapeutic agents in allograft recipients).

Another strategy used to suppress host immune rejection of graft cells in total lymphoid irradiation (TLI), another form of non-specific immunosuppressive therapy that has been used clinically and experimentally to prolong allograft survival. The radiation exposure and treatment schedule for TLI were developed for the treatment of Hodgkin's disease and were subsequently found to be immunosuppressive. TLI induces production of the “global” immunosuppression mentioned above and has the same limitations of other global immunosuppressive therapies.

A particularly severe drawback of the immunosuppressive drug therapies is that they must be administered indefinitely to suppress allogeneic graft rejection, and tolerance to the foreign tissue does not develop. Moreover, the general non-specific suppression of recipient allograft rejection by drug or irradiation treatment carries the risk of increased susceptibility to infection and malignancy (see DeMeo & Ginns (2001) Annu Rev Med 52: 185-201; Ridzon & Onorato (1998) N Engl J Med 338: 1741-51). Accordingly, alternative means of preventing host rejection of allogeneic or xenogeneic grafts would be desirable.

### 2. SUMMARY OF THE INVENTION

In general, the invention provides a method for suppressing the immune response of a recipient mammal to a donor hematopoietic stem cell graft by expressing a recombinant FasL gene in the donor hematopoietic stem cell of the donor graft. Expression of the recombinant FasL gene in the donor hematopoietic stem cell of the donor graft suppresses the immune response of the recipient mammal to the donor graft.

Without wishing to limit the invention to a particular mechanism of action, the expression of the recombinant FasL gene in the donor graft cell generally results in the selective deletion of developing anti-donor T cells of the recipient mammal. This selective deletion of developing anti-donor T cells in the graft recipient mammal thereby blocks development of an allogenic immune response to the graft, thereby preventing rejection of the transplanted cells. The method of the invention may be used in both allogeneic and xenogeneic applications, particularly allogeneic hematopoietic stem cell grafts and xenogeneic hematopoietic stem cell grafts.

In a preferred method of the invention, expression of the recombinant FasL gene in the donor hematopoietic cell is achieved by transducing the donor hematopoietic cell with a FasL gene expression vector. The FasL gene is preferrably derived from a mammalian organism, and may be any of a number of known or readily isolated FasL genes, such a FasL gene that hybridizes under stringent conditions to the FasL nucleic acid sequence shown in FIG. 8A. Preferably, the FasL gene expression vector includes a nucleotide sequence that encodes the polypeptide sequence shown in FIG. 8B. Still more preferably, the FasL gene expression vector encodes a non-cleavable form of FasL. Examples of non-cleavable forms of FasL include the naturally occurring FasL variant shown in FIG. 9B. Alternatively, the non-cleavable form of FasL may be a genetically engineered deletion mutant. The recombinant FasL gene may be expressed from a donor hematopoietic cell chromosome by integrating a FasL expression vector or by activating expression of the endogenous FasL gene by inserting an appropriate promoter by homologous recombination. More preferably, the recombinant FasL gene is expressed from a retroviral expression vector or a lentiviral expression vector.

In particularly preferred applications, the method of the invention further includes providing a donor dendritic cell expressing a recombinant FasL gene in combination with the FasL-expressing stem cell. Methods for expressing the recombinant FasL gene in the donor dendritic cell by transducing the donor graft cell with a FasL gene expression vector, are known in the art and are analogous to those used to effect FasL expression in the hematopoietic stem cell.

The invention further provides a recombinant donor hematopoietic stem cells expressing a recombinant FasL gene, and preferably, a non-cleavable FasL gene. Still more preferably, the recombinant donor hematopoietic stem cells further contains a dominant negative FasL signalling pathway component, such as a dominant negative FADD, which can be used to control the activity of the FasL in the donor graft hematopoietic stem cells and/or donor dendritic cells. In particularly prefered embodiments, the dominant negative FADD is a deletion mutant, for example a truncated FADD (see e.g. Wu et al. (2001) Cell Immunol 208: 137-47).

The invention further provides methods for increasing tolerance to a solid organ donor graft in a subject by also administering to the subject a recombinant hematopoietic cell graft from the donor organism expressing a FasL gene just prior to or in conjunction with the donor solid organ graft. The recombinant hematopoietic cell graft expressing the recombinant FasL gene from the donor organism increases tolerance to the donor solid organ graft, e.g. an allograft, in the subject. Suitable donor hematopoietic cell grafts for use in this aspect of the invention include dendritic cells and/or hematopoietic stem cells. This aspect of the invention can be used in conjunction with any solid organ allograft, e.g., cardiac allografts, kidney allografts, and liver allografts. In certain preferred embodiments, this solid organ allograft/FasL+ hematopoietic cell transplant therapy is further accompanied by treatment with an agent that promotes a co-stimulatory blockade and thereby interferes with immune rejection via a second mechanism. Examples of agents that promote a co-stimulatory blockade include anti-CD40 antibody and CTLA4-Ig.

The invention still further provides for methods and compositions useful for suppressing graft versus host disease in a host organism receiving a donor allograft by also administering to the recipient host organism an autologous hematopoietic cell graft expressing a recombinant FasL gene. The recombinant FasL+ autologous hematopoietic cell graft is preferably a hematopoietic stem cell or a dendritic cell.

### 3. BRIEF DESCRIPTION OF THE FIGURES

FIG. 1 depicts the Fas/FasL Pathway.

FIG. 2 shows that transient expression of plasmid vectors containing human FasL (FasL) causes apoptosis of Jurkat (human T lymphoid; Fas⁺) cells.

FIG. 3A shows that A20 (murine B lymphoid; Fas+) cells are protected from Fas-mediated apoptosis by transfection of dnFADD cDNA.

FIG. 3B shows that transduced FasL+dnFADD+A20 cells kill untransduced A20 cells.

FIG. 4A depicts certain retroviral expression vectors used to express FasL.

FIG. 4B depicts certain lentiviral vectors used to express FasL.

FIG. 5A shows that PG13/MGIN2-FasL+ cells express high levels of GFP.

FIG. 5B shows that PG13/MGIN2-FaSL+ cells induce apoptosis of Jurkat cells.

FIGS. 6A-B shows that transfection of the delFasL-GFP fusion cDNA results in membrane expressed GFP.

FIG. 6C shows that transfection delFasL retains the functionality of FasL.

FIG. 7A shows that administration of FasL+ Balb/c antigen presenting cells (APQ) reduces the numbers of specific T cells, in vivo in a transgenic model.

FIG. 7B shows that administration of FasL+ Balb/c antigen presenting cells (APC) reduces the proliferation of specific T cells, in vivo in a transgenic model.

FIG. 8A shows the nucleic acid sequence of a wild-type FasL gene (SEQ ID No. 1; GenBank U11821).

FIG. 8B shows the polypeptide sequence of a wild-type FasL polypeptide (SEQ ID No. 2; GenBank AAC50124).

FIG. 9A shows the nucleic acid sequence of a naturally occurring non-cleaved human Fas ligand expressed only in membrane bound form (SEQ ID No.3; Gen Bank AF288573).

FIG. 9B shows the polypeptide sequence of a naturally occurring non-cleaved human Fas ligand expressed only in membrane bound form (SEQ ID No.4; Gen Bank AAG60017.1).

FIG. 10 provides a diagrammatic representation of factors influencing the intensity of host versus graft reaction.

FIG. 11 shows that FasL-transduced donor APC and stem cells mights selectively injure recipient anti-allogeneic T cells.

FIG. 12 shows that 2C and Jurkat T cells are killed by FasL expressing cells.

FIG. 13A shows that FasL+ DCs decrease an allogeneic MLR.

FIG. 13B shows that FasL expression is required on the allogeneic APCs for full inhibition of MLR.

FIG. 14A shows that transduction of murine HSC with FasL does not affect their hematopoietic colony forming capacity.

FIG. 14B shows that colony forming capacity of human CD34+ stem/progenitor cells is not affected by soluble FasL exposure.

FIG. 14C shows that colony forming capacity of NOD/SCID repopulating cells is not decreased by sFasL exposure.

FIG. 15 shows that human CD34+ cells express FLIP.

FIG. 16A shows that in vivo treatment with FasL expressing dendritic cells decreases anti-allongeneic immune responsiveness.

FIG. 16B shows that FasL+ dendritic cells pre-tolerize recipients and enhance engraftment.

FIG. 16C shows the FACS plots of selected samples shown in the above graphs.

FIG. 17 shows that FasL modification of hematopoietic stem cells increases engraftment.

FIG. 18 shows in vivo treatment with FasL-transduced DCs enhanced allo engraftment.

FIG. 19 shows that FasL did not inhibit generation of CFCs from HSCs.

FIG. 20 shows that CD34⁺ and CD34⁺/CD38⁻ cells may be protected from Fas-mediated apoptosis by high levels of FLIP.

FIG. 21 shows that mice transplanted with FasL⁺ allo HSCs had enhanced engraftment.

FIG. 22 shows that mice transplanted with FasL⁺ HSCs did not have diminished numbers of BM CFCs.

FIG. 23 shows that mice transplanted with FasL⁺ HSCs did not have hepatocellular injury or enhanced hepatic inflammation, and the mouse cells retained immune responsiveness to a third party alloAg.

FIG. 24 shows that mice transplanted with syngeneic FasL⁺ HSCs responded to an antigenic infectious challenge.

FIG. 25 shows that transplanted FasL⁺ HSCs generated FasL⁺ DCs in vivo.

### 4. DETAILED DESCRIPTION OF THE INVENTION

4.1. General

The present invention provides methods and reagents to reduce or prevent allogeneic or xenogeneic graft rejection in hematopoietic and solid organ transplantation, and to reduce rejection of modified autologous cells (such as cells that have had a wild-type gene introduced). In preferred embodiments, the invention provides long-lasting immunosuppressive (tolerizing) effects on the host which prevent graft rejection by the host immune system. In addition, the invention can be used to reduce or prevent Graft Versus Host Disease (GVHD). In general, the invention relates to uses of the Fas/FasL pathway to eliminate activated host T cells which would otherwise lead to graft rejection. In a particular application of the invention, a FasL (Fas Ligand) gene is introduced into a donor graft cell, such as a dendritic cell or a stem cell, so that the donor graft is capable of expressing a recombinant FasL gene product. Without limiting the invention to a particular mechanism of action, expression of the recombinant FasL gene in the donor graft cell results in the suppression of the immune response of the recipient mammal to the donor graft by causing the selective deletion of developing anti-donor T cells of the recipient mammal.

In one embodiment, the invention provides a means of reducing the host's capacity to reject donor lymph-hematopoietic cells in allogeneic blood and marrow transplantation (BMT). In this application, the FasL gene is introduced into a donor graft cell to decrease the host cellular response specifically against allogeneic donor cells (Host versus Graft rejection; HVG). FasL is introduced into dendritic cells (DC) and/or lymphohematopoietic stem-progenitor cells (HSQ) by transduction with onco-retroviral and lentiviral vectors. The DC “armed” by gene transduction to express FasL (FasL⁺) kill cognate activated T cells. In preferred embodiments, transduced FasL⁺ DC are administered in vivo to decrease HVG in allogeneic BMT. In another aspect, FasL transduced HSC engineered to express FasL are used to kill attacking host T cells and thus selectively reduce HVG (mediated by FasL-susceptible cells, such as, activated T cells). The FasL⁺ HSC from the donor may be used to partially or completely and specifically tolerize hosts to the donor graft. In preferred embodiments, the use of FasL⁺ DC and/or HSC from the donor allows the treating physician to reduce or eliminate the nonspecific radiopharmacologic immunosuppression, currently given to patients undergoing allogeneic BMT to prevent HVG. This application of the invention may also be used in xenogeneic BMT applications.

In a second embodiment, the invention provides means to selectively reduce a host's capacity to reject transplants of allogencic solid organs (including cells and tissue from these organs and also pluripotent stem cells capable of developing into a variety of tissues and organs), such as kidney, pancreas, heart, liver, lung, and brain. Immunity against donor histocompatibility (rejection) antigens is reduced specifically by administration of FasL+ DC and/or HSC from the organ donor (or from someone with the same, or some of the same, histocompatibility antigens as the donor). This application of the invention may also be applied to xenotransplantation.

In a third embodiment, the invention provides means to selectively reduce the host's capacity to reject autologous cells that have been modified by an introduced or heterologous gene. In therapies for certain genetic diseases caused by a mutant form of a key gene, a wild-type (non-mutant) form of the gene is introduced into the host. One problem which reduces the efficacy of such gene therapy is that the patient may mount an immune response against the wild-type protein or some other component of the vector—both of which the patient's immune system may see as “non-self”. Accordingly, the instant invention may be applied to this problem of autologous cell gene therapy by introducing an heterologous FasL gene into the genetically engineered autologous cell. This aspect of the invention to prevent host immune rejection of gene-transduced donor cells may be used in xenotransplantation or in combination with methods for reducing or preventing FasL-mediated allogeneic immunity.

A fourth aspect of the invention provides methods for selectively reducing or preventing a donor graft from immunologically attacking host cells (i.e. Graft versus Host Disease; GVHD). GVHD can occur when donor graft cells mount a rejection response against the host. This response, called a graft-versus-host reaction, can injure the host and cause GVHD. Such a graft-versus-host reaction typically arises after extreme injury to the host immune system, such as after chemotherapeutic or radiological treatment to destroy host bone marrow stem cell populations. In this application of the invention, armed FasL⁺ host cells (e.g. autologous dendritic or stem cell populations) are prepared and administered to the transplant recipient, with or after the allogeneic BMT. The FasL⁺ “armed” host cells destroy anti-allogeneic donor graft cells that attack them, and thus deplete the clones of GVHD-mediating donor cells. In additional aspect of this embodiment of the invention, the donor graft may be selectively depleted of donor-reactive immune cells that could mediate GVHD by contacting the donor graft cells, e.g. hematopoietic donor graft cells, with “armed” FasL⁺ host cells “in vitro” prior to transplantation of the donor graft into the patient. This aspect of the invention, in which a donor graft is treated to prevent GVHD, may also be applied to xenotransplantation.

In certain applications of the invention, donor graft dendritic cells, a specialized type of antigen presenting cell, are “armed” with an heterologous or recombinant FasL gene which is capable of constitutive or regulatable expression of a FasL gene product. Since dendritic cells are normally involved in physiologically tolerizing T cells, the FasL expressing donor graft dendritic cells of the invention are particularly well adapted to reaching and effecting tolerance in host T cells which would otherwise mediate immune rejection of the donor graft.

In preferred embodiments of the invention, donor graft stem cells are armed with the heterologous or recombinant FasL expression system. A particular advantage of this embodiment of the invention is that such stem cell populations (e.g. hematopoietic stem cells) persistently remain in the host because they are long-lived and possibly even immortal. Furthermore, such FasL armed stem cell populations produce massive numbers of progeny cells, and, accordingly, the use of FasL armed stem cells is more potent and longer-lasting than the use of other cell types. In certain embodiments, the invention employs both FasL armed dendritic donor graft cells to effect acute short-term host tolerance and FasL armed donor graft stem cells to effect long term and persistent host tolerance to the graft. In other preferred embodiments utilizing FasL armed dendritic and stem cells alone or in combination, the potency of the FasL-mediated host tolerance is increased by co-expressing CD8 or another molecule capable of binding to or recruiting a protein or other structure (e.g. CD25) expressed by activated T cells. In still other embodiments, the population of FasL armed graft cells is engineered so as to be susceptible to destruction after the desired long-term tolerance is achieved. For example, the FasL armed cells may be engineered so as to be capable of expressing a viral thymidine kinase gene. After the desired clinical effect of long-term host tolerance is achieved, the armed cells may be destroyed by treating the patient with ganciclovir, which selectively kills thymidine kinase expressing cells.

In other embodiments, the invention provides compositions and methods useful in preventing undesirable effects of long-term expression of FasL by the armed donor graft cell. In one aspect of this embodiment, the invention provides recombinant heterologous FasL genes and gene products which are modified from that of the naturally occurring endogenous FasL gene. In a particularly preferred embodiments of this aspect of the invention, the recombinant FasL gene constructs are modified to produce a non-cleavable form of FasL. This aspect of the invention is particularly useful in preventing injury of certain cell types, such as liver cells, in the host patient resulting from the release of biologically active fragments of FasL, which may be released from FasL armed donor graft cells of the invention. In another aspect of this embodiment, the invention provides means of regulating expression of FasL by the armed FasL donor graft cell. For example, the donor graft cell can be engineered so that expression of FasL is dependent upon an inducible or repressible promoter. Alternatively, a dominant negative component of the Fas pathway such as a dominant negative mutant form of the Fas associated death domain (FADD) (e.g. truncated FADD—see e.g. Wu et al. (2001) Cell Immunol 208: 137-47). or another molecule capable of blocking the Fas pathway, such as the Fas-associated death domain-like interleukin-1beta-converting enzyme-inhibitory protein (FLIP), may be introduced into the FasL armed cells of the invention to protect the armed cell from FasL-induced death.

4.2. Definitions

For convenience, the meaning of certain terms and phrases employed in the specification, examples, and appended claims are provided below.

The term “aberrant activity”, as applied to an activity of a polypeptide refers to an activity which differs from the activity of the wild-type or native polypeptide or which differs from the activity of the polypeptide in a healthy subject. An activity of a polypeptide can be aberrant because it is stronger than the activity of its native counterpart. Alternatively, an activity can be aberrant because it is weaker or absent relative to the activity of its native counterpart. An aberrant activity can also be a change in an activity. For example, an aberrant polypeptide can interact with a different target peptide. A cell can have an aberrant polypeptide activity due to overexpression or underexpression of the gene encoding the polypeptide.

The term “agonist”, as used herein, is meant to refer to an agent that mimics or upregulates (e.g. potentiates or supplements) a bioactivity. A polypeptide agonist can be a wild-type protein or derivative thereof having at least one bioactivity of the wild-type polypeptide. A polypeptide therapeutic can also be a compound that upregulates expression of a polypeptide-encoding gene or which increases at least one bioactivity of a polypeptide. An agonist can also be a compound which increases the interaction of a polypeptide with another molecule, thereby promoting.

The term “allele”, which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions, and insertions of nucleotides. Frequently occurring sequence variations include transition mutations (i.e. purine to purine substitutions and pyrimidine to pyrimidine substitutions, e.g. A to G or C to T), transversion mutations (i.e. purine to pyrimidine and pyrimidine to purine substitutions, e.g. A to T or C to G), and alteration in repetitive DNA sequences (e.g. expansions and contractions of trinucleotide repeat and other tandem repeat sequences). An allele of a gene can also be a form of a gene containing a mutation. The term “allelic variant of a polymorphic region of a FasL gene” refers to a region of a FasL gene having one or several nucleotide sequence differences found in that region of the gene in certain individuals.

“Antagonist” as used herein is meant to refer to an agent that downregulates (e.g. suppresses or inhibits) at least one bioactivity. An antagonist can be a compound which inhibits or decreases the interaction between a protein and another molecule, e.g., a FasL ligand and a FasL receptor. An antagonist can also be a compound that down-regulates expression of a gene or which reduces the amount of gene product protein present. The FasL antagonist can be a dominant negative form of a FasL polypeptide, e.g., a form of a FasL polypeptide which is capable of interacting with a target peptide. An antagonist can also be a compound that interferes with a protein-dependent signal transduction pathway—e.g. a dominant negative FADD which blocks downstream FasL signaling. The FasL antagonist can also be a nucleic acid encoding a dominant negative form of a FasL polypeptide, a FasL antisense nucleic acid, or a ribozyme capable of interacting specifically with a FasL RNA. Yet other FasL antagonists are molecules which bind to a FasL polypeptide and inhibit its action. Such molecules include peptides, e.g., forms of FasL target peptides which do not have biological activity, and which inhibit binding to FasL target molecules, such as the FasL receptor.

The term “antibody” as used herein is intended to include whole antibodies, e.g., of any isotype (IgG, IgA, IgM, IgE, etc), and includes fragments thereof which are also specifically reactive with a vertebrate, e.g., mammalian, protein. Antibodies can be fragmented using conventional techniques and the fragments screened for utility in the same manner as described above for whole antibodies. Thus, the term includes segments of proteolytically-cleaved or recombinantly-prepared portions of an antibody molecule that are capable of selectively reacting with a certain protein. Nonlimiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The subject invention includes polyclonal, monoclonal, or other purified preparations of antibodies and recombinant antibodies.

A disease, disorder, or condition “associated with” or “characterized by” an aberrant expression of a nucleic acid refers to a disease, disorder, or condition in a subject which is caused by, contributed to by, or causative of an aberrant level of expression of a nucleic acid.

As used herein the term “bioactive fragment of a FasL polypeptide” refers to a fragment of a full-length FasL polypeptide, wherein the fragment specifically mimics or antagonizes the activity of a wild-type FasL polypeptide. The bioactive fragment preferably is a fragment capable of interacting with a FasL receptor.

“Biological activity” or “bioactivity” or “activity” or “biological function”, which are used interchangeably, for the purposes herein means an effector or antigenic function that is directly or indirectly performed by a polypeptide (whether in its native or denatured conformation), or by any subsequence thereof. Biological activities include binding to a target peptide. A FasL bioactivity can be modulated by directly affecting a FasL polypeptide. Alternatively, a FasL bioactivity can be modulated by modulating the level of a FasL polypeptide, such as by modulating expression of a FasL gene.

The term “biomarker” refers a biological molecule, e.g., a nucleic acid, peptide, hormone, etc., whose presence or concentration can be detected and correlated with a known condition, such as a disease state.

“Cells”, “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A “chimeric polypeptide” or “fusion polypeptide” is a fusion of a first amino acid sequence encoding one of the subject polypeptides with a second amino acid sequence defining a domain (e.g. polypeptide portion) foreign to and not substantially homologous with any domain of the polypeptide. A chimeric polypeptide may present a foreign domain which is found (albeit in a different polypeptide) in an organism which also expresses the first polypeptide, or it may be an “interspecies”, “intergenic”, etc. fusion of polypeptide structures expressed by different kinds of organisms. In general, a fusion polypeptide can be represented by the general formula X-polypeptide-Y, wherein “polypeptide” represents a portion or all of a protein of interest and X and Y are independently absent or represent amino acid sequences which are not related to the protein sequence in an organism, including naturally occurring mutants.

As used herein, the term “co-stimulatory blockade” refers to any of a number of known means for effecting immunosuppression, e.g. in an allograft, by interfering with co-stimulatory signals between antigen-presenting and -responding cells. For example, T-cell activation, which is an essential feature of graft rejection, requires a first signal provided by T-cell receptor (TCR) ligation and a second signal provided by engagement of co-stimulatory molecules with their respective ligands on antigen-presenting cells. The coordinated triggering of these two independent signalling systems ensures the full T-cell activation, including proliferation and acquisition of effector function. TCR occupancy in the absence of co-stimulatory signals leads to a sustained loss of antigen responsiveness called clonal anergy, which could be of major importance in transplantation. In vivo, co-stimulation blockade allow for long-term allograft survival in transplantation models. Examples of means for interfering with signals between antigen-presenting and -responding cells include agents that bind to and interfere with the functioning of immune cell surface proteins that mediate cell-cell-specific immune cell interaction. Examples include monoclonal antibodies (mAbs) directed against CD antigens including mAbs against: CD28, CD40, CD80, CD86, and CD 154. Other agents for effecting co-stimulatory blockade include CTLA4-Ig.

A “delivery complex” shall mean a targeting means (e.g. a molecule that results in higher affinity binding of a gene, protein, polypeptide or peptide to a target cell surface and/or increased cellular or nuclear uptake by a target cell). Examples of targeting means include: sterols (e.g. cholesterol), lipids (e.g. a cationic lipid, virosome or liposome), viruses (e.g. adenovirus, adeno-associated virus, and retrovirus) or target cell specific binding agents (e.g. ligands recognized by target cell specific receptors). Preferred complexes are sufficiently stable in vivo to prevent significant uncoupling prior to internalization by the target cell. However, the complex is cleavable under appropriate conditions within the cell so that the gene, protein, polypeptide or peptide is released in a functional form.

The term “dendritic cell” refers to any of various accessory cells that serve as antigen-presenting cells (APCs) in the induction of an immune response. As used herein, the term “dendritic cell” includes both interdigitating dendritic cells which are present in the interstitium of most organs and are abundant in T cell-rich areas of the lymph nodes and spleen, as well as throughout the epidermis of the skin, where they are also referred to as Langerhans cells. The interdigitating dendritic cells arise from marrow precursor cells and are related in lineage to mononuclear phagocytes.

As is well known, genes may exist in single or multiple copies within the genome of an individual. Such duplicate genes may be identical or may have certain modifications, including nucleotide substitutions, additions or deletions, which all still code for polypeptides having substantially the same activity. For example, the term “DNA sequence encoding a FasL polypeptide” may thus refer to one or more FasL genes within a particular individual. Moreover, certain differences in nucleotide sequences may exist between individual organisms, which are called alleles. Such allelic differences may or may not result in differences in amino acid sequence of the encoded polypeptide yet still encode a polypeptide with the same biological activity.

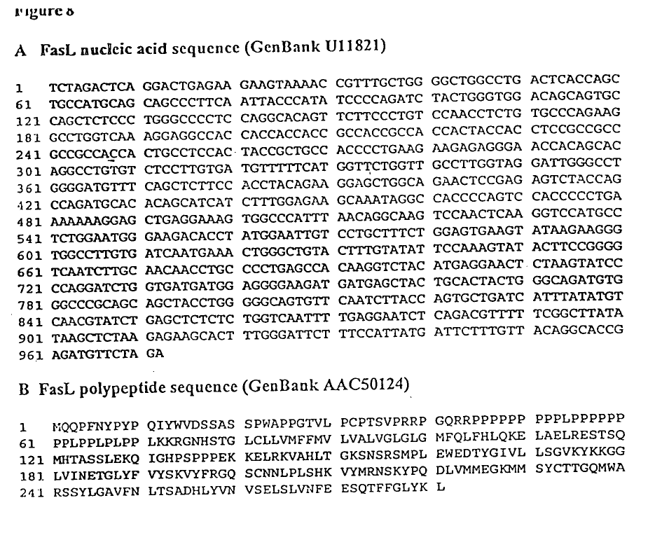

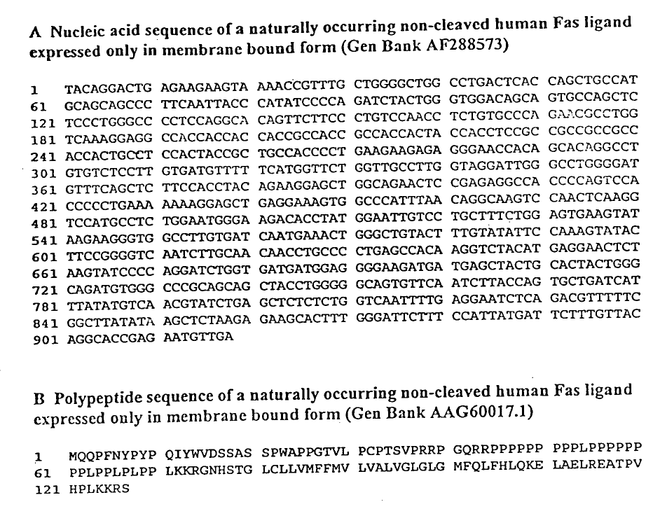

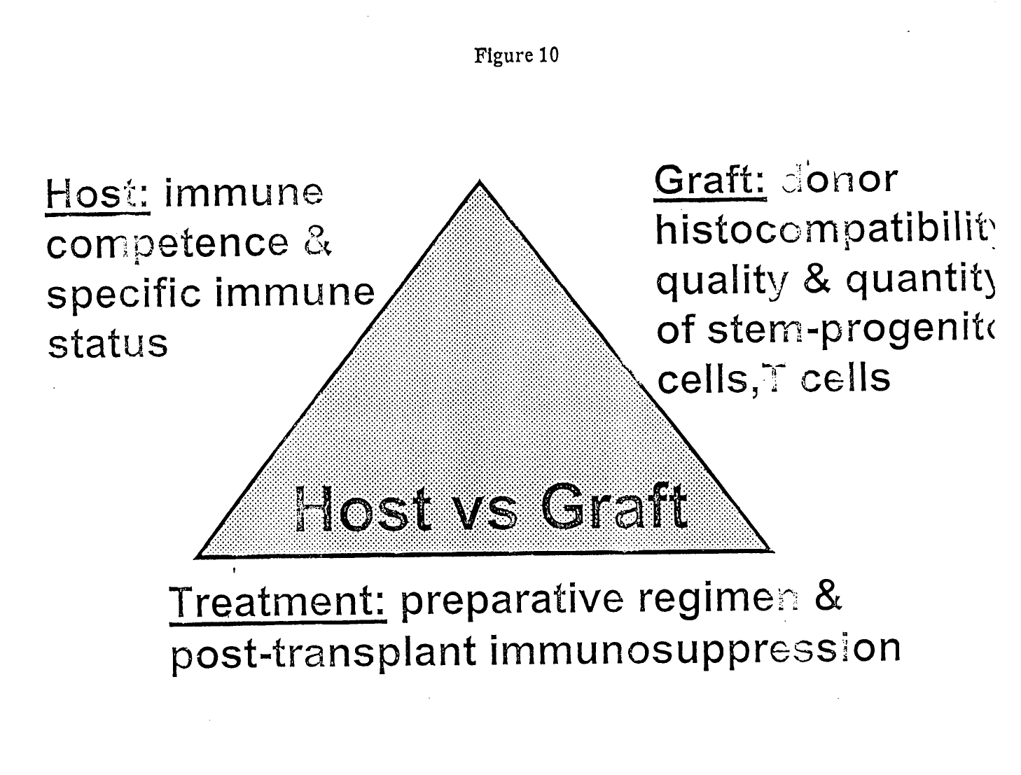

The term “equivalent” is understood to include nucleotide sequences encoding functionally equivalent polypeptides. Equivalent nucleotide sequences will include sequences that differ by one or more nucleotide substitutions, additions or deletions, such as allelic variants; and will, therefore, include sequences that differ from the nucleotide sequence of the nucleic acids shown in, for example, FIGS. 8A and 9A, due to the degeneracy of the genetic code.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are identical at that position. A degree of homology or similarity or identity between nucleic acid sequences is a function of the number of identical or matching nucleotides at positions shared by the nucleic acid sequences. A degree of identity of amino acid sequences is a function of the number of identical amino acids at positions shared by the amino acid sequences. A degree of homology or similarity of amino acid sequences is a function of the number of amino acids, i.e. structurally related, at positions shared by the amino acid sequences. An “unrelated” or “non-homologous” sequence shares less than 40% identity, though preferably less than 25% identity, with one of the sequences of the present invention.

The term “FasL binding partner” or “FasL BP” refers to various cell proteins which bind to a FasL protein.

The term “interact” as used herein is meant to include detectable relationships or association (e.g. biochemical interactions) between molecules, such as interaction between protein-protein, protein-nucleic acid, nucleic acid-nucleic acid, and protein-small molecule or nucleic acid-small molecule in nature.

The term “isolated” as used herein with respect to nucleic acids, such as DNA or RNA, refers to molecules separated from other DNAs, or RNAs, respectively, that are present in the natural source of the macromolecule. For example, an isolated nucleic acid encoding one of the subject polypeptides preferably includes no more than 10 kilobases (kb) of nucleic acid sequence which naturally immediately flanks the subject gene in genomic DNA, more preferably no more than 5 kb of such naturally occurring flanking sequences, and most preferably less than 1.5 kb of such naturally occurring flanking sequence. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

A “knock-in” transgenic animal refers to an animal that has had a modified gene introduced into its genome and the modified gene can be of exogenous or endogenous origin.

A “knock-out” transgenic animal refers to an animal in which there is partial or complete suppression of the expression of an endogenous gene (e.g, based on deletion of at least a portion of the gene, replacement of at least a portion of the gene with a second sequence, introduction of stop codons, the mutation of bases encoding critical amino acids, or the removal of an intron junction, etc.). In preferred embodiments, the “knock-out” gene locus corresponding to the modified endogenous gene no longer encodes a functional polypeptide activity and is said to be a “null” allele. Accordingly, knock-out transgenic animals of the present invention include those carrying one null gene mutation, as well as those carrying two null gene mutations.

A “knock-out construct” refers to a nucleic acid sequence that can be used to decrease or suppress expression of a protein encoded by endogenous DNA sequences in a cell. In a simple example, the knock-out construct is comprised of a gene with a deletion in a critical portion of the gene so that active protein cannot be expressed therefrom. Alternatively, a number of termination codons can be added to the native gene to cause early termination of the protein or an intron junction can be inactivated. In a typical knock-out construct, some portion of the gene is replaced with a selectable marker (such as the neo gene) so that the gene can be represented as follows: gene 5′/neo/gene 3′, where gene 5′ and gene 3′, refer to genomic or cDNA sequences which are, respectively, upstream and downstream relative to a portion of the gene and where neo refers to a neomycin resistance gene. In another knock-out construct, a second selectable marker is added in a flanking position so that the gene can be represented as: gene/neo/gene/TK, where TK is a thymidine kinase gene which can be added to either the gene 5′ or the gene 3′ sequence of the preceding construct and which further can be selected against (i.e. is a negative selectable marker) in appropriate media. This two-marker construct allows the selection of homologous recombination events, which removes the flanking TK marker, from non-homologous recombination events which typically retain the TK sequences. The gene deletion and/or replacement can be from the exons, introns, especially intron junctions, and/or the regulatory regions such as promoters.

The term “modulation” as used herein refers to both upregulation (i.e., activation or stimulation (e.g., by agonizing or potentiating)) and downregulation (i.e. inhibition or suppression (e.g., by antagonizing, decreasing or inhibiting)).

The term “mutated gene” refers to an allelic form of a gene, which is capable of altering the phenotype of a subject having the mutated gene relative to a subject which does not have the mutated gene. If a subject must be homozygous for this mutation to have an altered phenotype, the mutation is said to be recessive. If one copy of the mutated gene is sufficient to alter the genotype of the subject, the mutation is said to be dominant. If a subject has one copy of the mutated gene and has a phenotype that is intermediate between that of a homozygous and that of a heterozygous subject (for that gene), the mutation is said to be co-dominant.

The “non-human animals” of the invention include mammalians such as rodents, non-human primates, sheep, dog, cow, chickens, amphibians, reptiles, etc. Preferred non-human animals are selected from the rodent family including rat and mouse, most preferably mouse, though transgenic amphibians, such as members of the Xenopus genus, and transgenic chickens can also provide important tools for understanding and identifying agents which can affect, for example, embryogenesis and tissue formation. The term “chimeric animal” is used herein to refer to animals in which the recombinant gene is found, or in which the recombinant gene is expressed in some but not all cells of the animal. The term “tissue-specific chimeric animal” indicates that one of the recombinant genes of the invention is present and/or expressed or disrupted in some tissues but not others.

As used herein, the term “nucleic acid” refers to polynucleotides or oligonucleotides such as deoxyribonucleic acid (DNA), and, where appropriate, ribonucleic acid (RNA). The term should also be understood to include, as equivalents, analogs of either RNA or DNA made from nucleotide analogs and as applicable to the embodiment being described, single (sense or antisense) and double-stranded polynucleotides.

The term “nucleotide sequence complementary to the nucleotide sequence set forth in SEQ ID No. x” refers to the nucleotide sequence of the complementary strand of a nucleic acid strand having SEQ ID No. x. The term “complementary strand” is used herein interchangeably with the term “complement”. The complement of a nucleic acid strand can be the complement of a coding strand or the complement of a non-coding strand. When referring to double stranded nucleic acids, the complement of a nucleic acid having SEQ ID No. x refers to the complementary strand of the strand having SEQ ID No. x or to any nucleic acid having the nucleotide sequence of the complementary strand of SEQ ID No. x. When referring to a single stranded nucleic acid having the nucleotide sequence SEQ ID No. x, the complement of this nucleic acid is a nucleic acid having a nucleotide sequence which is complementary to that of SEQ ID No. x. The nucleotide sequences and complementary sequences thereof are always given in the 5′ to 3′ direction.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

Databases with individual sequences are described in Methods in Enzymology, ed. Doolittle, supra. Databases include Genbank, EMBL, and DNA Database of Japan (DDBJ).

Preferred nucleic acids have a sequence at least 70%, and more preferably 80% identical and more preferably 90% and even more preferably at least 95% identical to an nucleic acid sequence of a sequence shown in one of SEQ ID Nos. of the invention. Nucleic acids at least 90%, more preferably 95%, and most preferably at least about 98-99% identical with a nucleic sequence represented in one of the SEQ ID Nos. of the invention are of course also within the scope of the invention. In preferred embodiments, the nucleic acid is mammalian. In comparing a new nucleic acid with known sequences, several alignment tools are available. Examples include PileUp, which creates a multiple sequence alignment, and is described in Feng et al., J. Mol. Evol. (1987) 25:351-360. Another method, GAP, uses the alignment method of Needleman et al., J. Mol. Biol. (1970) 48:443-453. GAP is best suited for global alignment of sequences. A third method, BestFit, functions by inserting gaps to maximize the number of matches using the local homology algorithm of Smith and Waterman, Adv. Appl. Math. (1981) 2:482-489.

The term “polymorphism” refers to the coexistence of more than one form of a gene or portion (e.g., allelic variant) thereof. A portion of a gene of which there are at least two different forms, i.e., two different nucleotide sequences, is referred to as a “polymorphic region of a gene”. A polymorphic region can be a single nucleotide, the identity of which differs in different alleles. A polymorphic region can also be several nucleotides long.

A “polymorphic gene” refers to a gene having at least one polymorphic region.

As used herein, the term “promoter” means a DNA sequence that regulates expression of a selected DNA sequence operably linked to the promoter, and which effects expression of the selected DNA sequence in cells. The term encompasses “tissue specific” promoters, i.e. promoters, which effect expression of the selected DNA sequence only in specific cells (e.g. cells of a specific tissue). The term also covers so-called “leaky” promoters, which regulate expression of a selected DNA primarily in one tissue, but cause expression in other tissues as well. The term also encompasses non-tissue specific promoters and promoters that constitutively express or that are inducible (i.e. expression levels can be controlled).

The terms “protein”, “polypeptide” and “peptide” are used interchangeably herein when referring to a gene product.

The term “recombinant protein” refers to a polypeptide of the present invention which is produced by recombinant DNA techniques, wherein generally, DNA encoding a particular polypeptide is inserted into a suitable expression vector which is in turn used to transform a host cell to produce the heterologous protein. Moreover, the phrase “derived from”, with respect to a particular recombinant gene, is meant to include within the meaning of “recombinant protein” those proteins having an amino acid sequence of a particular native polypeptide, or an amino acid sequence similar thereto which is generated by mutations including substitutions and deletions (including truncation) of a naturally occurring form of the polypeptide.

“Small molecule” as used herein, is meant to refer to a composition, which has a molecular weight of less than about 5 kD and most preferably less than about 4 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays of the invention to identify compounds that modulate a bioactivity.

The term “stem cell” or “hematopoietic stem cell” is meant a pluripotent cell of the hematopoietic system capable of differentiating into cells of the lymphoid and myeloid lineages.

As used herein, the term “specifically hybridizes” or “specifically detects” refers to the ability of a nucleic acid molecule of the invention to hybridize to at least approximately 6, 12, 20, 30, 50, 100, 150, 200, 300, 350, 400 or 425 consecutive nucleotides of a vertebrate gene, preferably a mammalian FasL gene.

The term “transfected stem cell” is meant a stem cell into which exogenous DNA or an exogenous DNA gene has been introduced by retroviral infection or other means well known to those of ordinary skill in the art.

The term “ex vivo gene therapy” is meant the in vitro transfection or retroviral infection of stem cells to form transfected stem cells prior to introducing the transfected stem cells into a mammal.

The term “quiescent stem cell” is meant a stem cell in the G.sub.1 or G.sub.0 phase of the cell cycle. A population of cells is considered herein to be a population of quiescent cells when at least 50%, preferably at least 70%, more preferably at least 80% of the cells are in the G.sub.1 or G.sub.0 phase of the cell cycle. Quiescent cells exhibit a single DNA peak by flow-cytometry analysis, a standard technique well known to those of ordinary skill in the arts of immunology and cell biology. Another technique useful for determining whether a population of cells is quiescent is the addition of a chemical agent to the cell culture medium that is toxic only to actively cycling cells, i.e., DNA synthesizing cells, and does not kill quiescent cells. Non-exclusive examples of such chemical agents include hydroxyurea and high specific activity tritiated thymidine (.sup.3 HtdR). A population of cells is evaluated as to the percent in an actively cycling state by the percent of the cell population killed by the chemical agent. A cell population in which in vitro tritiated thymidine killing is less than approximately 30%, preferably less than approximately 10%, more preferably less than approximately 5%, is considered to be quiescent.

The term “early repopulating stem cells” is meant stem cells which are capable of engrafting into the bone marrow of a host mammal within approximately 6 weeks post-transplantation.

The term “late repopulating stem cells,” also termed “long-term repopulating cells” is meant myelolymphoid stem cells which are capable of engrafting into the bone marrow of a host mammal after approximately 6 weeks post-transplantation.

By the term “engrafting” or engraftment” is meant the persistence of proliferating stem cells in a particular location over time. Thus, early repopulating stem cells do not persist for more than about 6 weeks, whereas late repopulating stem cells persist for longer, and preferably much longer, than about 6 weeks.

Cycling stem cells can be treated to become quiescent by serum or isoleucine starvation. Quiescence can also be induced by reduction of nutrients in the culture medium such that the cycling stem cells enter and remain in the G.sub.1 or G.sub.0 phase of the cell cycle while the nutrient level is reduced. These methods can be used alone or in combination.

By the term “expanded population” is meant a population of cells, wherein at least 50% of the cells have divided at least once. In certain embodiments of the invention, the cells may be induced to divide by the administration of cell cycling agents such as 5-FU and/or cytokines such as IL-3-CHO, IL-6, IL-11, and other growth stimulating factors well known to those of ordinary skill in the art of immunology.

By the term “non-myeloablated host mammal” is meant a mammal which has not undergone irradiation, or other treatment (such as chemical treatment) well known to those of ordinary skill in the art, to cause the death of the bone marrow cells of the mammal.

By the term “myeloablated host mammal” is meant a mammal which has undergone irradiation, or other treatment, such as chemical treatment, well known to those of ordinary skill in the art, to cause the death of at least 50% of the bone marrow cells of the mammal.

“Transcriptional regulatory sequence” is a generic term used throughout the specification to refer to DNA sequences, such as initiation signals, enhancers, and promoters, which induce or control transcription of protein coding sequences with which they are operably linked. In preferred embodiments, transcription of one of the FasL genes is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring forms of FasL polypeptide.

As used herein, the term “transfection” means the introduction of a nucleic acid, e.g., via an expression vector, into a recipient cell by nucleic acid-mediated gene transfer. “Transformation”, as used herein, refers to a process in which a cell's genotype is changed as a result of the cellular uptake of exogenous DNA or RNA, and, for example, the transformed cell expresses a recombinant form of a FasL polypeptide or, in the case of anti-sense expression from the transferred gene, the expression of a naturally-occurring form of the FasL polypeptide is disrupted.

As used herein, the term “transgene” means a nucleic acid sequence (encoding, e.g., one of the FasL polypeptides, or an antisense transcript thereto) which has been introduced into a cell. A transgene could be partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can also be present in a cell in the form of an episome. A transgene can include one or more transcriptional regulatory sequences and any other nucleic acid, such as introns, that may be necessary for optimal expression of a selected nucleic acid.

A “transgenic animal” refers to any animal, preferably a non-human mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of one of the FasL polypeptides, e.g. either agonistic or antagonistic forms. However, transgenic animals in which the recombinant FasL gene is silent are also contemplated, as for example, the FLP or CRE recombinase dependent constructs described below. Moreover, “transgenic animal” also includes those recombinant animals in which gene disruption of one or more FasL genes is caused by human intervention, including both recombination and antisense techniques.

The term “treating” as used herein is intended to encompass curing as well as ameliorating at least one symptom of the condition or disease.

The term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The term “wild-type allele” refers to an allele of a gene which, when present in two copies in a subject results in a wild-type phenotype. There can be several different wild-type alleles of a specific gene, since certain nucleotide changes in a gene may not affect the phenotype of a subject having two copies of the gene with the nucleotide changes.

4.3. Nucleic Acids of the Present Invention

The invention provides FasL and other nucleic acids, homologs thereof, and portions thereof. Preferred nucleic acids have a sequence at least about 60%, 61%, 62%, 63%, 64%, 65%, 66%, 67%, 68%, 69%, 70%, 71%, 72%, 73%, 74%, 75%, 76%, 77%, 78%, 79%, 80%, and more preferably 85% homologous and more preferably 90% and more preferably 95% and even more preferably at least 99% homologous with a nucleotide sequence of a subject gene, e.g., a FasL gene such as a sequence shown in one of FIG. 8A or 9A or complement thereof of the FasL nucleic acids having the GenBank Accession Nos.: GenBank U11821, a FasL gene; or GenBank AF288573, a naturally occurring non-cleaved human Fas ligand expressed only in membrane bound form. Nucleic acids at least 90%, more preferably 95%, and most preferably at least about 98-99% identical with a nucleic sequence represented in one of the subject SEQ ID Nos. or complement thereof are of course also within the scope of the invention. In preferred embodiments, the nucleic acid is mammalian and in particularly preferred embodiments, includes all or a portion of the nucleotide sequence corresponding to the coding region which correspond to the FasL ORF sequences contained within the FasL cDNA sequence shown in FIG. 8A.

The invention also pertains to isolated nucleic acids comprising a nucleotide sequence encoding FasL polypeptides, variants and/or equivalents of such nucleic acids. The term equivalent is understood to include nucleotide sequences encoding functionally equivalent FasL polypeptides or functionally equivalent peptides having an activity of an FasL protein such as described herein. Equivalent nucleotide sequences will include sequences that differ by one or more nucleotide substitution, addition or deletion, such as allelic variants; and will, therefore, include sequences that differ from the nucleotide sequence of the FasL gene shown FIGS. 8A and 9A due to the degeneracy of the genetic code.

Preferred nucleic acids are vertebrate FasL nucleic acids. Particularly preferred vertebrate FasL nucleic acids are mammalian. Regardless of species, particularly preferred FasL nucleic acids encode polypeptides that are at least 60%, 65%, 70%, 72%, 74%, 76%, 78%, 80%, 90%, or 95% similar or identical to an amino acid sequence of a vertebrate FasL protein. In one embodiment, the nucleic acid is a cDNA encoding a polypeptide having at least one bio-activity of the subject FasL polypeptide. Preferably, the nucleic acid includes all or a portion of the nucleotide sequence corresponding to the nucleic acid of FIG. 8A or 9A.

Still other preferred nucleic acids of the present invention encode a FasL polypeptide which is comprised of at least 2, 5, 10, 25, 50, 100, 150 or 200 amino acid residues. For example, such nucleic acids can comprise about 50, 60, 70, 80, 90, or 100 base pairs. Also within the scope of the invention are nucleic acid molecules for use as probes/primer or antisense molecules (i.e. noncoding nucleic acid molecules), which can comprise at least about 6, 12, 20, 30, 50, 60, 70, 80, 90 or 100 base pairs in length.

Another aspect of the invention provides a nucleic acid which hybridizes under stringent conditions to a nucleic acid represented by any of the subject SEQ ID Nos. of the invention or sequences represented in FIG. 8A or 9A or complement thereof or a nucleic acids having a particular ATCC Designation No. Appropriate stringency conditions which promote DNA hybridization, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6 or in Molecular Cloning: A Laboratory Manual, Cold Spring Harbor Press (1989). For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature and salt concentration may be held constant while the other variable is changed. In a preferred embodiment, an FasL nucleic acid of the present invention will bind to one of the subject SEQ ID Nos. or complement thereof under moderately stringent conditions, for example at about 2.0×SSC and about 40° C. In a particularly preferred embodiment, a FasL nucleic acid of the present invention will bind to one of the nucleic acid sequences of FIG. 8A or 9A or complement thereof under high stringency conditions. In another particularly preferred embodiment, a FasL nucleic acid sequence of the present invention will bind to one of the SEQ ID Nos. of the invention which correspond to a FasL ORF nucleic acid sequences, under high stringency conditions.

Nucleic acids having a sequence that differs from the nucleotide sequences shown in one of SEQ ID Nos. of the invention or complement thereof due to degeneracy in the genetic code are also within the scope of the invention. Such nucleic acids encode functionally equivalent peptides (i.e., peptides having a biological activity of a FasL polypeptide) but differ in sequence from the sequence shown in the sequence listing due to degeneracy in the genetic code. For example, a number of amino acids are designated by more than one triplet. Codons that specify the same amino acid, or synonyms (for example, CAU and CAC each encode histidine) may result in “silent” mutations which do not affect the amino acid sequence of an FasL polypeptide. However, it is expected that DNA sequence polymorphisms that do lead to changes in the amino acid sequences of the subject FasL polypeptides will exist among mammals. One skilled in the art will appreciate that these variations in one or more nucleotides (e.g., up to about 3-5% of the nucleotides) of the nucleic acids encoding polypeptides having an activity of a FasL polypeptide may exist among individuals of a given species due to natural allelic variation.

The invention further provides nucleic acids encoding components of the Fas-signaling cascade. The FasL/Fas pathway functions physiologically to limit the size of the specific CTL pool. Fas (receptor), a transmembrane homotrimer, is expressed as on many, but not all cell types. FasL, the cognate transmembrane monomeric ligand, is expressed on few cell types. FasL can be cleaved from cells by a ubiquitous metalloprotease(s), generating a less potent but agonistic soluble FasL (sFasL) fragment. One molecule of FasL binds to each subunit of the Fas trimer. The Fas homotrimer can become part of a 9-meric death inducing signaling complex (DISC) consisting of 3 monomers each of Fas, Fas-associated death domain (FADD), and pro-caspase 8. Formation of the complete 12-meric DISC plus FasL assembly can trigger caspase-mediated apoptosis. Modulators of this pathway include the signal-amplifying mitochondrial pathway, and other elements continue to be discovered. For example, physiologic cellular expression of the catalytically inactive caspase 8 mimic, FLICE inhibitory protein (FLIP), can block Fas-mediated apoptosis. In addition, the activation of caspases is counteracted by pro-survival members of the Bcl-2 family (especially Bcl-x_{L}) and members of the inhibitors of apoptosis (IAP) family. Accordingly the invention provides nucleic acids encoding these components of the Fas-signaling pathway. Numerous such clones are known in the art or can be readily obtained from the sequence information available through GenBank and other sources.

4.3.1 Probes and Primers

The nucleotide sequences determined from the cloning of FasL genes from mammalian organisms will further allow for the generation of probes and primers designed for use in identifying and/or cloning other FasL homologs in other cell types, e.g., from other tissues, as well as FasL homologs from other mammalian organisms. For instance, the present invention also provides a probe/primer comprising a substantially purified oligonucleotide, which oligonucleotide comprises a region of nucleotide sequence that hybridizes under stringent conditions to at least approximately 12, preferably 25, more preferably 40, 50 or 75 consecutive nucleotides of sense or anti-sense sequence selected from SEQ ID No. of the invention. For instance, primers based on the nucleic acid represented in SEQ ID Nos. 1 or 3 can be used in PCR reactions to clone FasL polypeptide encoding genes.

In preferred embodiments, the FasL primers are designed so as to optimize specificity and avoid secondary structures which affect the efficiency of priming. Optimized PCR primers of the present invention are designed so that “upstream” and “downstream” primers have approximately equal melting temperatures such as can be estimated using the formulae: Tₘ=81.5° C.−16.6(log₁₀[Na⁺])+0.41(% G+C)−0.63(% formamide)−(600/length); or Tₘ(° C.)=2(A/T)+4(G/C). Optimized FasL primers may also be designed by using various programs, such as “Primer3” provided by the Whitehead Institute for Biomedical Research at http://www-genome.wi.mit.edu/cgi-bin/primer/primer3.cgi.

In preferred embodiments, the FasL probes and primers can be used to detect FasL locus polymorphisms which occur within and surrounding the FasL gene sequence. Genetic variations within the FasL locus may be associated with the likelihood of the development of a number of human diseases and conditions, such as inflammatory and autoimmune diseases in which FasL encoded polypeptides play an important etiological role. Accordingly the invention provides probes and primers for FasL locus polymorphisms, including polymorphisms associated with the human and mouse FasL gene. PCR primers of the invention include those which flank an FasL human polymorphism and allow amplification and analysis of this region of the genome. Analysis of polymorphic allele identity may be conducted, for example, by direct sequencing or by the use of allele-specific capture probes or by the use of molecular beacon probes. Alternatively, the polymorphic allele may allow for direct detection by the creation or elimination of a restriction endonuclease recognition site(s) within the PCR product or after an appropriate sequence modification is designed into at least one of the primers such that the altered sequence of the primer, when incorporated into the PCR product resulting from amplification of a specific FasL polymorphic allele, creates a unique restriction site in combination with at least one allele but not with at least one other allele of that polymorphism. FasL polymorphisms corresponding to variable number of tandem repeat (VNTR) polymorphisms may be detected by the electrophoretic mobility and hence size of a PCR product obtained using primers which flank the VNTR. Still other FasL polymorphisms corresponding to restriction fragment length polymorphisms (RFLPs) may be detected directly by the mobility of bands on a Southern blot using appropriate FasL locus probes and genomic DNA or cDNA obtained from an appropriate sample organism such as a human or a non-human animal.

Likewise, probes based on the subject FasL sequences can be used to detect transcripts or genomic sequences encoding the same or homologous proteins, for use, e.g, in prognostic or diagnostic assays (further described below). The invention provides probes which are common to alternatively spliced variants of the FasL transcript, such as those corresponding to at least 12 consecutive nucleotides complementary to a sequence found in any of SEQ ID Nos. of the invention. In addition, the invention provides probes which hybridize specifically to alternatively spliced forms of the FasL transcript. Probes and primers can be prepared and modified, e.g., as previously described herein for other types of nucleic acids.

4.3.2 Antisense, Ribozyme and Triplex Techniques

Another aspect of the invention relates to the use of the isolated nucleic acid in “antisense” therapy. As used herein, “antisense” therapy refers to administration or in situ generation of oligonucleotide molecules or their derivatives which specifically hybridize (e.g., bind) under cellular conditions, with the cellular mRNA and/or genomic DNA encoding one or more of the subject FasL proteins so as to inhibit expression of that protein, e.g., by inhibiting transcription and/or translation. The binding may be by conventional base pair complementarity, or, for example, in the case of binding to DNA duplexes, through specific interactions in the major groove of the double helix. In general, “antisense” therapy refers to the range of techniques generally employed in the art, and includes any therapy which relies on specific binding to oligonucleotide sequences.

An antisense construct of the present invention can be delivered, for example, as an expression plasmid which, when transcribed in the cell, produces RNA which is complementary to at least a unique portion of the cellular mRNA which encodes an FasL protein. Alternatively, the antisense construct is an oligonucleotide probe which is generated ex vivo and which, when introduced into the cell causes inhibition of expression by hybridizing with the mRNA and/or genomic sequences of an FasL gene. Such oligonucleotide probes are preferably modified oligonucleotides which are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) BioTechniques 6:958-976; and Stein et al. (1988) Cancer Res 48:2659-2668. With respect to antisense DNA, oligodeoxyribonucleotides derived from the translation initiation site, e.g., between the −10 and +10 regions of the FASL nucleotide sequence of interest, are preferred.

Antisense approaches involve the design of oligonucleotides (either DNA or RNA) that are complementary to FasL mRNA. The antisense oligonucleotides will bind to the FasL mRNA transcripts and prevent translation. Absolute complementarity, although preferred, is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well. (Wagner, R. 1994. Nature 372:333). Therefore, oligonucleotides complementary to either the 5′ or 3′ untranslated, non-coding regions of an FasL gene could be used in an antisense approach to inhibit translation of endogenous FasL mRNA. Oligonucleotides complementary to the 5′ untranslated region of the mRNA should include the complement of the AUG start codon. Antisense oligonucleotides complementary to mRNA coding regions are less efficient inhibitors of translation but could also be used in accordance with the invention. Whether designed to hybridize to the 5′, 3′ or coding region of FasL mRNA, antisense nucleic acids should be at least six nucleotides in length, and are preferably less than about 100 and more preferably less than about 50, 25, 17 or 10 nucleotides in length.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810, published Dec. 15, 1988) or the blood-brain barrier (see, e.g., PCT Publication No. WO89/10134, published Apr. 25, 1988), hybridization-triggered cleavage agents. (See, e.g., Krol et al., 1988, BioTechniques 6:958-976) or intercalating agents. (See, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytiethyl) uracil, 5-carboxymethylaminomethyl-2-th iouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their ability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15:6625-6641). The oligonucleotide is a 2′-O-methylribonucleotide (Inoue et al., 1987, Nucl. Acids Res. 15:6131-6148), or a chimeric RNA-DNA analogue (Inoue et al., 1987, FEBS Lett. 215:327-330).

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate olgonucleotides can be prepared by use of controlled pore glass polymer supports (Sarin et al., 1988, Proc. Natl. Acad. Sci. U.S.A. 85:7448-7451), etc.

While antisense nucleotides complementary to the FasL coding region sequence can be used, those complementary to the transcribed untranslated region and to the region comprising the initiating methionine are most preferred.

The antisense molecules can be delivered to cells which express FasL in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically.

However, it may be difficult to achieve intracellular concentrations of the antisense sufficient to suppress translation on endogenous mRNAs in certain instances. Therefore a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous FasL transcripts and thereby prevent translation of the FasL mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive and can include but not be limited to: the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al, 1982, Nature 296:39-42), etc. Any type of plasmid, cosmid, YAC or viral vector can be used to prepare the recombinant DNA construct which can be introduced directly into the tissue site. Alternatively, viral vectors can be used which selectively infect the desired tissue, in which case administration may be accomplished by another route (e.g., systematically).

Ribozyme molecules designed to catalytically cleave FasL mRNA transcripts can also be used to prevent translation of FasL mRNA and expression of FasL (See, e.g., PCT International Publication WO90/11364, published Oct. 4, 1990; Sarver et al., 1990, Science 247:1222-1225 and U.S. Pat. No. 5,093,246). While ribozymes that cleave mRNA at site specific recognition sequences can be used to destroy FasL mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591. For example, there are a number of potential hammerhead ribozyme cleavage sites within the nucleotide sequence of human FasL-1 and FasL-3. Preferably the ribozyme is engineered so that the cleavage recognition site is located near the 5′ end of the FasL mRNA; i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent application No. WO88/04300 by University Patents Inc.; Been and Cech, 1986, Cell, 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in an FasL gene.

As in the antisense approach, the ribozymes can be composed of modified oligonucleotides (e.g., for improved stability, targeting, etc.) and should be delivered to cells which express the FasL gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous FasL messages and inhibit translation. Because ribozymes unlike antisense molecules, are catalytic, a lower intracellular concentration is required for efficiency.

Endogenous FasL gene expression can also be reduced by inactivating or “knocking out” the FasL gene or its promoter using targeted homologous recombination. (E.g., see Smithies et al., 1985, Nature 317:230-234; Thomas & Capecchi, 1987, Cell 51:503-512; Thompson et al., 1989 Cell 5:313-321; each of which is incorporated by reference herein in its entirety). For example, a mutant, non-functional FasL (or a completely unrelated DNA sequence) flanked by DNA homologous to the endogenous FasL gene (either the coding regions or regulatory regions of the FasL gene) can be used, with or without a selectable marker and/or a negative selectable marker, to transfect cells that express FasL in vivo. Insertion of the DNA construct, via targeted homologous recombination, results in inactivation of the FasL gene. Such approaches are particularly suited in the agricultural field where modifications to ES (embryonic stem) cells can be used to generate animal offspring with an inactive FasL (e.g., see Thomas & Capecchi 1987 and Thompson 1989, supra). However this approach can be adapted for use in humans provided the recombinant DNA constructs are directly administered or targeted to the required site in vivo using appropriate viral vectors.

Alternatively, endogenous FasL gene expression can be reduced by targeting deoxyribonucleotide sequences complementary to the regulatory region of the FasL gene (i.e., the FasL promoter and/or enhancers) to form triple helical structures that prevent transcription of the FasL gene in target cells in the body. (See generally, Helene, C. 1991, Anticancer Drug Des., 6(6):569-84; Helene, C., et al., 1992, Ann. N.Y. Acad. Sci., 660:27-36; and Maher, L. J., 1992, Bioassays 14(12):807-15).

Nucleic acid molecules to be used in triple helix formation for the inhibition of transcription are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizable stretch of either purines or pyrimidines to be present on one strand of a duplex.

Antisense RNA and DNA, ribozyme, and triple helix molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

4.3.3. Vectors Encoding FasL Proteins and Methods of Producing FasL Expressing Cells

The invention further provides plasmids and vectors encoding an FasL protein, which can be used to express an FasL protein in a host cell. The host cell may be any prokaryotic or eukaryotic cell. Thus, a nucleotide sequence derived from the cloning of mammalian FasL proteins, encoding all or a selected portion of the full-length protein, can be used to produce a recombinant form of an FasL polypeptide via microbial or eukaryotic cellular processes. Ligating the polynucleotide sequence into a gene construct, such as an expression vector, and transforming or transfecting into hosts, either eukaryotic (yeast, avian, insect or mammalian) or prokaryotic (bacterial) cells, are standard procedures well known in the art.

Typically, expression vectors used for expressing, in vivo or in vitro a FasL protein contain a nucleic acid encoding a FasL polypeptide, operably linked to at least one transcriptional regulatory sequence. Regulatory sequences are art-recognized and are selected to direct expression of the subject proteins in the desired fashion (time and place). Transcriptional regulatory sequences are described in Goeddel; Gene Expression Technology: Methods in Enzymology 185, Academic Press, San Diego, Calif. (1990).

Suitable vectors for the expression of a FasL polypeptide include plasmids of the types: pBR322-derived plasmids, pEMBL-derived plasmids, pEX-derived plasmids, pBTac-derived plasmids and pUC-derived plasmids for expression in prokaryotic cells, such as E. coli.

The preferred mammalian expression vectors contain both prokaryotic sequences, to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papillomavirus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2^{nd} Ed., ed. by Sambrook, Fritsch and Maniatis (Cold Spring Harbor Laboratory Press: 1989) Chapters 16 and 17.

In a preferred embodiment, the promoter is a constitutive promoter, e.g., a strong viral promoter, e.g., CMV promoter. The promoter can also be cell- or tissue-specific, that permits substantial transcription of the DNA only in predetermined cells, e.g., in professional antigen presenting cells, such as a promoter specific for fibroblasts, or smooth muscle cells, retinal cells or RPE cells. A smooth muscle specific promoter is. e.g., the promoter of the smooth muscle cell marker SM22alpha (Akyura et al. (2000) Mol Med 6:983. Retinal pigment epithelial cell specific promoter is, e.g., the promoter of the Rpe65 gene (Boulanger et al. (2000) J Biol Chem 275:31274). The promoter can also be an inducible promoter, e.g., a metallothionein promoter. Other inducible promoters include those that are controlled by the inducible binding, or activation, of a transcription factor, e.g., as described in U.S. Pat. Nos. 5,869,337 and 5,830,462 by Crabtree et al., describing small molecule inducible gene expression (a genetic switch); International patent applications PCT/US94/01617, PCT/US95/10591, PCT/US96/09948 and the like, as well as in other heterologous transcription systems such as those involving tetracyclin-based regulation reported by Bujard et al., generally referred to as an allosteric “off-switch” described by Gossen and Bujard (Proc. Natl. Acad. Sci. U.S.A. (1992) 89:5547) and in U.S. Pat. Nos. 5,464,758; 5,650,298; and 5,589,362 by Bujard et al. Other inducible transcription systems involve steroid or other hormone-based regulation.

The polynucleotide of the invention together with all necessary transcriptional and translational control sequences is referred to herein as “construct of the invention” or “transgene of the invention.”

The polynucleotide of the invention may also be introduced into the cell in which it is to be expressed together with another DNA sequence (which may be on the same or a different DNA molecule as the polynucleotide of the invention) coding for another agent. Exemplary agents are further described below. In one embodiment, the DNA encodes a polymerase for transcribing the DNA, and may comprise recognition sites for the polymerase and the injectable preparation may include an initial quantity of the polymerase.

In certain instances, it may be preferred that the polynucleotide is translated for a limited period of time so that the polypeptide delivery is transitory. This can be achieved, e.g., by the use of an inducible promoter.

The polynucleotides used in the present invention may also be produced in part or in total by chemical synthesis, e.g., by the phosphoramidite method described by Beaucage and Carruthers, Tetra. Letts., 22:1859-1862 (1981) or the triester method according to the method described by Matteucci et al., J. Am. Chem. Soc., 103:3185 (1981), and may be performed on commercial automated oligonucleotide synthesizers. A double-stranded fragment may be obtained from the single stranded product of chemical synthesis either by synthesizing the complementary strand and annealing the strand together under appropriate conditions or by adding the complementary strand using DNA polymerase with an appropriate primer sequence.

The polynucleotide of the invention operably linked to all necessary transcriptional and translational regulation elements can be injected as naked DNA into a subject. In a preferred embodiment, the polynucleotide of the invention and necessary regulatory elements are present in a plasmid or vector. Thus, the polynucleotide of the invention may be DNA, which is itself non-replicating, but is inserted into a plasmid, which may further comprise a replicator. The DNA may be a sequence engineered so as not to integrate into the host cell genome.

Preferred vectors for use according to the invention are expression vectors, i.e., vectors that allow expression of a nucleic acid in a cell vectors. Preferred expression vectors are those which contain both prokaryotic sequences, to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and phyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papillomavirus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2 Ed., ed. by Sambrook, Fritsch and Maniatis (Cold Spring Harbor Laboratory Press: 1989) Chapters 16 and 17.

Any means for the introduction of polynucleotides into mammals, human or non-human, may be adapted to the practice of this invention for the delivery of the various constructs of the invention into the intended recipient. In one embodiment of the invention, the DNA constructs are delivered to cells by transfection, i.e., by delivery of “naked” DNA or in a complex with a colloidal dispersion system. A colloidal system includes macromolecule complexes, nanocapsules, microspheres, beads, and lipid-based systems including oil-in-water emulsions, micelles, mixed micelles, and liposomes. The preferred colloidal system of this invention is a lipid-complexed or liposome-formulated DNA. In the former approach, prior to formulation of DNA, e.g., with lipid, a plasmid containing a transgene bearing the desired DNA constructs may first be experimentally optimized for expression (e.g., inclusion of an intron in the 5′ untranslated region and elimination of unnecessary sequences (Felgner, et al., Ann NY Acad Sci 126-1139, 1995). Formulation of DNA, e.g. with various lipid or liposome materials, may then be effected using known methods and materials and delivered to the recipient mammal. See, e.g., Canonico et al, Am J Respir Cell Mol Biol 10:24-29, 1994; Tsan et al, Am J Physiol 268; Alton et al., Nat Genet. 5:135-142, 1993 and U.S. Pat. No. 5,679,647 by Carson et al. Colloidal dispersion systems.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs, which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Naked DNA or DNA associated with a delivery vehicle, e.g., liposomes, can be administered to several sites in a subject (see below). For example, smooth muscle cells can be targeted with an antibody binding specifically to SM22a, a smooth muscle cell marker. Retinal cells and RPE cells can similarly be targeted.

In a preferred method of the invention, the DNA constructs are delivered using viral vectors. The transgene may be incorporated into any of a variety of viral vectors useful in gene therapy, such as recombinant retroviruses, adenovirus, adeno-associated virus (AAV), and herpes simplex virus-1, or recombinant bacterial or eukaryotic plasmids. While various viral vectors may be used in the practice of this invention, AAV- and adenovirus-based approaches are of particular interest. Such vectors are generally understood to be the recombinant gene delivery system of choice for the transfer of exogenous genes in vivo, particularly into humans. The following additional guidance on the choice and use of viral vectors may be helpful to the practitioner. As described in greater detail below, such embodiments of the subject expression constructs are specifically contemplated for use in various in vivo and ex vivo gene therapy protocols.

A. Adenoviral Vectors

A viral gene delivery system useful in the present invention utilizes adenovirus-derived vectors. Knowledge of the genetic organization of adenovirus, a 36 kB, linear and double-stranded DNA virus, allows substitution of a large piece of adenoviral DNA with foreign sequences up to 8 kB. In contrast to retrovirus, the infection of adenoviral DNA into host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in the human.

Adenoviruses have been shown in particular to be efficient in gene delivery to the RPE cells. For example, Baffi et al. describe the delivery of an adenovirus encoding vascular endothelial growth factor to the subretinal space in the rat, resulting in the expression of VEGF in the RPE cells of the rat (Baffi et al. (2000) Invest Ophthalmol Vis Sci 41:3582). Another reference describes that laser photocoagulation further increases the susceptibility of proliferating RPE cells to adenovirus-mediated gene delivery (Lai et al. (1999) Curr Eye Res 19:411). Sakamoto et al. describe that a vitrectomy also improves adenovirus-mediated gene delivery to the retina (Sakamoto et al. (1998) Gene Ther. 5: 1088). Ali et al. report that co-injection of adenovirus expressing CTLA4-Ig prolongs adenovirally mediated gene expression in the mouse retina, by blocking T cell activation (Ali et al. (1998) Gene Ther. 5:1561). Other references decribing expression of a transgene in retinal cells and RPE cells, upon injection of an adenoviral vector comprising the transgene in the vitreous cavity of eyes of non-human animals include Lai et al. (2000): Invest Ophthalmol Vis Sci 41 :580; Yu et al. (2000) Growth Factors 17:301; and Rackoczy et al. (1998) Aust N Z J Ophthalmol 26 Suppl 1:S56.

Adenovirus is particularly suitable for use as a gene transfer vector because of its mid-sized genome, ease of manipulation, high titer, wide target-cell range, and high infectivity. Both ends of the viral genome contain 100-200 base pair (bp) inverted terminal repeats (ITR), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut off (Renan (1990) Radiotherap. Oncol. 19:197). The products of the late genes, including the majority of the viral capsid proteins, are expressed only after significant processing of a single primary transcript issued by the major late promoter (MLP). The MLP (located at 16.8 m.u.) is particularly efficient during the late phase of infection, and all the mRNAs issued from this promoter possess a 5′ tripartite leader (TL) sequence which makes them preferred mRNAs for translation.

The genome of an adenovirus can be manipulated such that it encodes a gene product of interest, but is inactivated in terms of its ability to replicate in a normal lytic viral life cycle (see, for example, Berkner et al., (1988) BioTechniques 6:616; Rosenfeld et al., (1991) Science 252:431-434; and Rosenfeld et al., (1992) Cell 68:143-155). Suitable adenoviral vectors derived from the adenovirus strain Ad type 5 dl324 or other strains of adenovirus (e.g., Ad2, Ad3, Ad7 etc.) are well known to those skilled in the art. Recombinant adenoviruses can be advantageous in certain circumstances in that they are not capable of infecting nondividing cells and can be used to infect a wide variety of cell types, including airway epithelium (Rosenfeld et al., (1992) cited supra), endothelial cells (Lemarchand et al., (1992) PNAS USA 89:6482-6486), hepatocytes (Herz and Gerard, (1993) PNAS USA 90:2812-2816) and muscle cells (Quantin et al., (1992) PNAS USA 89:2581-2584).

Adenovirus vectors have also been used in vaccine development (Grunhaus and Horwitz (1992) Siminar in Virology 3:237; Graham and Prevec (1992) Biotechnology 20:363). Experiments in administering recombinant adenovirus to different tissues include trachea instillation (Rosenfeld et al. (1991); Rosenfeld et al. (1992) Cell 68:143), muscle injection (Ragot et al. (1993) Nature 361:647), peripheral intravenous injection (Herz and Gerard (1993) Proc. Natl. Acad. Sci. U.S.A. 90:2812), and stereotactic inoculation into the brain (Le Gal La Salle et al. (1993) Science 254:988).

Furthermore, the virus particle is relatively stable and amenable to purification and concentration, and as above, can be modified so as to affect the spectrum of infectivity. Additionally, adenovirus is easy to grow and manipulate and exhibits broad host range in vitro and in vivo. This group of viruses can be obtained in high titers, e.g., 10⁹-10¹¹ plaque-forming unit (PFU)/ml, and they are highly infective. The life cycle of adenovirus does not require integration into the host cell genome. The foreign genes delivered by adenovirus vectors are episomal, and therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors. Moreover, the carrying capacity of the adenoviral genome for foreign DNA is large (up to 8 kilobases) relative to other gene delivery vectors (Berkner et al., supra; Haj-Ahmand and Graham (1986) J. Virol. 57:267). Most replication-defective adenoviral vectors currently in use and therefore favored by the present invention are deleted for all or parts of the viral E1 and E3 genes but retain as much as 80% of the adenoviral genetic material (see, e.g., Jones et al., (1979) Cell 16:683; Berkner et al., supra; and Graham et al., in Methods in Molecular Biology, E. J. Murray, Ed. (Humana, Clifton, N.J., 1991) vol. 7. pp. 109-127). Expression of the inserted polynucleotide of the invention can be under control of, for example, the E1A promoter, the major late promoter (MLP) and associated leader sequences, the viral E3 promoter, or exogenously added promoter sequences.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the method of the present invention. This is because Adenovirus type 5 is a human adenovirus about which a great deal of biochemical and genetic information is known, and it has historically been used for most constructions employing adenovirus as a vector. As stated above, the typical vector according to the present invention is replication defective and will not have an adenovirus E1 region. Thus, it will be most convenient to introduce the nucleic acid of interest at the position from which the E1 coding sequences have been removed. However, the position of insertion of the polynucleotide or construct on the invention (also referred to as “nucleic acid of interest”) in a region within the adenovirus sequences is not critical to the present invention. For example, it may also be inserted in lieu of the deleted E3 region in E3 replacement vectors as described previously by Karlsson et. al. (1986) or in the E4 region where a helper cell line or helper virus complements the E4 defect.

A preferred helper cell line is 293 (ATCC Accession No. CRL1573). This helper cell line, also termed a “packaging cell line” was developed by Frank Graham (Graham et al. (1987) J. Gen. Virol. 36:59-72 and Graham (1977) J. General Virology 68:937-940) and provides E1A and E1B in trans. However, helper cell lines may also be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells.

Adenoviruses can also be cell type specific, i.e., infect only restricted types of cells and/or express a transgene only in restricted types of cells. For example, the viruses comprise a gene under the transcriptional control of a transcription initiation region specifically regulated by target host cells, as described e.g., in U.S. Pat. No. 5,698,443, by Henderson and Schuur, issued Dec. 16, 1997. Thus, replication competent adenoviruses can be restricted to certain cells by, e.g., inserting a cell specific response element to regulate a synthesis of a protein necessary for replication, e.g., E1A or E1B.

DNA sequences of a number of adenovirus types are available from Genbank. For example, human adenovirus type 5 has GenBank Accession No.M73260. The adenovirus DNA sequences may be obtained from any of the 42 human adenovirus types currently identified. Various adenovirus strains are available from the American Type Culture Collection, Rockville, Md., or by request from a number of commercial and academic sources. A transgene as described herein may be incorporated into any adenoviral vector and delivery protocol, by restriction digest, linker ligation or filling in of ends, and ligation.

Adenovirus producer cell lines can include one or more of the adenoviral genes E1, E2a, and E4 DNA sequence, for packaging adenovirus vectors in which one or more of these genes have been mutated or deleted are described, e.g., in PCT/US95/15947 (WO 96/18418) by Kadan et al.; PCT/US95/07341 (WO 95/346671) by Kovesdi et al.; PCT/FR94/00624 (WO94/28152) by Imler et al.; PCT/FR94/00851 (WO 95/02697) by Perrocaudet et al., PCT/US95/14793 (WO96/14061) by Wang et al.

B. AAV Vectors

Yet another viral vector system useful for delivery of the subject polynucleotides is the adeno-associated virus (AAV). Adeno-associated virus is a naturally occurring defective virus that requires another virus, such as an adenovirus or a herpes virus, as a helper virus for efficient replication and a productive life cycle. (For a review, see Muzyczka et al., Curr. Topics in Micro. and Immunol. (1992) 158:97-129).

AAV has not been associated with the cause of any disease. AAV is not a transforming or oncogenic virus. AAV integration into chromosomes of human cell lines does not cause any significant alteration in the growth properties or morphological characteristics of the cells. These properties of AAV also recommend it as a potentially useful human gene therapy vector.

AAV is also one of the few viruses that may integrate its DNA into non-dividing cells, e.g., pulmonary epithelial cells, and exhibits a high frequency of stable integration (see for example Flotte et al., (1992) Am. J. Respir. Cell. Mol. Biol. 7:349-356; Samulski et al., (1989) J. Virol. 63:3822-3828; and McLaughlin et al., (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al., (1985) Mol. Cell. Biol. 5:3251-3260 can be used to introduce DNA into cells. A variety of nucleic acids have been introduced into different cell types using AAV vectors (see for example Herinonat et al., (1984) PNAS USA 81:6466-6470; Tratschin et al., (1985) Mol. Cell. Biol. 4:2072-2081; Wondisford et al., (1988) Mol. Endocrinol. 2:32-39; Tratschin et al., (1984) J. Virol. 51:611-619; and Flotte et al., (1993) J. Biol. Chem. 268:3781-3790).

AAV has been used successfully to introduce gene constructs into retinal cells in animals, including non-human primates. For example, an AAV virus containing a gene encoding FGF-2 was administered by subretinal injection into a transgenic rat model for retinitis pigmentosa, which resulted in reduction of the rate of photoreceptor degeneration (Lau et al. (2000) Invest. Ophthalmol. Vis. Csci. 41:3622). AAV has been used for gene transduction in photoreceptor cells in non-human animals (see, e.g., Flannery et al. (1997) PNAS 94:6916; Bennett et al. (2000) PNAS 96:9920). RPE cells have also been transduced efficiently by subretinal injection of an AAV (Bennett et al. (1997) Invest. Ophthalmol. Visual Sci. 38:2857). Grant et al. also describe that a recombinant AAV injected into the vitreous body or the subretinal space of mouse eyes results in the transduction of cells of the retinal pigment epithelium (RPE), ganglion cells and photoreceptor cells for up to three months, i.e., for as long as the experiment was conducted (Grant et al. (1997) Curr. Eye Res. 16, 949). Efficient transduction of RPE cells in non-human animals is also described in Rollins et al. (2000) Clin Experiment Ophthalmol 28:382-6; Ali et al. (1998) Hum Gene Ther 9:81; and Ali et al. (1996) Hum Mol Genet. 5:591.

The AAV-based expression vector to be used typically includes the 145 nucleotide AAV inverted terminal repeats (ITRs) flanking a restriction site that can be used for subcloning of the transgene, either directly using the restriction site available, or by excision of the transgene with restriction enzymes followed by blunting of the ends, ligation of appropriate DNA linkers, restriction digestion, and ligation into the site between the ITRs. The capacity of AAV vectors is about 4.4 kb. The following proteins have been expressed using various AAV-based vectors, and a variety of promoter/enhancers: neomycin phosphotransferase, chloramphenicol acetyl transferase, Fanconi's anemia gene, cystic fibrosis transmembrane conductance regulator, and granulocyte macrophage colony-stimulating factor (Kotin, R. M., Human Gene Therapy 5:793-801, 1994, Table 1). A transgene incorporating the various DNA constructs of this invention can similarly be included in an AAV-based vector. As an alternative to inclusion of a constitutive promoter such as CMV to drive expression of the polynucleotide of interest, an AAV promoter can be used (ITR itself or AAV p5 (Flotte, et al. J. Biol. Chem. 268:3781-3790, 1993)).

Such a vector can be packaged into AAV virions by reported methods. For example, a human cell line such as 293 can be co-transfected with the AAV-based expression vector and another plasmid containing open reading frames encoding AAV rep and cap (which are obligatory for replication and packaging of the recombinant viral construct) under the control of endogenous AAV promoters or a heterologous promoter. In the absence of helper virus, the rep proteins Rep68 and Rep78 prevent accumulation of the replicative form, but upon superinfection with adenovirus or herpes virus, these proteins permit replication from the ITRs (present only in the construct containing the transgene) and expression of the viral capsid proteins. This system results in packaging of the transgene DNA into AAV virions (Carter, B. J., Current Opinion in Biotechnology 3:533-539, 1992; Kotin, R. M, Human Gene Therapy 5:793-801, 1994)). Typically, three days after transfection, recombinant AAV is harvested from the cells along with adenovirus and the contaminating adenovirus is then inactivated by heat treatment.

Methods to improve the titer of AAV can also be used to express the polynucleotide of the invention in an AAV virion. Such strategies include, but are not limited to: stable expression of the ITR-flanked transgene in a cell line followed by transfection with a second plasmid to direct viral packaging; use of a cell line that expresses AAV proteins inducibly, such as temperature-sensitive inducible expression or pharmacologically inducible expression. Alternatively, a cell can be transformed with a first AAV vector including a 5′ ITR, a 3′ ITR flanking a heterologous gene, and a second AAV vector which includes an inducible origin of replication, e.g., SV40 origin of replication, which is capable of being induced by an agent, such as the SV40 T antigen and which includes DNA sequences encoding the AAV rep and cap proteins. Upon induction by an agent, the second AAV vector may replicate to a high copy number, and thereby increased numbers of infectious AAV particles may be generated (see, e.g, U.S. Pat. No. 5,693,531 by Chiorini et al., issued Dec. 2, 1997. In yet another method for producing large amounts of recombinant AAV, a chimeric plasmid is used which incorporate the Epstein Barr Nuclear Antigen (EBNA) gene, the latent origin of replication of Epstein Barr virus (oriP) and an AAV genome. These plasmids are maintained as a multicopy extra-chromosomal elements in cells, such as in 293 cells. Upon addition of wild-type helper functions, these cells will produce high amounts of recombinant AAV (U.S. Pat. No. 5,691,176 by Lebkowski et al., issued Nov. 25, 1997). In another system, an AAV packaging plasmid is provided that allows expression of the rep gene, wherein the p5 promoter, which normally controls rep expression, is replaced with a heterologous promoter (U.S. Pat. No. 5,658,776, by Flotte et al., issued Aug. 19, 1997). Additionally, one may increase the efficiency of AAV transduction by treating the cells with an agent that facilitates the conversion of the single stranded form to the double stranded form, as described in Wilson et al., WO96/39530.

AAV stocks can be produced as described in Hermonat and Muzyczka (1984) PNAS 81:6466, modified by using the pAAV/Ad described by Samulski et al. (1989) J. Virol. 63:3822. Concentration and purification of the virus can be achieved by reported methods such as banding in cesium chloride gradients, as was used for the initial report of AAV vector expression in vivo (Flotte, et al. J. Biol. Chem. 268:3781-3790, 1993) or chromatographic purification, as described in O'Riordan et al., WO97/08298.

Methods for in vitro packaging AAV vectors are also available and have the advantage that there is no size limitation of the DNA packaged into the particles (see, U.S. Pat. No. 5,688,676, by Zhou et al., issued Nov. 18, 1997). This procedure involves the preparation of cell free packaging extracts.

For additional detailed guidance on AAV technology which may be useful in the practice of the subject invention, including methods and materials for the incorporation of a transgene, the propagation and purification of the recombinant AAV vector containing the transgene, and its use in transfecting cells and mammals, see e.g. Carter et al, U.S. Pat. No. 4,797,368 (10 Jan. 1989); Muzyczka et al, U.S. Pat. No. 5,139,941 (18 Aug. 1992); Lebkowski et al, U.S. Pat. No. 5,173,414 (22 Dec. 1992); Srivastava, U.S. Pat. No. 5,252,479 (12 Oct. 1993); Lebkowski et al, U.S. Pat. No. 5,354,678 (11 Oct. 1994); Shenk et al, U.S. Pat. No. 5,436,146(25 Jul. 1995); Chatterjee et al, U.S. Pat. No. 5,454,935 (12 Dec. 1995), Carter et al WO 93/24641 (published 9 Dec. 1993), and Natsoulis, U.S. Pat. No. 5,622,856 (Apr. 22, 1997). Further information regarding AAVs and the adenovirus or herpes helper functions required can be found in the following articles: Berns and Bohensky (1987), “Adeno-Associated Viruses: An Update”, Advanced in Virus Research, Academic Press, 33:243-306. The genome of AAV is described in Laughlin et al. (1983) “Cloning of infectious adeno-associated virus genomes in bacterial plasmids”, Gene, 23: 65-73. Expression of AAV is described in Beaton et al. (1989) “Expression from the Adeno-associated virus p5 and p19 promoters is negatively regulated in trans by the rep protein”, J. Virol., 63:4450-4454. Construction of rAAV is described in a number of publications: Tratschin et al. (1984) “Adeno-associated virus vector for high frequency integration, expression and rescue of genes in mammalian cells”, Mol. Cell. Biol., 4:2072-2081; Hermonat and Muzyczka (1984) “Use of adeno-associated virus as a mammalian DNA cloning vector: Transduction of neomycin resistance into mammalian tissue culture cells”, Proc. Natl. Acad. Sci. USA, 81:6466-6470; McLaughlin et al. (1988) “Adeno-associated virus general transduction vectors: Analysis of Proviral Structures”, J. Virol., 62:1963-1973; and Samulski et al. (1989) “Helper-free stocks of recombinant adeno-associated viruses: normal integration does not require viral gene expression”, J. Virol., 63:3822-3828. Cell lines that can be transformed by rAAV are those described in Lebkowski et al. (1988) “Adeno-associated virus: a vector system for efficient introduction and integration of DNA into a variety of mammalian cell types”, Mol. Cell. Biol., 8:3988-3996. “Producer” or “packaging” cell lines used in manufacturing recombinant retroviruses are described in Dougherty et al. (1989) J. Virol., 63:3209-3212; and Markowitz et al. (1988) J. Virol., 62:1120-1124.

C. Hybrid Adenovirus-AAV Vectors

Hybrid Adenovirus-AAV vectors represented by an adenovirus capsid containing a nucleic acid comprising a portion of an adenovirus, and 5′ and 3′ ITR sequences from an AAV which flank a selected transgene under the control of a promoter. See e.g. Wilson et al, International Patent Application Publication No. WO 96/13598. This hybrid vector is characterized by high titer transgene delivery to a host cell and the ability to stably integrate the transgene into the host cell chromosome in the presence of the rep gene. This virus is capable of infecting virtually all cell types (conferred by its adenovirus sequences) and stable long term transgene integration into the host cell genome (conferred by its AAV sequences).

The adenovirus nucleic acid sequences employed in this vector can range from a minimum sequence amount, which requires the use of a helper virus to produce the hybrid virus particle, to only selected deletions of adenovirus genes, which deleted gene products can be supplied in the hybrid viral process by a packaging cell. For example, a hybrid virus can comprise the 5′ and 3′ inverted terminal repeat (ITR) sequences of an adenovirus (which function as origins of replication). The left terminal sequence (5′) sequence of the Ad5 genome that can be used spans bp 1 to about 360 of the conventional adenovirus genome (also referred to as map units 0-1) and includes the 5′ ITR and the packaging/enhancer domain. The 3′ adenovirus sequences of the hybrid virus include the right terminal 3′ ITR sequence which is about 580 nucleotides (about bp 35,353-end of the adenovirus, referred to as about map units 98.4-100).

The AAV sequences useful in the hybrid vector are viral sequences from which the rep and cap polypeptide encoding sequences are deleted and are usually the cis acting 5′ and 3′ ITR sequences. Thus, the AAV ITR sequences are flanked by the selected adenovirus sequences and the AAV ITR sequences themselves flank a selected transgene. The preparation of the hybrid vector is further described in detail in published PCT application entitled “Hybrid Adenovirus-AAV Virus and Method of Use Thereof”, WO 96/13598 by Wilson et al.

For additional detailed guidance on adenovirus and hybrid adenovirus-AAV technology which may be useful in the practice of the subject invention, including methods and materials for the incorporation of a transgene, the propagation and purification of recombinant virus containing the transgene, and its use in transfecting cells and mammals, see also Wilson et al, WO 94/28938, WO 96/13597 and WO 96/26285, and references cited therein.

D. Retroviruses

The retroviruses are a group of single-stranded RNA viruses characterized by an ability to convert their RNA to double-stranded DNA in infected cells by a process of reverse-transcription (Coffin (1990) Retroviridae and their Replication” In Fields, Knipe ed. Virology. New York: Raven Press). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsial proteins, polymerase enzyme, and envelope components, respectively. A sequence found upstream from the gag gene, termed psi, functions as a signal for packaging of the genome into virions. Two long terminal repeat (LTR) sequences are present at the 5′ and 3′ ends of the viral genome. These contain strong promoter and enhancer sequences and are also required for integration in the host cell genome (Coffin (1990), supra).

In order to construct a retroviral vector, a nucleic acid of interest is inserted into the viral genome in the place of certain viral sequences to produce a virus that is replication-defective. In order to produce virions, a packaging cell line containing the gag, pol, and env genes but without the LTR and psi components is constructed (Mann et al. (1983) Cell 33:153). When a recombinant plasmid containing a human cDNA, together with the retroviral LTR and psi sequences is introduced into this cell line (by calcium phosphate precipitation for example), the psi sequence allows the RNA transcript of the recombinant plasmid to be packaged into viral particles, which are then secreted into the culture media (Nicolas and Rubenstein (1988) “Retroviral Vectors”, In: Rodriguez and Denhardt ed. Vectors: A Survey of Molecular Cloning Vectors and their Uses. Stoneham:Butterworth; Temin, (1986) “Retrovirus Vectors for Gene Transfer: Efficient Integration into and Expression of Exogenous DNA in Vertebrate Cell Genome”, In: Kucherlapati ed. Gene Transfer. New York: Plenum Press; Mann et al., 1983, supra). The media containing the recombinant retroviruses is then collected, optionally concentrated, and used for gene transfer. Retroviral vectors are able to infect a broad variety of cell types. Integration and stable expression require the division of host cells (Paskind et al. (1975) Virology 67:242). This aspect is particularly relevant for the treatment of PVR, since these vectors allow selective targeting of cells which proliferate, i.e., selective targeting of the cells in the epiretinal membrane, since these are the only ones proliferating in eyes of PVR subjects.

A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood 76:271). Thus, recombinant retrovirus can be constructed in which part of the retroviral coding sequence (gag, pol, env) has been replaced by nucleic acid encoding a protein of the present invention, e.g., a transcriptional activator, rendering the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al., (eds.) Greene Publishing Associates, (1989), Sections 9.10-9.14 and other standard laboratory manuals. Examples of suitable retroviruses include pLJ, pZIP, pWE and pEM which are well known to those skilled in the art. A preferred retroviral vector is a pSR MSVtkNeo (Muller et al. (1991) Mol. Cell Biol. 11:1785 and pSR MSV(XbaI) (Sawyers et al. (1995) J. Exp. Med. 181:307) and derivatives thereof. For example, the unique BamHI sites in both of these vectors can be removed by digesting the vectors with BamHI, filling in with Klenow and religating to produce pSMTN2 and pSMTX2, respectively, as described in PCT/US96/09948 by Clackson et al. Examples of suitable packaging virus lines for preparing both ecotropic and amphotropic retroviral systems include Crip, Cre, 2 and Am.

Retroviruses, including lentiviruses, have been used to introduce a variety of genes into many different cell types, including neural cells, epithelial cells, retinal cells, endothelial cells, lymphocytes, myoblasts, hepatocytes, bone marrow cells, in vitro and/or in vivo (see for example, review by Federico (1999) Curr. Opin. Biotechnol. 10:448; Eglitis et al., (1985) Science 230:1395-1398; Danos and Mulligan, (1988) PNAS USA 85:6460-6464; Wilson et al., (1988) PNAS USA 85:3014-3018; Armentano et al., (1990) PNAS USA 87:6141-6145; Huber et al., (1991) PNAS USA 88:8039-8043; Ferry et al., (1991) PNAS USA 88:8377-8381; Chowdhury et al., (1991) Science 254:1802-1805; van Beusechem et al., (1992) PNAS USA 89:7640-7644; Kay et al., (1992) Human Gene Therapy 3:641-647; Dai et al., (1992) PNAS USA 89:10892-10895; Hwu et al., (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Furthermore, it has been shown that it is possible to limit the infection spectrum of retroviruses and consequently of retroviral-based vectors, by modifying the viral packaging proteins on the surface of the viral particle (see, for example PCT publications WO93/25234, WO94/06920, and WO94/11524). For instance, strategies for the modification of the infection spectrum of retroviral vectors include: coupling antibodies specific for cell surface antigens to the viral env protein (Roux et al., (1989) PNAS USA 86:9079-9083; Julan et al., (1992) J. Gen Virol 73:3251-3255; and Goud et al., (1983) Virology 163:251-254); or coupling cell surface ligands to the viral env proteins (Neda et al., (1991) J. Biol. Chem. 266:14143-14146). Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, and can also be used to convert an ecotropic vector in to an amphotropic vector.

E. Other Viral Systems

Other viral vector systems that can be used to deliver a polynucleotide of the invention have been derived from herpes virus, e.g., Herpes Simplex Virus (U.S. Pat. No. 5,631,236 by Woo et al., issued May 20, 1997 and WO 00/08191 by Neurovex), vaccinia virus (Ridgeway (1988) Ridgeway, “Mammalian expression vectors,” In: Rodriguez R L, Denhardt D T, ed. Vectors: A survey of molecular cloning vectors and their uses. Stoneham: Butterworth,; Baichwal and Sugden (1986) “Vectors for gene transfer derived from animal DNA viruses: Transient and stable expression of transferred genes,” In: Kucherlapati R, ed. Gene transfer. New York: Plenum Press; Coupar et al. (1988) Gene, 68:1-10), and several RNA viruses. Preferred viruses include an alphavirus, a poxivirus, an arena virus, a vaccinia virus, a polio virus, and the like. They offer several attractive features for various mammalian cells (Friedmann (1989) Science, 244:1275-1281 Ridgeway, 1988, supra; Baichwal and Sugden, 1986, supra; Coupar et al., 1988; Horwich et al.(1990) J.Virol., 64:642-650).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990, supra). This suggested that large portions of the genome could be replaced with foreign genetic material. The hepatotropism and persistence (integration) were particularly attractive properties for liver-directed gene transfer. Chang et al. recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al. (1991) Hepatology, 14:124A).

Since in certain embodiments, the compositions of the invention will be administered via a specific device, e.g., by injection using a syringe, the invention also provides devices, e.g., syringes, comprising a composition of the invention.

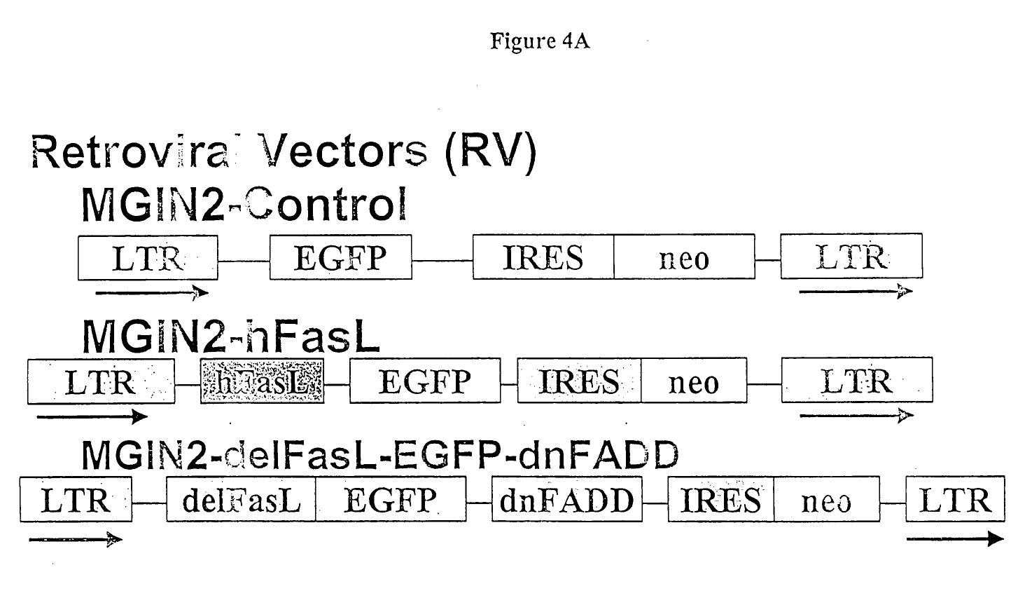

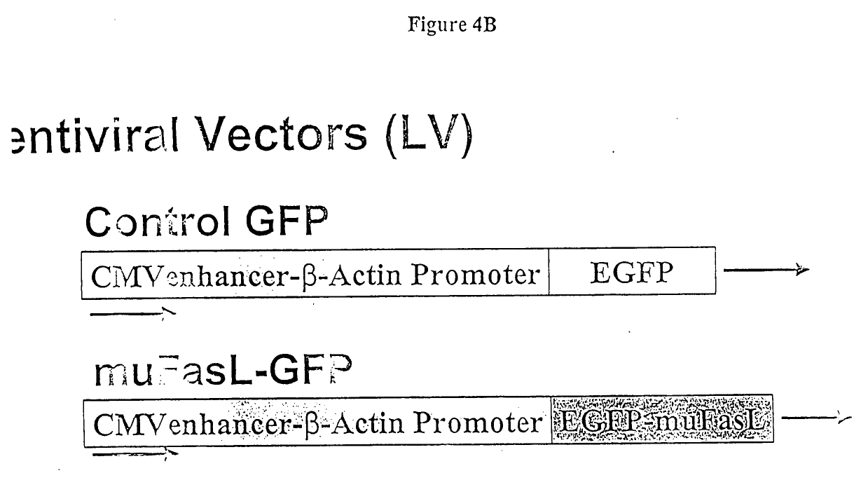

The preferred mammalian expression vectors include retrovirus and lentivirus-based expression vectors, such as those depicted in FIGS. 4A and 4B. Preferred mammalian vectors typically contain both prokaryotic sequences, to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papillomavirus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2^{nd} Ed., ed. by Sambrook, Fritsch and Maniatis (Cold Spring Harbor Laboratory Press: 1989) Chapters 16 and 17.

In some instances, it may be desirable to express the recombinant FASL polypeptide by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III)

When it is desirable to express only a portion of an FasL protein, such as a form lacking a portion of the N-terminus, i.e. a truncation mutant which lacks the signal peptide, it may be necessary to add a start codon (ATG) to the oligonucleotide fragment containing the desired sequence to be expressed. It is well known in the art that a methionine at the N-terminal position can be enzymatically cleaved by the use of the enzyme methionine aminopeptidase (MAP). MAP has been cloned from E. coli (Ben-Bassat et al. (1987) J. Bacteriol. 169:751-757) and Salmonella typhimurium and its in vitro activity has been demonstrated on recombinant proteins (Miller et al. (1987) PNAS 84:2718-1722). Therefore, removal of an N-terminal methionine, if desired, can be achieved either in vivo by expressing FasL derived polypeptides in a host which produces MAP (e.g., E. coli or CM89 or S. cerevisiae), or in vitro by use of purified MAP (e.g., procedure of Miller et al., supra).

Moreover, the gene constructs of the present invention can also be used as part of a gene therapy protocol to deliver nucleic acids encoding either an agonistic or antagonistic form of one of the subject FasL proteins. Thus, another aspect of the invention features expression vectors for in vivo or in vitro transfection and expression of an FasL polypeptide in particular cell types so as to reconstitute the function of, or alternatively, abrogate the function of FasL in a tissue. This could be desirable, for example, when the naturally-occurring form of the protein is misexpressed or the natural protein is mutated and less active.

In addition to viral transfer methods, non-viral methods can also be employed to cause expression of a subject FasL polypeptide in the tissue of an animal. Most nonviral methods of gene transfer rely on normal mechanisms used by mammalian cells for the uptake and intracellular transport of macromolecules. In preferred embodiments, non-viral targeting means of the present invention rely on endocytic pathways for the uptake of the subject FasL polypeptide gene by the targeted cell. Exemplary targeting means of this type include liposomal derived systems, poly-lysine conjugates, and artificial viral envelopes.

In other embodiments transgenic animals, described in more detail below could be used to produce recombinant proteins.

4.4. Polypeptides of the Present Invention

The present invention makes available isolated FasL polypeptides which are isolated from, or otherwise substantially free of other cellular proteins. The term “substantially free of other cellular proteins” (also referred to herein as “contaminating proteins”) or “substantially pure or purified preparations” are defined as encompassing preparations of FasL polypeptides having less than about 20% (by dry weight) contaminating protein, and preferably having less than about 5% contaminating protein. Functional forms of the subject polypeptides can be prepared, for the first time, as purified preparations by using a cloned gene as described herein.

Preferred FasL proteins of the invention have an amino acid sequence which is at least about 60%, 65%, 66%, 67%, 68%, 69%, 70%, 71%, 72%, 73%, 74%, 75%, 76%, 77%, 78%, 79%, 80%, 85%, 90%, or 95% identical or homologous to an amino acid sequence of a SEQ ID No. of the invention, such as a sequence shown in FIG. 8B (SEQ ID No. 2) or 9B (SEQ ID No. 4). Even more preferred FasL proteins comprise an amino acid sequence of at least 10, 20, 30, or 50 residues which is at least about 70, 80, 90, 95, 97, 98, or 99% homologous or identical to an amino acid sequence of a SEQ ID No. of the invention. Such proteins can be recombinant proteins, and can be, e.g., produced in vitro from nucleic acids comprising a nucleotide sequence set forth in FIG. 8A or 9A, or another nucleic acid SEQ ID No. of the invention or homologs thereof. For example, recombinant polypeptides preferred by the present invention can be encoded by a nucleic acid, which is at least 85% homologous and more preferably 90% homologous and most preferably 95% homologous with a nucleotide sequence set forth in a SEQ ID Nos. of the invention. Polypeptides which are encoded by a nucleic acid that is at least about 98-99% homologous with the sequence of a SEQ ID No. of the invention are also within the scope of the invention.

In a preferred embodiment, an FasL protein of the present invention is a mammalian FasL protein. In a particularly preferred embodiment an FasL protein is set forth as a SEQ ID No. of the invention. In particularly preferred embodiments, an FasL protein has an FasL bioactivity. It will be understood that certain post-translational modifications, e.g., phosphorylation and the like, can increase the apparent molecular weight of the FasL protein relative to the unmodified polypeptide chain.

The invention also features protein isoforms encoded by splice variants of the present invention. Such isoforms may have biological activities identical to or different from those possessed by the FasL proteins specified by a SEQ ID No. of the invention. Such isoforms may arise, for example, by alternative splicing of one or more FasL gene transcripts.

FasL polypeptides preferably are capable of functioning as either an agonist or antagonist of at least one biological activity of a wild-type (“authentic”) FasL protein of the appended sequence listing. The term “evolutionarily related to”, with respect to amino acid sequences of FasL proteins, refers to both polypeptides having amino acid sequences which have arisen naturally, and also to mutational variants of human FasL polypeptides which are derived, for example, by combinatorial mutagenesis.

Full length proteins or fragments corresponding to one or more particular motifs and/or domains or to arbitrary sizes, for example, at least 5, 10, 20, 25, 50, 75 and 100, amino acids in length are within the scope of the present invention.

For example, isolated FasL polypeptides can be encoded by all or a portion of a nucleic acid sequence shown in any of the sequences shown in FIG. 8B or 9B or a SEQ ID No. of the invention. Isolated peptidyl portions of FasL proteins can be obtained by screening peptides recombinantly produced from the corresponding fragment of the nucleic acid encoding such peptides. In addition, fragments can be chemically synthesized using techniques known in the art such as conventional Merrifield solid phase f-Moc or t-Boc chemistry. For example, an FasL polypeptide of the present invention may be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of a wild-type (e.g., “authentic”) FasL protein.

An FasL polypeptide can be a membrane bound form or a soluble form. A preferred soluble FasL polypeptide is a polypeptide which does not contain a hydrophobic signal sequence domain. Such proteins can be created by genetic engineering by methods known in the art. The solubility of a recombinant polypeptide may be increased by deletion of hydrophobic domains, such as predicted transmembrane domains, of the wild type protein.

In general, polypeptides referred to herein as having an activity (e.g., are “bioactive”) of a FasL protein are defined as polypeptides which include an amino acid sequence encoded by all or a portion of the nucleic acid sequences shown in one of the subject SEQ ID Nos. and which mimic or antagonize all or a portion of the biological/biochemical activities of a naturally occurring FasL protein. Examples of such biological activity include a region of conserved structure.

Other biological activities of the subject FasL proteins will be reasonably apparent to those skilled in the art. According to the present invention, a polypeptide has biological activity if it is a specific agonist or antagonist of a naturally-occurring form of an FasL protein.

Assays for determining whether a compound, e.g, a protein, such as an FasL protein or variant thereof, has one or more of the above biological activities include those assays, well known in the art, which are used for assessing FasL agonist and FasL antagonist activities.

Other preferred proteins of the invention are those encoded by the nucleic acids set forth in the section pertaining to nucleic acids of the invention. In particular, the invention provides fusion proteins, e.g., FasL-immunoglobulin fusion proteins. Such fusion proteins can provide, e.g., enhanced stability and solubility of FasL proteins and may thus be useful in therapy. Fusion proteins can also be used to produce an immunogenic fragment of an FasL protein. For example, the VP6 capsid protein of rotavirus can be used as an immunologic carrier protein for portions of the FasL polypeptide, either in the monomeric form or in the form of a viral particle. The nucleic acid sequences corresponding to the portion of a subject FasL protein to which antibodies are to be raised can be incorporated into a fusion gene construct which includes coding sequences for a late vaccinia virus structural protein to produce a set of recombinant viruses expressing fusion proteins comprising FasL epitopes as part of the virion. It has been demonstrated with the use of immunogenic fusion proteins utilizing the Hepatitis B surface antigen fusion proteins that recombinant Hepatitis B virions can be utilized in this role as well. Similarly, chimeric constructs coding for fusion proteins containing a portion of an FasL protein and the poliovirus capsid protein can be created to enhance immunogenicity of the set of polypeptide antigens (see, for example, EP Publication No: 0259149; and Evans et al. (1989) Nature 339:385; Huang et al. (1988) J. Virol. 62:3855; and Schlienger et al. (1992) J. Virol. 66:2).

The Multiple antigen peptide system for peptide-based immunization can also be utilized to generate an immunogen, wherein a desired portion of an FasL polypeptide is obtained directly from organo-chemical synthesis of the peptide onto an oligomeric branching lysine core (see, for example, Posnett et al. (1988) JBC 263:1719 and Nardelli et al. (1992) J. Immunol. 148:914). Antigenic determinants of FasL proteins can also be expressed and presented by bacterial cells.

In addition to utilizing fusion proteins to enhance immunogenicity, it is widely appreciated that fusion proteins can also facilitate the expression of proteins, and accordingly, can be used in the expression of the FasL polypeptides of the present invention. For example, FasL polypeptides can be generated as glutathione-S-transferase (GST-fusion) proteins. Such GST-fusion proteins can enable easy purification of the FasL polypeptide, as for example by the use of glutathione-derivatized matrices (see, for example, Current Protocols in Molecular Biology, eds. Ausubel et al. (N.Y.: John Wiley & Sons, 1991)). Additionally, fusion of FasL polypeptides to small epitope tags, such as the FLAG or hemagluttinin tag sequences, can be used to simplify immunological purification of the resulting recombinant polypeptide or to facilitate immunological detection in a cell or tissue sample. Fusion to the green fluorescent protein, and recombinant versions thereof which are known in the art and available commercially, may further be used to localize FasL polypeptides within living cells and tissue.

The present invention further pertains to methods of producing the subject FasL polypeptides. For example, a host cell transfected with a nucleic acid vector directing expression of a nucleotide sequence encoding the subject polypeptides can be cultured under appropriate conditions to allow expression of the peptide to occur. Suitable media for cell culture are well known in the art. The recombinant FasL polypeptide can be isolated from cell culture medium, host cells, or both using techniques known in the art for purifying proteins including ion-exchange chromatography, gel filtration chromatography, ultrafiltration, electrophoresis, and immunoaffinity purification with antibodies specific for such peptide. In a preferred embodiment, the recombinant FasL polypeptide is a fusion protein containing a domain which facilitates its purification, such as GST fusion protein.

Moreover, it will be generally appreciated that, under certain circumstances, it may be advantageous to provide homologs of one of the subject FasL polypeptides which function in a limited capacity as one of either an FasL agonist (mimetic) or an FasL antagonist, in order to promote or inhibit only a subset of the biological activities of the naturally-occurring form of the protein. Thus, specific biological effects can be elicited by treatment with a homolog of limited function, and with fewer side effects relative to treatment with agonists or antagonists which are directed to all of the biological activities of naturally occurring forms of FasL proteins.

Homologs of each of the subject FasL proteins can be generated by mutagenesis, such as by discrete point mutation(s), or by truncation. For instance, mutation can give rise to homologs which retain substantially the same, or merely a subset, of the biological activity of the FasL polypeptide from which it was derived. Alternatively, antagonistic forms of the protein can be generated which are able to inhibit the function of the naturally occurring form of the protein, such as by competitively binding to an FasL receptor.

The recombinant FasL polypeptides of the present invention also include homologs of the wildtype FasL proteins, such as versions of those protein which are resistant to proteolytic cleavage, as for example, due to mutations which alter ubiquitination or other enzymatic targeting associated with the protein.

FasL polypeptides may also be chemically modified to create FasL derivatives by forming covalent or aggregate conjugates with other chemical moieties, such as glycosyl groups, lipids, phosphate, acetyl groups and the like. Covalent derivatives of FasL proteins can be prepared by linking the chemical moieties to functional groups on amino acid sidechains of the protein or at the N-terminus or at the C-terminus of the polypeptide.

Modification of the structure of the subject FasL polypeptides can be for such purposes as enhancing therapeutic or prophylactic efficacy, stability (e.g., ex vivo shelf life and resistance to proteolytic degradation), or post-translational modifications (e.g., to alter phosphorylation pattern of protein). Such modified peptides, when designed to retain at least one activity of the naturally-occurring form of the protein, or to produce specific antagonists thereof, are considered functional equivalents of the FasL polypeptides described in more detail herein. Such modified peptides can be produced, for instance, by amino acid substitution, deletion, or addition. The substitutional variant may be a substituted conserved amino acid or a substituted non-conserved amino acid.

For example, it is reasonable to expect that an isolated replacement of a leucine with an isoleucine or valine, an aspartate with a glutamate, a threonine with a serine, or a similar replacement of an amino acid with a structurally related amino acid (i.e. isosteric and/or isoelectric mutations) will not have a major effect on the biological activity of the resulting molecule. Conservative replacements are those that take place within a family of amino acids that are related in their side chains. Genetically encoded amino acids can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine. (see, for example, Biochemistry, 2^{nd} ed., Ed. by L. Stryer, WH Freeman and Co.: 1981). Whether a change in the amino acid sequence of a peptide results in a functional FasL homolog (e.g., functional in the sense that the resulting polypeptide mimics or antagonizes the wild-type form) can be readily determined by assessing the ability of the variant peptide to produce a response in cells in a fashion similar to the wild-type protein, or competitively inhibit such a response. Polypeptides in which more than one replacement has taken place can readily be tested in the same manner.

This invention further contemplates a method for generating sets of combinatorial mutants of the subject FasL proteins as well as truncation mutants, and is especially useful for identifying potential variant sequences (e.g., homologs). The purpose of screening such combinatorial libraries is to generate, for example, novel FasL homologs which can act as either agonists or antagonist, or alternatively, possess novel activities all together. Thus, combinatorially-derived homologs can be generated to have an increased potency relative to a naturally occurring form of the protein.

In one embodiment, the variegated FasL libary of FasL variants is generated by combinatorial mutagenesis at the nucleic acid level, and is encoded by a variegated gene FasL library. For instance, a mixture of synthetic oligonucleotides can be enzymatically ligated into gene sequences such that the degenerate set of potential FasL sequences are expressible as individual polypeptides, or alternatively, as a set of larger fusion proteins (e.g., for phage display) containing the set of FasL sequences therein.

There are many ways by which such libraries of potential FasL homologs can be generated from a degenerate oligonucleotide sequence. Chemical synthesis of a degenerate gene sequence can be carried out in an automatic DNA synthesizer, and the synthetic genes then ligated into an appropriate expression vector. The purpose of a degenerate set of genes is to provide, in one mixture, all of the sequences encoding the desired set of potential FasL sequences. The synthesis of degenerate oligonucleotides is well known in the art (see for example, Narang, S A (1983) Tetrahedron 39:3; Itakura et al. (1981) Recombinant DNA, Proc 3^{rd} Cleveland Sympos. Macromolecules, ed. AG Walton, Amsterdam: Elsevier pp 273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

Likewise, a library of coding sequence fragments can be provided for an FasL clone in order to generate a variegated population of FasL fragments for screening and subsequent selection of bioactive fragments. A variety of techniques are known in the art for generating such 1, including chemical synthesis. In one embodiment, a library of coding sequence fragments can be generated by (i) treating a double stranded PCR fragment of an FasL coding sequence with a nuclease under conditions wherein nicking occurs only about once per molecule; (ii) denaturing the double stranded DNA; (iii) renaturing the DNA to form double stranded DNA which can include sense/antisense pairs from different nicked products; (iv) removing single stranded portions from reformed duplexes by treatment with S1 nuclease; and (v) ligating the resulting fragment library into an expression vector. By this exemplary method, an expression library can be derived which codes for N-terminal, C-terminal and internal fragments of various sizes.

A wide range of techniques are known in the art for screening gene products of combinatorial libraries made by point mutations or truncation, and for screening cDNA libraries for gene products having a certain property. Such techniques will be generally adaptable for rapid screening of the gene libraries generated by the combinatorial mutagenesis of FasL homologs. The most widely used techniques for screening large gene libraries typically comprises cloning the gene library into replicable expression vectors, transforming appropriate cells with the resulting libraries of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate FasL sequences created by combinatorial mutagenesis techniques. Combinatorial mutagenesis has a potential to generate very large libraries of mutant proteins, e.g., in the order of 1026 molecules. Combinatorial libraries of this size may be technically challenging to screen even with high throughput screening assays. To overcome this problem, a new technique has been developed recently, recrusive ensemble mutagenesis (REM), which allows one to avoid the very high proportion of non-functional proteins in a random library and simply enhances the frequency of functional proteins, thus decreasing the complexity required to achieve a useful sampling of sequence space. REM is an algorithm which enhances the frequency of functional mutants in a library when an appropriate selection or screening method is employed (Arkin and Yourvan, 1992, PNAS USA 89:7811-7815; Yourvan et al., 1992, Parallel Problem Solving from Nature, 2., In Maenner and Manderick, eds., Elsevir Publishing Co., Amsterdam, pp. 401-410; Delgrave et al., 1993, Protein Engineering 6(3):327-331).

The invention also provides for reduction of the FasL proteins to generate mimetics, e.g., peptide or non-peptide agents, such as small molecules, which are able to disrupt binding of an FasL polypeptide of the present invention with a molecule, e.g. target peptide. Thus, such mutagenic techniques as described above are also useful to map the determinants of the FasL proteins which participate in protein-protein interactions involved in, for example, binding of the subject FasL polypeptide to a target peptide. To illustrate, the critical residues of a subject FasL polypeptide which are involved in molecular recognition of its receptor can be determined and used to generate FasL derived peptidomimetics or small molecules which competitively inhibit binding of the authentic FasL protein with that moiety. By employing, for example, scanning mutagenesis to map the amino acid residues of the subject FasL proteins which are involved in binding other proteins, peptidomimetic compounds can be generated which mimic those residues of the FasL protein which facilitate the interaction. Such mimetics may then be used to interfere with the normal function of an FasL protein. For instance, non-hydrolyzable peptide analogs of such residues can be generated using benzodiazepine (e.g., see Freidinger et al. in Peptides: Chemistry and Biology, G. R. Marshall ed., ESCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988), substituted gamma lactam rings (Garvey et al. in Peptides: Chemistry and Biology, G. R. Marshall ed., ESCOM Publisher: Leiden, Netherlands, 1988), keto-methylene pseudopeptides (Ewenson et al. (1986) J Med Chem 29:295; and Ewenson et al. in Peptides: Structure and Function (Proceedings of the 9^{th} American Peptide Symposium) Pierce Chemical Co. Rockland, Ill., 1985), b-turn dipeptide cores (Nagai et al. (1985) Tetrahedron Lett 26:647; and Sato et al. (1986) J Chem Soc Perkin Trans 1:1231), and b-aminoalcohols (Gordon et al. (1985) Biochem Biophys Res Commun126:419; and Dann et al. (1986) Biochem Biophys Res Commun 134:71).

“Encoded by” refers to a nucleic acid sequence which codes for a polypeptide sequence, wherein the polypeptide sequence contains an amino acid sequence of at least 3 to 5 amino acids, more preferably at least 8 to 10 amino acids, and even more preferably at least 15 to 20 amino acids, a polypeptide encoded by the nucleic acid sequences. Also encompassed are polypeptide sequences which are immunologically identifiable with a polypeptide encoded by the sequence. Thus, a FasL “polypeptide,” “protein,” or “amino acid” sequence may have at least 60% similarity, preferably at least about 75% similarity, more preferably about 85% similarity, and most preferably about 95% similarity, to a polypeptide or amino acid sequence of a FasL. This amino acid sequence can be selected from the group consisting of the polypeptide sequence shown in FIG. 8B or 9B.

A “recombinant polypeptide” or “recombinant protein” or “polypeptide produced by recombinant techniques,” which are used interchangeably herein, describes a polypeptide which by virtue of its origin or manipulation is not associated with all or a portion of the polypeptide with which it is associated in nature and/or is linked to a polypeptide other than that to which it is linked in nature. A recombinant or encoded polypeptide or protein is not necessarily translated from a designated nucleic acid sequence. It also may be generated in any manner, including chemical synthesis or expression of a recombinant expression system.

The term “synthetic peptide” as used herein means a polymeric form of amino acids of any length, which may be chemically synthesized by methods well-known to the routineer. These synthetic peptides are useful in various applications.

The term “polynucleotide” as used herein means a polymeric form of nucleotides of any length, either ribonucleotides or deoxyribonucleotides. This term refers only to the primary structure of the molecule. Thus, the term includes double- and single-stranded DNA, as well as, double- and single-stranded RNA. It also includes modifications, such as methylation or capping, and unmodified forms of the polynucleotide. The terms “polynucleotide,” “oligomer,” “oligonucleotide,” and “oligo” are used interchangeably herein.

“A sequence corresponding to a cDNA” means that the sequence contains a polynucleotide sequence that is identical to or complementary to a sequence in the designated DNA. The degree (or “percent”) of identity or complementarity to the cDNA will be approximately 50% or greater, will preferably be at least about 70% or greater, and more preferably will be at least about 90%. The sequence that corresponds to the identified cDNA will be at least about 50 nucleotides in length, will preferably be about 60 nucleotides in length, and more preferably, will be at least about 70 nucleotides in length. The correspondence between the gene or gene fragment of interest and the cDNA can be determined by methods known in the art, and include, for example, a direct comparison of the sequenced material with the cDNAs described, or hybridization and digestion with single strand nucleases, followed by size determination of the digested fragments.

“Purified polynucleotide” refers to a polynucleotide of interest or fragment thereof which is essentially free, i.e., contains less than about 50%, preferably less than about 70%, and more preferably, less than about 90% of the protein with which the polynucleotide is naturally associated. Techniques for purifying polynucleotides of interest are well-known in the art and include, for example, disruption of the cell containing the polynucleotide with a chaotropic agent and separation of the polynucleotide(s) and proteins by ion-exchange chromatography, affinity chromatography and sedimentation according to density.

“Purified polypeptide” means a polypeptide of interest or fragment thereof which is essentially free, that is, contains less than about 50%, preferably less than about 70%, and more preferably, less than about 90% of cellular components with which the polypeptide of interest is naturally associated. Methods for purifying are known in the art.

The term “isolated” means that the material is removed from its original environment (e.g., the natural environment if it is naturally occurring). For example, a naturally-occurring polynucleotide or polypeptide present in a living animal is not isolated, but the same polynucleotide or DNA or polypeptide, which is separated from some or all of the coexisting materials in the natural system, is isolated. Such polynucleotide could be part of a vector and/or such polynucleotide or polypeptide could be part of a composition, and still be isolated in that the vector or composition is not part of its natural environment.

“Polypeptide” and “protein” are used interchangeably herein and indicates a molecular chain of amino acids linked through covalent and/or noncovalent bonds. The terms do not refer to a specific length of the product. Thus, peptides, oligopeptides and proteins are included within the definition of polypeptide. The terms include post-expression modifications of the polypeptide, for example, glycosylations, acetylations, phosphorylations and the like. In addition, protein fragments, analogs, mutated or variant proteins, fusion proteins and the like are included within the meaning of polypeptide.

A “fragment” of a specified polypeptide refers to an amino acid sequence which comprises at least about 3-5 amino acids, more preferably at least about 8-10 amino acids, and even more preferably at least about 15-20 amino acids, derived from the specified polypeptide.

“Recombinant host cells,” “host cells,” “cells,” “cell lines,” “cell cultures,” and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

As used herein “replicon” means any genetic element, such as a plasmid, a chromosome or a virus, that behaves as an autonomous unit of polynucleotide replication within a cell.

A “vector” is a replicon in which another polynucleotide segment is attached, such as to bring about the replication and/or expression of the attached segment.

The term “control sequence” refers to polynucleotide sequences which are necessary to effect the expression of coding sequences to which they are ligated. The nature of such control sequences differs depending upon the host organism. In prokaryotes, such control sequences generally include promoter, ribosomal binding site and terminators; in eukaryotes, such control sequences generally include promoters, terminators and, in some instances, enhancers. The term “control sequence” thus is intended to include at a minimum all components whose presence is necessary for expression, and also may include additional components whose presence is advantageous, for example, leader sequences.

“Operably linked” refers to a situation wherein the components described are in a relationship permitting them to function in their intended manner. Thus, for example, a control sequence “operably linked” to a coding sequence is ligated in such a manner that expression of the coding sequence is achieved under conditions compatible with the control sequences.

The term “open reading frame” or “ORF” refers to a region of a polynucleotide sequence which encodes a polypeptide; this region may represent a portion of a coding sequence or a total coding sequence.

A “coding sequence” is a polynucleotide sequence which is transcribed into mRNA and translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to, mRNA, cDNA, and recombinant polynucleotide sequences.

The term “immunologically identifiable with/as” refers to the presence of epitope(s) and polypeptide(s) which also are present in and are unique to the designated polypeptide(s). Immunological identity may be determined by antibody binding and/or competition in binding. These techniques are known to the routineer and also are described herein. The uniqueness of an epitope also can be determined by computer searches of known data banks, such as GenBank, for the polynucleotide sequences which encode the epitope, and by amino acid sequence comparisons with other known proteins.

As used herein, “epitope” means an antigenic determinant of a polypeptide. Conceivably, an epitope can comprise three amino acids in a spatial conformation which is unique to the epitope. Generally, an epitope consists of at least five such amino acids, and more usually, it consists of at least eight to ten amino acids. Methods of examining spatial conformation are known in the art and include, for example, x-ray crystallography and two-dimensional nuclear magnetic resonance.

A “conformational epitope” is an epitope that is comprised of specific juxtaposition of amino acids in an immunologically recognizable structure, such amino acids being present on the same polypeptide in a contiguous or non-contiguous order or present on different polypeptides.

A polypeptide is “immunologically reactive” with an antibody when it binds to an antibody due to antibody recognition of a specific epitope contained within the polypeptide. Immunological reactivity may be determined by antibody binding, more particularly by the kinetics of antibody binding, and/or by competition in binding using as competitor(s) a known polypeptide(s) containing an epitope against which the antibody is directed. The methods for determining whether a polypeptide is immunologically reactive with an antibody are known in the art.

As used herein, the term “immunogenic polypeptide containing an epitope of interest” means naturally occurring polypeptides of interest or fragments thereof, as well as polypeptides prepared by other means, for example, by chemical synthesis or the expression of the polypeptide in a recombinant organism.

The term “transformation” refers to the insertion of an exogenous polynucleotide into a host cell, irrespective of the method used for the insertion. For example, direct uptake, transduction or f-mating are included. The exogenous polynucleotide may be maintained as a non-integrated vector, for example, a plasmid, or alternatively, may be integrated into the host genome.

“Treatment” refers to prophylaxis and/or therapy.

The term “individual” as used herein refers to vertebrates, particularly members of the mammalian species and includes but is not limited to domestic animals, sports animals, primates and humans; more particularly the term refers to humans.

The term “sense strand” or “plus strand” (or “+”) as used herein denotes a nucleic acid that contains the sequence that encodes the polypeptide. The term “antisense strand” or “minus strand” (or “−”) denotes a nucleic acid that contains a sequence that is complementary to that of the “plus” strand.

The term “test sample” refers to a component of an individual's body which is the source of the analyte (such as, antibodies of interest or antigens of interest). These components are well known in the art. These test samples include biological samples which can be tested by the methods of the present invention described herein and include human and animal body fluids such as whole blood, serum, plasma, cerebrospinal fluid, urine, lymph fluids, and various external secretions of the respiratory, intestinal and genitorurinary tracts, tears, saliva, milk, white blood cells, myelomas and the like; biological fluids such as cell culture supernatants; fixed tissue specimens; and fixed cell specimens.

“Purified product” refers to a preparation of the product which has been isolated from the cellular constituents with which the product is normally associated, and from other types of cells which may be present in the sample of interest.

“PNA” denotes a “peptide nucleic acid analog” which may be utilized in a procedure such as an assay described herein to determine the presence of a target. “MA” denotes a “morpholino analog” which may be utilized in a procedure such as an assay described herein to determine the presence of a target. See, for example, U.S. Pat. No. 5,378,841, which is incorporated herein by reference. PNAs are neutrally charged moieties which can be directed against RNA targets or DNA. PNA probes used in assays in place of, for example, the DNA probes of the present invention, offer advantages not achievable when DNA probes are used. These advantages include manufacturability, large scale labeling, reproducibility, stability, insensitivity to changes in ionic strength and resistance to enzymatic degradation which is present in methods utilizing DNA or RNA. These PNAs can be labeled with such signal generating compounds as fluorescein, radionucleotides, chemiluminescent compounds, and the like. PNAs or other nucleic acid analogs such as MAs thus can be used in assay methods in place of DNA or RNA. Although assays arc described herein utilizing DNA probes, it is within the scope of the routineer that PNAs or MAs can be substituted for RNA or DNA with appropriate changes if and as needed in assay reagents.

“Analyte,” as used herein, is the substance to be detected which may be present in the test sample. The analyte can be any substance for which there exists a naturally occurring specific binding member (such as, an antibody), or for which a specific binding member can be prepared. Thus, an analyte is a substance that can bind to one or more specific binding members in an assay. “Analyte” also includes any antigenic substances, haptens, antibodies, and combinations thereof. As a member of a specific binding pair, the analyte can be detected by means of naturally occurring specific binding partners (pairs) such as the use of intrinsic factor protein as a member of a specific binding pair for the determination of Vitamin B12, the use of folate-binding protein to determine folic acid, or the use of a lectin as a member of a specific binding pair for the determination of a carbohydrate. The analyte can include a protein, a peptide, an amino acid, a nucleotide target, and the like.

“Inflammation” or “inflammatory disease,” as used herein, refer to infiltration of activated lymphocytes such as neutrophils, eosinophils, macrophages, T cells and B-cells, into a host tissue that results in damage to the host organism. Examples of inflammatory disease include but are not limited to conditions such as inflammatory bowel disease, sepsis, and rheumatoid arthritis.

An “Expressed Sequence Tag” or “EST” refers to the partial sequence of a cDNA insert which has been made by reverse transcription of mRNA extracted from a tissue, followed by insertion into a vector.

A “transcript image” refers to a table or list giving the quantitative distribution of ESTs in a library and represents the genes active in the tissue from which the library was made.

The present invention provides assays which utilize specific binding members. A “specific binding member,” as used herein, is a member of a specific binding pair. That is, two different molecules where one of the molecules through chemical or physical means specifically binds to the second molecule. Therefore, in addition to antigen and antibody specific binding pairs of common immunoassays, other specific binding pairs can include biotin and avidin, carbohydrates and lectins, complementary nucleotide sequences, effector and receptor molecules, cofactors and enzymes, enzyme inhibitors and enzymes, and the like. Furthermore, specific binding pairs can include members that are analogs of the original specific binding members, for example, an analyte-analog. Immunoreactive specific binding members include antigens, antigen fragments, antibodies and antibody fragments, both monoclonal and polyclonal, and complexes thereof, including those formed by recombinant DNA molecules.

The term “hapten,” as used herein, refers to a partial antigen or non-protein binding member which is capable of binding to an antibody, but which is not capable of eliciting antibody formation unless coupled to a carrier protein.

A “capture reagent,” as used herein, refers to an unlabeled specific binding member which is specific either for the analyte as in a sandwich assay, for the indicator reagent or analyte as in a competitive assay, or for an ancillary specific binding member, which itself is specific for the analyte, as in an indirect assay. The capture reagent can be directly or indirectly bound to a solid phase material before the performance of the assay or during the performance of the assay, thereby enabling the separation of immobilized complexes from the test sample.

The “indicator reagent” comprises a “signal-generating compound” (“label”) which is capable of generating and generates a measurable signal detectable by external means, conjugated (“attached”) to a specific binding member. “Specific binding member” as used herein means a member of a specific binding pair. That is, two different molecules where one of the molecules through chemical or physical means specifically binds to the second molecule. In addition to being an antibody member of a specific binding pair, the indicator reagent also can be a member of any specific binding pair, including either hapten-anti-hapten systems such as biotin or anti-biotin, avidin or biotin, a carbohydrate or a lectin, a complementary nucleotide sequence, an effector or a receptor molecule, an enzyme cofactor and an enzyme, an enzyme inhibitor or an enzyme, and the like. An immunoreactive specific binding member can be an antibody, an antigen, or an antibody/antigen complex that is capable of binding either to polypeptide of interest as in a sandwich assay, to the capture reagent as in a competitive assay, or to the ancillary specific binding member as in an indirect assay. When describing probes and probe assays, the term “reporter molecule” may be used. A reporter molecule comprises a signal generating compound as described hereinabove conjugated to a specific binding member of a specific binding pair, such as carbazol or adamantane.

4.5. Stem Cell Preparation and Manipulation

Methods for isolating and manipulating bone marrow cells, including hematopoietic stem cells, from a bone marrow graft donor are known in the art. For example, U.S. Pat. Nos. 4,965,204, 5,035,994, 5,081,030, 5,130,144, 5,137,809, 6,068,836 and 6,200,606, the contents of which patents are hereby incorporated by reference, describe methods for obtaining and manipulating bone marrow stem cells from a mammalian bone marrow donor. One of the most useful differentiation antigens for isolating human hematopoietic systems is the cell surface antigen known as CD34. CD34 is expressed by about 1% to 5% of normal human adult marrow cells in a developmentally, stage-specific manner (C I Civin et al., “Antigenic analysis of hematopoiesis. A hematopoietic progenitor cell surface antigen defined by a monoclonal antibody raised against KG-1a cells” J.Immunol., 133, 157-165, 1984). CD34+ cells are a mixture of immature blastic cells and a small percentage of mature, lineage-committed cells of the myeloid, erythroid and lymphoid series. Perhaps 1% of CD34+ cells are true HSC with the remaining number being committed to a particular lineage. Results in humans have demonstrated that CD34+ cells isolated from peripheral blood or marrow can reconstitute the entire hematopoietic system for a lifetime. Therefore, CD34 is a marker for HSC and hematopoietic progenitor cells.

For example, selective cytapheresis can be used to produce a cell suspension from human bone marrow or blood containing pluripotent lymphohematopoeitic stem cells. For example, marrow can be harvested from a donor (the patient in the case of an autologous transplant; a donor in the case of an allogenic transplant) by any appropriate means. The marrow can be processed as desired, depending mainly upon the use intended for the recovered cells. The suspension of marrow cells is allowed to physically contact, for example, a solid phase-linked monoclonal antibody that recognizes an antigen on the desired cells. The solid phase-linking can comprise, for instance, adsorbing the antibodies to a plastic, nitrocellulose or other surface. The antibodies can also be adsorbed on to the walls of the large pores (sufficiently large to permit flow-through of cells) of a hollow fiber membrane. Alternatively, the antibodies can be covalently linked to a surface or bead, such as Pharmacia Sepharose 6 MB macrobeads.RTM. The exact conditions and duration of incubation for the solid phase-linked antibodies with the marrow cell suspension will depend upon several factors specific to the system employed. The selection of appropriate conditions, however, is well within the skill of the art.

The unbound cells are then eluted or washed away with physiologic buffer after allowing sufficient time for the stem cells to be bound. The unbound marrow cells can be recovered and used for other purposes or discarded after appropriate testing has been done to ensure that the desired separation had been achieved. The bound cells are then separated from the solid phase by any appropriate method, depending mainly upon the nature of the solid phase and the antibody. For example, bound cells can be eluted from a plastic petrie dish by vigorous agitation. Alternatively, bound cells can be eluted by enzymatically “nicking” or digesting a enzyme-sensitive “spacer” sequence between the solid phase and the antibody. Spacers bound to agarose beads are commercially available from, for example, Pharmacia.

The eluted, enriched fraction of cells may then be washed with a buffer by centrifugation and either cryopreserved in a viable state for later use according to conventional technology or immediately infused intravenously into the transplant recipient.

In a particularly preferred embodiment, stem cells can be recovered directly from blood using essentially the above methodology. For example, blood can be withdrawn directly from the circulatory system of a donor and percolated continuously through a device (e.g., a column) containing the solid phase-linked monoclonal antibody to stem cells and the stem cell-depleted blood can be returned immediately to the donor's circulatory system using, for example, a conventional hemapheresis machine. When a sufficient volume of blood has been processed to allow the desired number of stem cells to bind to the column, the patient is disconnected. Such a method is extremely desirable because it allows rare peripheral blood stem cells to be harvested from a very large volume of blood, sparing the donor the expense and pain of harvesting bone marrow and the associated risks of anesthesia, analgesia, blood transfusion, and infection. The duration of aplasia for the transplant recipient following the marrow transplant can also be shortened since, theoretically, unlimited numbers of blood stem cells could be collected without significant risk to the donor.

The above methods of treating marrow or blood cell suspensions produce a suspension of human cells that contains pluripotent lympho-hematopoietic stem cells, but substantially free of mature lymphoid and myeloid cells. The cell suspension also contains substantially only cells that express the My-10 antigen and can restore the production of lymphoid and hematopoietic cells to a human patient that has lost the ability to produce such cells because of, for example, radiation treatment. By definition, a cell population that can restore the production of hematopoietic and lymphoid cells contains pluripotent lympho-hematopoietic stem cells.

The above cell populations containing human pluripotent lympho-hematopoetic stem cells can be used in therapeutic methods such as stem cell transplantation as well as others that are readily apparent to those of skill in the art. For example, such cell populations can be administered directly by I.V. to a patient requiring a bone marrow transplant in an amount sufficient to reconstitute the patient's hematopoietic and immune system. Precise, effective quantities can be readily determined by those skilled in the art and will depend, of course, upon the exact condition being treated by the therapy. In many applications, however, an amount containing approximately the same number of stem cells found in one-half to one liter of aspirated marrow should be adequate.

4.6. Dendritic Cell Preparation and Manipulation

Methods for isolating and manipulating dendritic cells, including dendritic antigen presenting cells from a bone marrow graft sample or from peripheral blood of a donor animal are known in the art. For example, U.S. Pat. Nos. 6,165,785 and 6,194,204, the contents of which patents are hereby incorporated by reference, describe methods for obtaining and manipulating dendritic (antigen-presenting) cells from a mammalian bone marrow donor.

One exemplary method for the enrichment of dendritic cells from the peripheral blood of a mammal utilizes the following steps. The mononuclear cells are separated from the peripheral blood. The mononuclear cells are separated into a first cell population having substantially lymphocytes and a second cell population having substantially myeloid cells. The myeloid cells are separated into a third cell population having substantially monocytes and a fourth cell population having substantially dendritic cells.

First, the mononuclear cells are separated from the peripheral blood. These mononuclear cells are separated into a first cell population having substantially lymphocytes and a second cell population having substantially myeloid cells. These myeloid cells are separated into a third cell population having substantially monocytes and a fourth cell population having substantially dendritic cells.

By peripheral blood is meant blood found in the circulation vasculature. The peripheral blood can be obtained from any mammal. By mammal is meant human as well as non-human mammal. Preferred non-human mammals is a mouse or a pig. Preferably, the peripheral blood is obtained from the same source from which the donor stem cell graft, e.g. the hematopoietic stem cell graft, is obtained. The mononuclear cells can be separated from the peripheral blood by any method known to those skilled in the art. Preferably, the method used does not affect cell function or viability. A preferred method is the use of centrifugation, preferably density gradient centrifugation, preferably discontinuous density gradient centrifugation. An alternative is the use of specific monoclonal antibodies.

The mononuclear cells are separated into a first cell population having substantially lymphocytes and a second cell population having substantially myeloid cells. Lymphocytes are meant to include, e.g., T cells, NK cells, B cells and mixtures thereof. By a cell population having substantially lymphocytes is meant that the cell population has greater than about 20% lymphocytes, preferably greater than about 40% lymphocytes, more preferably greater than about 60% lymphocytes, more preferably yet greater than about 80% lymphocytes, more preferably yet greater than about 90% lymphocytes, more preferably yet greater than about 95% lymphocytes, more preferably yet greater than about 98% lymphocytes, and most preferably greater than about 99% lymphocytes. Myeloid cells are meant to include monocytes and dendritic cells. Monocytes are also meant to include macrophages. It is known that monocytes circulate in the peripheral blood, and when they migrate to the tissue, they are called macrophages. This lineage of cells are commonly called monocyte/macrophage lineage. Myeloid cells are generally CD14.sup.+, CD33.sup.+ and CD13.sup.+. By a cell population having substantially myeloid cells is meant that the cell population has greater than about 20% myeloid cells, preferably greater than about 40% myeloid cells, more preferably greater than about 60% myeloid cells, more preferably yet greater than about 80% myeloid cells, more preferably yet greater than about 90% myeloid cells, more preferably yet greater than about 95% myeloid cells, more preferably yet greater than about 98% myeloid cells, and most preferably greater than about 99% myeloid cells.

In certain embodiments, the separation of the mononuclear cells into a first cell population having substantially lymphocytes and a second cell population having substantially myeloid cells comprises contacting the mononuclear cells with antibodies against the lymphocytes so as to form an antibody-lymphocyte complex, and selectively separating the antibody-lymphocyte complex from the myeloid cells. One or more than one type of antibody can be used. In certain embodiments, the contacting and the selectively separating steps are repeated. These steps can be repeated using the same type of antibody or antibodies against the lymphocytes, or they can be repeated using a different type of antibody or antibodies against the lymphocytes.

Both polyclonal and monoclonal antibodies can be used in this invention. Preferably, monoclonal antibodies are used. Antibodies against the lymphocytes include, e.g., T cell antibodies, NK cell antibodies, B cell antibodies, or mixtures thereof. Preferably, mixtures of the antibodies are used. The antibodies used are directed against one or more antigens which are expressed by one or more of the lymphocytes.

Preferably, the T cell antibodies are anti-CD3 antibodies. All T cells express the CD3 surface molecule. CD3 is described in Barclay et al., The Leukocyte Antigen Facts Book, Academic Press Limited (1993), pp. 106-109. Anti-CD3 antibodies can be obtained from Becton Dickinson Immunocytometry Systems, San Jose, Calif. or Coulter Corp., Miami, Fla. Other T cell antibodies that can be used include, e.g., anti-CD8 antibodies. CD8 is described in Barclay et al., The Leukocyte Antigen Facts Book, Academic Press Limited (1993), pp. 118-119. Anti-CD8 antibodies can be obtained from Becton Dickinson Immunocytometry Systems or Coulter Corp. Not all T cells express CD8. CD8 is expressed by roughly 40% of the T-lymphocyte population. Therefore, using, e.g., anti-CD8 antibodies will generally not result in the separation of the entire T cell population from the myeloid cells. There are, however, certain situations in which it might be desirable to use anti-CD8 antibodies. For example, CD.sup.8+ T lymphocytes represent a cytotoxic T-lymphocyte population. This population selectively targets and kills cells which were exposed to pathogen-specific antigens used in the production of pathogen-specific cytotoxic T cell lysis (intracellular pathogens).

Preferably, the NK cell antibodies are anti-CD16/56. CD16/56 refers to CD16 and CD56; they are not the same antigen, but are both expressed by NK cells. (CD8.sup.+ T lymphocytes also express CD16). Anti-CD16/56 antibodies can be obtained from Becton Dickinson Immunocytometry Systems or Coulter Corp. In certain embodiments, the NK cell antibodies can be anti-CD8. Not all NK cells express CD8, and therefore using anti-CD8 antibodies will not result in the separation of the entire NK cell population from the myeloid cells.

Preferably, the B cell antibodies are anti-CD19 or anti-CD20 antibodies. CD19 and CD20 are expressed by resting and activated B lymphocytes. CD19 and CD20 are described in Barclay et al., The Leukocyte Antigen Facts Book, Academic Press Limited (1993), pp. 142-143 and 144-145, respectively. Anti-CD19 and anti-CD20 antibodies can be obtained from Becton Dickinson Immunocytometry Systems or Coulter Corp.

The antibody-lymphocyte complex that is formed is selectively separated from the myeloid cells. In certain embodiments, this separation comprises contacting the antibody-lymphocyte complex and the myeloid cells with a matrix such that the antibody-lymphocyte complex is substantially retained by the matrix and the myeloid cells are substantially not retained by the matrix. Any matrix which performs such a separation can be used.

A matrix which is particularly useful is a mesh of steel wool which is inserted into a plastic column and placed in a magnetic field. A cell magnetic bead complex passes into the matrix and remains in the matrix as long as the column stays within the magnetic field. Examples of matrices include depletion columns type BS, type CS, type D RS+, and MS+ used for Mini Mags separator. (All these columns can be obtained from Miltenyi Biotec, Auburn, Calif.) Preferably, the matrix is provided in a column, though the matrix can be provided in any other way known to those skilled in the art, e.g., in a gel, on a filter, on a plate, on film or on paper.

By the complex being substantially retained by the matrix is meant that greater than about 20% of the complex is retained, preferably greater than about 40% is retained, more preferably greater than about 60% is retained, more preferably yet greater than about 80% is retained, more preferably yet greater than about 90% is retained, more preferably yet greater than about 95% is retained, and most preferably greater than about 98% is retained. By the myeloid cells being substantially not retained by the matrix is meant that greater than about 20% of the myeloid cells are not retained, preferably greater than about 40% are not retained, more preferably greater than about 60% are not retained, more preferably yet greater than about 80% are not retained, more preferably yet greater than about 90% are not retained, more preferably yet greater than about 95% are not retained, and most preferably greater than about 98% are not retained.

In preferred embodiments, the antibody-lymphocyte complex further comprises magnetic beads. Preferably, the magnetic beads are superparamagnetic microparticles, though any type of magnetic bead can be used. The magnetic beads can be attached, e.g., to the antibody or to the lymphocyte or to both. Preferably, the magnetic beads are attached to the antibody. Such attached antibodies can be obtained, e.g., from Miltenyi Biotec, Auburn, Calif. (as MACS superparamagnetic microbeads conjugated with monoclonal antibodies), or from Dynal Corp., Lake Success, N.Y. (as detachable or non-detachable large magnetic beads). See also Miltenyi et al., Cytometry 11:231-238 (1990). Preferably, the large magnetic beads (obtainable from Dynal Corp.), are used for the removal of lymphocytes. Preferably, the smaller beads (obtainable from Miltenyi Biotec), are used for the enrichment of the dendritic cells described below. The magnetic beads can be attached prior to the formation of the antibody-lymphocyte complex, or subsequent to the formation of the complex. Preferably, the magnetic beads are attached prior to formation of the complex.

In embodiments in which the antibody-lymphocyte complex has magnetic beads, separation of such a complex from the myeloid cells preferably comprises contacting the myeloid cells and the complex with a magnetic matrix such that the antibody-lymphocyte complex having the magnetic beads is substantially retained by the magnetic matrix and the myeloid cells are substantially not retained by the magnetic matrix. An example of a magnetic matrix is magnetized steel wool. Steel wool can be obtained from Miltenyi Biotec. The steel wool can be magnetized by, e.g., introducing it into a magnetic field, e.g., 0.6 Tesla, though other strength magnetic fields can also be used as known to those skilled in the art. The magnetic field can be produced, e.g., with a commercial electromagnet.

In certain embodiments, the antibodies to the T cells, NK cells and B cells are all contacted with the mononuclear cells prior to selectively separating the resulting antibody-lymphocyte complexes from the myeloid cells. In other embodiments, antibodies to only one type of lymphocyte cell are added (e.g., T cells), and the resulting antibody-lymphocyte complex is separated from the remaining cells. Antibodies to one of the remaining types of lymphocytes (e.g., NK cells) are then added to the remaining cells from above, and the resulting antibody-lymphocyte complex is separated from these remaining cells. Finally, antibodies to the remaining type of lymphocyte (e.g., B cells) are then added to this second batch of remaining cells, and the resulting antibody-lymphocyte complex is separated from these remaining cells (predominantly the myeloid cells). Preferably, all of the antibodies are added prior to selective separation.

The invention also includes embodiments in which separation of the mononuclear cells into a first cell population having substantially lymphocytes and a second cell population having substantially myeloid cells, comprises centrifugation. The centrifugation can be, e.g., density gradient centrifugation. For example, metrizamide 14.5% (obtained from Sigma Chemical Co., St. Louis, Mo.) or Monocyte 1 step (which is a pre-made discontinuous gradient which separates lymphocytes from myeloid cells, obtained from Accurate Chemical and Scientific Corp., Westbury, N.Y.), can be used. Centrifugation procedures are most useful if there are initially a large number of PBMCs, e.g., about 10.sup.9.

In certain embodiments, the separation of the mononuclear cells into a third cell population having substantially monocytes and a fourth cell population having substantially dendritic cells comprises contacting the myeloid cells with antibodies against the dendritic cells so as to form an antibody-dendritic cell complex, and selectively separating the antibody-dendritic cell complex from the monocytes. In certain embodiments, the contacting and the selectively separating steps are repeated. These steps can be repeated using the same type of antibody or antibodies against the dendritic cells, or they can be repeated using a different type of antibody or antibodies against the dendritic cells.

Preferably, monoclonal antibodies are used. The antibodies used are directed against one or more antigens which are expressed by the dendritic cells. Preferably, the antibodies are anti-CD2 antibodies, anti-CD5 antibodies, or mixtures thereof. Most preferably, anti-CD2 antibodies are used because they stain greater than 95% of the dendritic cells and do not modulate down in culture. Mixtures of the antibodies can also be used. CD2 and CD5 are described in Barclay et al., The Leukocyte Antigen Facts Book, Academic Press Limited (1993), pp. 104-105 and 112-113, respectively. Anti-CD2 antibodies can be obtained from Coulter Corp. Anti-CD5 antibodies can be obtained from Becton Dickinson Immunocytometry Systems or Coulter Corp.

The CD2 antigen is a 50 kD molecular weight glycoprotein that was initially identified on T cells and NK cells and has now been shown in this invention to be expressed by circulating dendritic cells. Antibodies to this surface antigen react strongly with resting T cells. The CD2 surface antigen is divided into three regions reflecting their functional relationship. The first region, T11.sub.1, is responsible for adhesion with the LFA-3 molecule and sheep erythrocyte binding. The first antibody that was produced to this region is called T11.sub.1 and its clone designation is 3PTH29. The second region, T11.sub.2, is an area on the CD2 antigen which does not interact with the binding domain but has been demonstrated to play a role in T cell activation in conjunction with a second antibody. The first antibody that was produced to this region is called T11.sub.2 and its clone designation is 1OLD24C1. Other T11.sub.2 clones are UMCD2/1E7E8, 0275, 9.6 and 7E10. The crosslinking of the T11.sub.2 region with monoclonal antibodies induces unfolding of the CD2 antigen and exposure of a cryptic epitope. This cryptic epitope represents a third region, T11.sub.3 or CD2R, and is expressed by activated T cells and cell-lines but only after exposure to T11.sub.2 monoclonal antibodies (or others with similar traits), which induces a conformational change in structure of the CD2 antigen. The first antibody to this region was T11.sub.3 and its clone name is 1 mono2A6. Other T11.sub.3 clones are VIT13, G144 and L304. In preferred embodiments, T11.sub.2 or T11.sub.2 plus T11.sub.3 antibodies are used.

In certain embodiments, prior to contacting the myeloid cells with antibodies, the myeloid cells are cultured, preferably for about 12 hours to about 36 hours, in about 5% to about 10% pooled mammal specific serum. For example, pooled human serum is used if the isolation is from human peripheral blood, and pooled pig serum is used if the isolation is from pig peripheral blood. After such culturing, antibodies, preferably anti-CD83 antibodies, can be used so as to form an antibody-dendritic cell complex. (CD83 is described in Zhou et al., J. Immunol. 154: 3821-3835 (1995); Crawford et al., Blood 80(10) Suppl. 1:192a (1992)). Anti-CD83 antibodies can be isolated as described in Zhou et al., J. Immunol. 149:735 (1992). The dendritic cells that are isolated in this embodiment can be phenotypically CD14.sup.-.

The antibody-dendritic cell complex that is formed, e.g., as a result of using any of the antibodies described above, is selectively separated from the monocytes. In certain embodiments, the separation comprises contacting the antibody-dendritic cell complex and the monocytes with a matrix such that the antibody-dendritic cell complex is substantially retained by the matrix and the monocytes are substantially not retained by the matrix. Preferably, the retained antibody-dendritic cell complex is then eluted from the matrix.

In preferred embodiments, the antibody-dendritic cell complex further comprises magnetic beads, as described above. In such embodiments, separation of the antibody-dendritic complex from the monocytes preferably comprises contacting the monocytes and antibody-dendritic cell complex having the magnetic beads with a magnetic matrix such that the antibody-dendritic cell complex having the magnetic beads is substantially retained by the magnetic matrix and the monocytes are substantially not retained by the magnetic matrix. Preferably, the retained antibody-dendritic cell complex is then eluted from the matrix. The complex can be eluted, e.g., by demagnetizing the matrix, e.g., by removing the matrix from the magnetic field.

Preferably, the dendritic cells in the fourth cell population are greater than about 60% pure, more preferably greater than about 70% pure, more preferably yet greater than about 80% pure, more preferably yet greater than about 90% pure, more preferably yet greater than about 95% pure, more preferably yet greater than about 98% pure, and most preferably greater than about 99% pure. In certain embodiments, the dendritic cells in the fourth cell population are substantially unactivated. In certain embodiments, the above method further comprises the step of activating the dendritic cells in the fourth cell population, comprising culturing the dendritic cells with T11.sub.3 antibodies or LFA-3 ligand.

Preferably, the monocytes in the third cell population are greater than about 70% pure, more preferably greater than about 80% pure, more preferably yet greater than about 90% pure, more preferably yet greater than about 95% pure, more preferably yet greater than about 98% pure, and most preferably greater than about 99% pure. Preferably, the monocytes in the third cell population are substantially unactivated. An advantage of the present invention is that it can produce monocytes which are unactivated. Other monocyte isolation procedures which use plastic adherence are known to rapidly induce monocyte activation. See Triglia et al., Blood 65(4):921-928 (1985).

The invention also includes a method for the enrichment of dendritic cells from the peripheral blood of a mammal comprising selecting cells from the peripheral blood which do not express antigens CD3, CD16/56 and CD19 or CD20, and which do express antigen CD2, CD5, CD83, or mixtures thereof. Preferably, cells are selected which also express antigen CD14. In certain embodiments, cells are selected which do not express antigen CD14.

The invention also includes a method for the enrichment of dendritic cells from tissue of a mammal. Tissue having mononuclear cells from a mammal is provided. The mononuclear cells are separated from the tissue. The mononuclear cells are separated into a first cell population having substantially lymphocytes and a second cell population having substantially myeloid cells. The myeloid cells are separated into a third cell population having substantially monocytes and a fourth cell population having substantially dendritic cells. The tissue can be from any part of the body of the mammal that has dendritic cells, e.g., skin or lymph nodes.

### 5. EXAMPLES

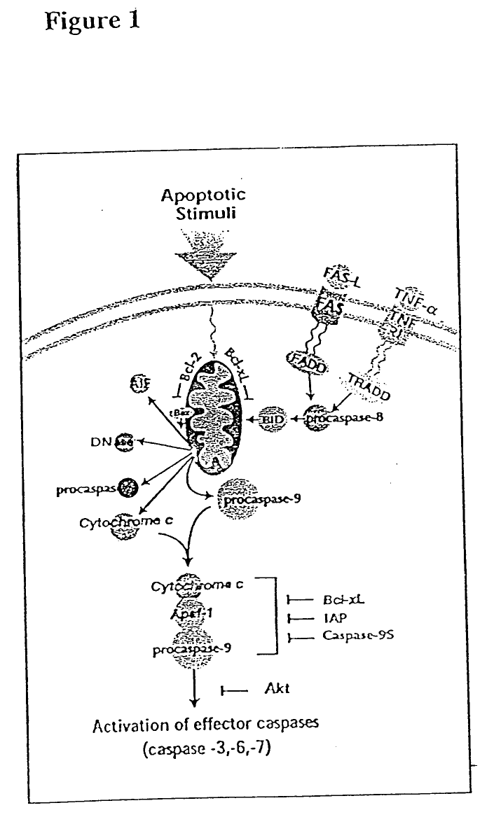

The examples below provide guidance to the skilled artisan in applying the methods and compositions of the invention to reducing the amount of non-specific immunosuppression required for stem cell engraftment by utilizing FasL to decrease the host T cell response against donor cells (HVG). The Fas/Fas L pathway (see FIG. 1) is an important physiologic mechanism by which activated T cells can be eliminated (see George et al. (1998) Nat Med 4: 333-35). Results from other systems show that dendritic cells (DQ genetically “armed” to express FasL kill cognate activated (Fas-expressing) T cells. Accordingly, administration of transduced FasL+ DC may be used to improve engraftment in allogeneic BMT. Further, hematopoietic stem cells (and/or very early progenitors; collectively abbreviated HSC) engineered to express FasL may be used to kill attacking T cells.

Allogeneic BMT is an important treatment option for many cases of leukemia, lymphoma and myeloma but is limited by complications. Transplant of rigorously isolated HSC should prevent GVHD, but in the absence of donor T cells, HVG rejection becomes a major problem.

Accordingly we engineered DC and HSC to constitutively express FasL in order to avoid this problem. We hypothesize that these FasL “armed” DC and HCS will function in vivo to kill anti-donor T cells, thus decreasing immune rejection of transplanted HSC and reducing or eliminating the need for preparative immunoablation and post-transplant pharmacologic immunosuppression of HVG.

### Example 1

### Manipulation of the Fas Pathway to Control Hematopoietic Graft Rejection

Upon FasL binding, cellular Fas oligomerizes and a cytoplasmic domain in Fas binds to FADD (Fas associated death domain), which triggers caspase-mediated apoptosis (see FIG. 1 and Green and Ware (1997) Proc Natl Acad Sci USA 94: 5986-90). The Fas pathway is important in regulating the immune response; for example, organ allograft rejection can be suppressed by FasL⁺ DC (see Min et al. (2000) J Immunol 164: 161-7). Activation upregulates Fas on T cells, targeting them for activation-induced apoptosis upon exposure to FasL (see Griffith & Ferguson (1997) Immunol Today 18: 240-44).

Experimental Approach

Using RV vectors, either DC or HSC cells are modified to express high FasL levels. FasL⁺ DC produce short-term donor cell tolerance, but not necessarily sufficiently potent or long-lasting donor cell tolerance to prevent rejection of the graft in the long term. Accordingly, a second approach would be to transplant FasL⁺ HSC, which provide greater intensity and duration of the effect on host anti-donor T cells. Persistent FasL expression in all progeny of FasL⁺ HSC may cause toxicity in vivo. Mouse models are used to investigate relevant immunobiology and toxicity in order to develop a FasL⁺ donor (mini-)BMT grafting strategy requiring less (or no) nonspecific host immunosuppression. One approach is to first tolerize the host using FasL+ DC, and then maintain anti-donor tolerance by transplant of a few FasL⁺ HSC mixed with untransduced HSC.

Functionality of FasL and dnFADD Constructs

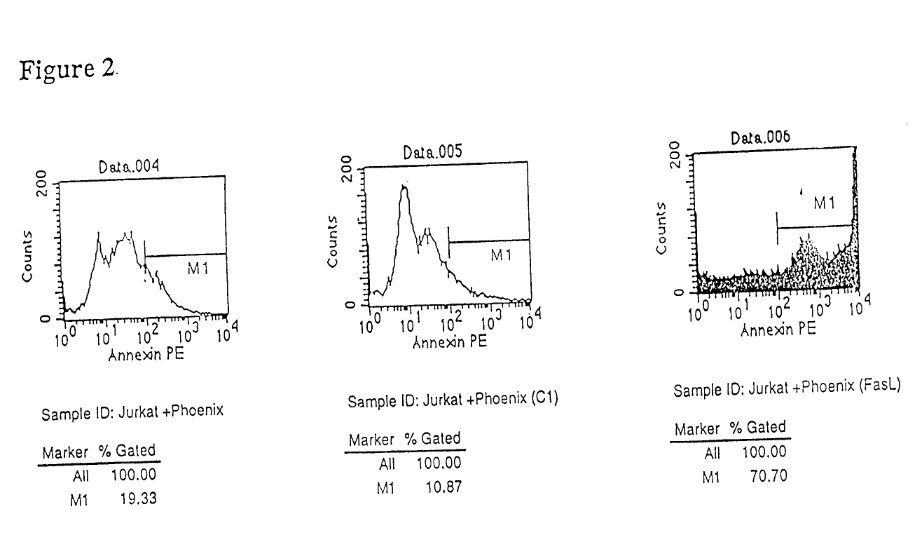

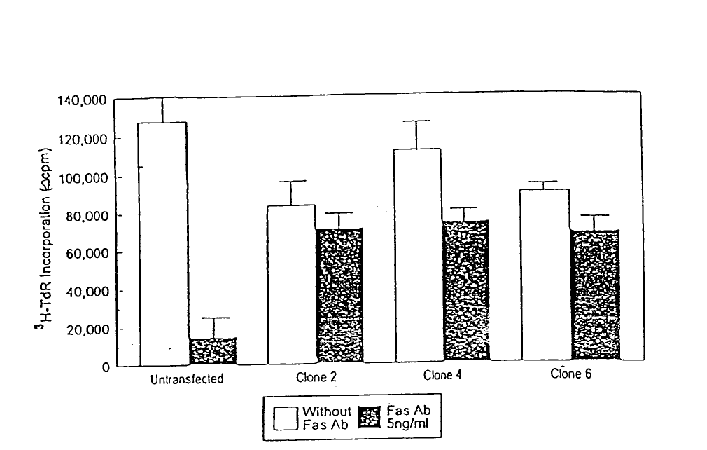

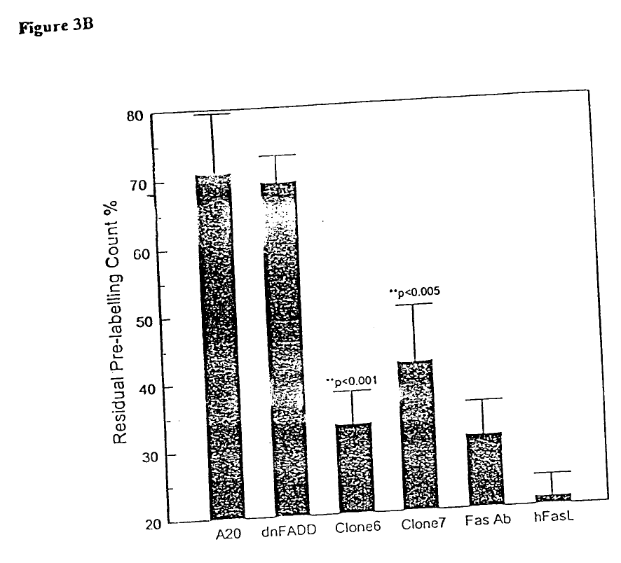

Transfection with FasL caused apoptosis of either human Jurkat or murine A20 lymphoid cells. Transfection with dnFADD protected A20 cells from Fas-mediated apoptosis (FIGS. 2 and 3).

Packaging Cell Lines Producing MGIN2-FasL

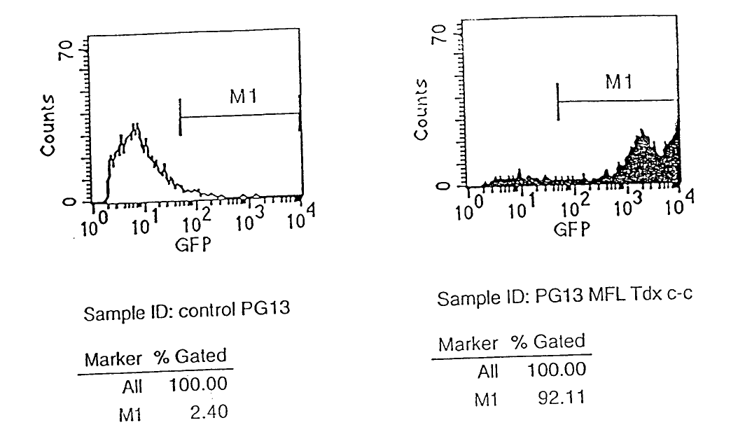

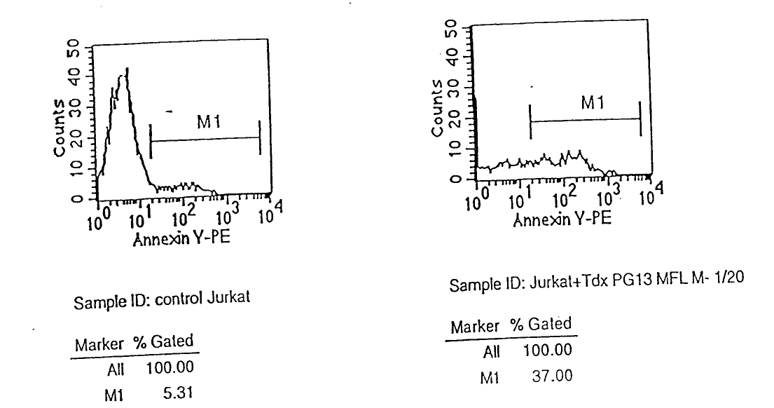

R V. MGIN2-FasL is our current murine stem cell RV expressing enhanced green fluorescent protein (GFP), NeoR and FasL (FIG. 4). Transduced PT67 cells were selected then FACS-sorted for intense GFP fluorescence. The resulting stable, high-producer PT67/MGIN2-FasL cells are used for transduction of murine cells. FasL on these cells was functional. PG13/MGIN2-FasL producer cells were prepared similarly, for transduction of human cells (FIG. 5).

MGIN2-FasL RV Cells Efficiently Transduced Murine Marrow and Human Cord Blood (CB) Progenitor Cells

Mouse marrow cells were transduced with PT67/MGIN2-FasL supernatant and assayed by hematopoietic colony-forming cell (CFQ assays (Table 1). Table 1A shows that the transduction procedure does not reduce total murine bone marrow progenitor cell numbers.

TABLE 1A

| Transduced Vector | CFC-Mix | CFC-GM | BFU-E |
|---|---|---|---|
| None (un-transduced) | | | |
| Mouse #1 | 53 ± 17 | 75 ± 12 | 20 ± 8 |
| Mouse #2 | 19 ± 2 | 46 ± 4 | 13 ± 7 |
| MGIN2 Control (FasL−) | | | |
| Mouse #1 | 39 ± 1 | 92 ± 12 | 12 ± 0 |
| Mouse #2 | 40 | 128 | 36 |
| MGIN2-FasL (FasL+) | | | |
| Mouse #1 | 30 ± 6 | 67 ± 13 | 31 ± 1 |
| Mouse #2 | 35 ± 11 | 74 ± 9 | 11 ± 4 |

CFC numbers (mean ± SEM) represent total CFC per 5 × 10⁴ marrow mononuclear cells plated.

Table 1B shows that RV from PT67/MGrN2-FasL cells transduce murine bone marrow progenitor cells and that the presence of FasL does not reduce the numbers of transduced (GFP+) CFC.

TABLE 1B

| | GFP + | GFP + | GFP + |
|---|---|---|---|
| Transduced Vector | CFC-Mix | CFC-GM | BFU-E |
| MGIN2 Control (FasL−) | | | |
| Mouse #1 | 4 ± 1 | 8 ± 1 | 2 ± 1 |
| Mouse #2 | 3 | 6 | 3 |
| MGIN2-FasL (FasL+) | | | |
| Mouse #1 | 4 ± 1 | 8 ± 3 | 6 ± 2 |
| Mouse #2 | 5 ± 2 | 10 ± 3 | 2 ± 1 |

CFC numbers represent GFP + CFC per 5 × 10⁴ marrow mononuclear cells plated, from the same experiment as in Table 1A.

Similar numbers of colonies and GFP⁺ colonies were observed after transduction with FasL⁺ vs parental control RV, suggesting that constitutive expression of FasL is not highly toxic to murine CFC. Human CB CD34⁺ cells were transduced with similar results (Table 2).

Table 2A shows that the transduction procedure does not reduce total human cord blood progenitor cell numbers.

TABLE 2A

| Transduced Vector | CFC-Mix | CFC-GM | BFU-E |
|---|---|---|---|
| None (un-transduced) | 18 ± 7 | 49 ± 8 | 24 ± 12 |
| MGIN2 Control (FasL−) | 22 ± 15 | 44 ± 12 | 30 ± 10 |
| MGIN2-FasL (FasL+) | 28 ± 5 | 47 ± 16 | 32 ± 8 |

CFC numbers (mean ± SEM) represent total CFC per 10³ cord blood CD34 + cells plated.

Table 2B shows that the V from PG 13/MGIN2-FasL cells transduce murine bone marrow progenitor cells; presence of FasL does not reduce the numbers of transduced (GFP+) CFC.

TABLE 2B

| | GFP + | GFP + | GFP + |
|---|---|---|---|
| Transduced Vector | CFC-Mix | CFC-GM | BFU-E |
| MGIN2 Control (FasL−) | 6 ± 3 | 6 ± 1 | 7 ± 4 |
| MGIN2-FasL (FasL+) | 17 ± 5 | 29 ± 12 | 12 ± 2 |

CFC numbers represent GFP + CFC per 10³ cord blood CD34 + cells plated, from the same experiment as in Table 2A.

Modification of the FasL Gene for Resistance to Cleavage

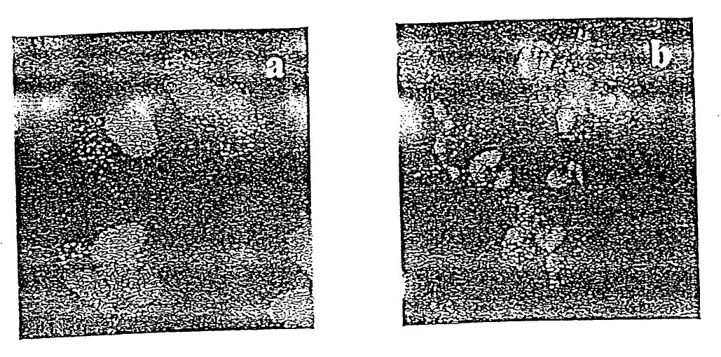

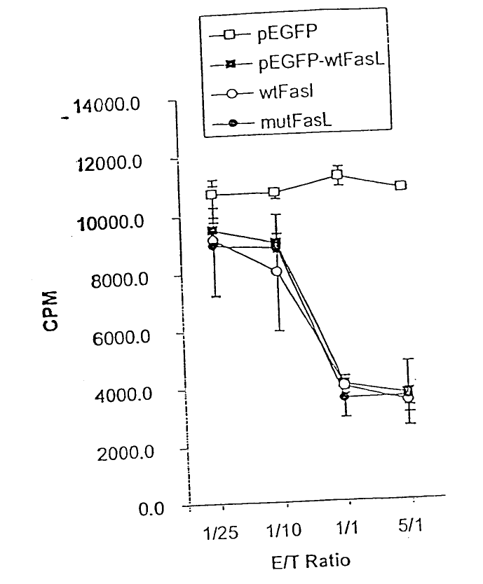

An unknown metalloprotease(s) cleaves membrane-bound FasL extracellularly to release a soluble moiety, which can bind Fas (Tanaka et al. (1996) Nat. Med. 2: 317-22). Since (a) cleavage would reduce the amount of cellular FasL and so decrease the potency of our FasL⁺ cells, and (b) soluble FasL might contribute to organ damage in vivo (Strasser et al. (1998) Nat. Med. 4: 21-22). We utilized functional delFasL (see e.g. FIG. 9, A and B), a non-cleavable truncated mutant of hFasL (Ayroldi et al. (1999) Blood 94: 3456-67). Then, we constructed a delFasL-GFP fusion gene, such that membrane fluorescence from GFP (FIG. 6) directly measures (covalently linked, non-cleavable) FasL expression, as we observed for a similar Nerve Growth Factor Receptor (NGFR-GFP) fusion (Yang et al. (1999) Am Soc Gene Therapy 65a).

FasL⁺ Balb1c Antigen Presenting Cells (APQ Reduced Specific T Cells, in a Transgenic Model

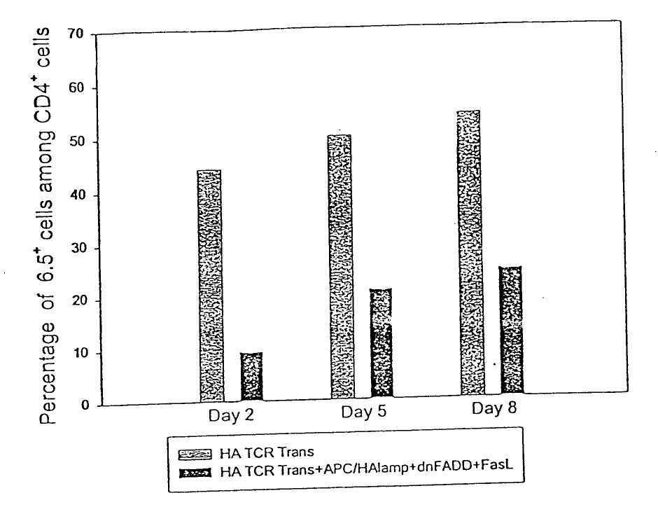

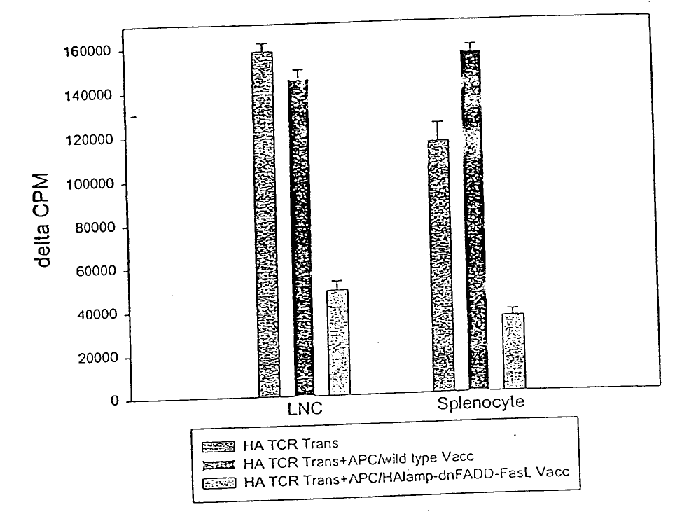

We infected Balb/c APC to express HA, dnFADD and hFasL. We administered these FasL⁺ Balb/c APC to HA-specific TCR transgenic mice, and observed a decrease in the numbers of anti-HA TCR transgenic T cells, as well as a decrease in T cell function (FIG. 7), confirming recent reports from 2 groups (Min et al. (2000) J Immunol 164: 161-7).

FasL⁺ DC will Delete Developing Anti-Allogeneic T Cells During Sensitization, Reducing the Alloimmune Response

DC are generated in ex vivo culture from Balb/c marrow cells18 and retrovirally transduced with FASL+ (i.e, our delFasL-GFP⁺/dnFADD⁺ RV, now being constructed (FIG. 4)) during the ex vivo culture (Novelli et al. (1999) Hum Gene Therap. 10: 2927-40). To show that the transduced DC express functional FASL+ and induce cellular apoptosis via Fas, we will first repeat the studies described in Prelim Results with Jurkat and A20 cells (If ongoing studies outside this project in our laboratories show that lentiviral vectors (LV) provide consistently superior gene transfer to DC (or HSC), we will, collaboratively, re-engineer our transgenes from RV to LV, and switch to the use of LV, throughout the Aim I (or 2) studies).

2C TCR Transgenic Mouse Model

The 2C transgenic model is based on a C57BL/6 (136) background mouse expressing TCR rearranged T cells specific for the H-2 Ld MHC I antigen (expressed on Balb/c cells) (Sha et al. (1988) Nature 335: 271-4). This 2C model provides a high anti-allogeneic response (>95% of CD8⁺ cells express the 2C TCR), which allows for sensitive detection of an effect of the FASL+ cells on alloimmune T cells (using the IB2 clonotypic Moab against the rearranged 2C TCR provided by D. Pardoll, Johns Hopkins Cancer Center).

In Vitro Experiments

We determine if FASL+ DC induce apoptosis in alloinimune CTL. 2C mice are allo-immunized to upregulate Fas expression, by iv injection of Balb/c splenocytes. After 1 wk, splenocytes from these allo-sensitized 2C mice (or unsensitized control 2C and B6 mice) are mixed with titered numbers of ex vivo generated DC of the following types: (1) FASL+ Balb/c DC, (2) FasL− Balb/c DC (transduced with the control NGFR-GFP⁺ RV), (3) FasL− Balb/c DC (untransduced control), (4) FasL+ B6 DC, or (5,6) FasL− B6 DC. After 4-36 hrs (as determined in preliminary experiments) incubation in vitro, we measure apoptosis of the sensitized 2C T cells by Annexin binding and flow cytometry, as in Prelim Results (The 2C cells are identified by labelling with I B2 Moab). We anticipate that Balb/c FASL+ DC will induce apoptosis of sensitized (Fas⁺) 2C T cells, while FasL− DC should not. B6 DC should induce no (or less) apoptosis, since alloantigen should be required. In similar in vitro systems, CTL have been specifically eliminated by FasL+ DC (see Min et al. (2000) J Immunol 164: 161-7). Since DC may be more potent if activated by TNF, CD40L or monocyte-conditioned medium Sauter et al. (2000) J Exp Med 191: 423-34), we will test the effects of these agents. We will also test the effect of FASL+ DC on T cells from allo-sensitized and unprimed (non-transgenic) B6 mice. These experiments will then be repeated using CTL from (B6) lpr mice—since lpr mice do not express FaS (van den Brink et al. (2000) J Immunol 164: 469-80), no effect of FASL+ DC should be observed.

In Vivo Experiments

We then assess the effect on allo-sensitized T cells of administration of FASL+ DC (or the above 5 controls) to mice. 2C mice are allo-sensitized as above, then titered doses of the above types of DC are administered iv. We then sacrifice mice twice weekly for 3 weeks, and measure the numbers of splenic 2C T cells. In similar studies (Min et al. (2000) J Immunol 164: 161-7; Zhang et al. (1998) Nat Biotech 16: 1045-9; Zhang et al. (1999) 162: 1423-30) (FIG. 7). CTL have been specifically eliminated by FasL⁺ DC. However, the response in 2C mice may be too strong to diminish detectably in our system. If so, we will adoptively transfer 2C cells to B6 mice (Sotomayer et al. (1999) Nat Med 5: 780-7) and generate mice with only 0.5-5% 2C T cells. One of these models will enable us to quickly determine the numbers of Balb/c FASL+ DC necessary for maximal killing of allo-sensitized T cells, as well as the effect of time of DC administration in relation to allo-aritigen priming. To confirm these results in a more physiological system, we will test the effect of FasL+ DC on T cells of allo-sensitized and unprimed B6 mice (and lpr mice, as a control). We will measure the (anticipated) decrease in allo-specific proliferation and CTL assays.

FasL⁺ DC Enhancse Engraftment of Allogeneic HSC

We utilize a model for allogeneic transplant of sublethally irradiated 2C recipient mice with Balb/c “HSC” (operationally defined as “Lin−” marrow cells purified using Stem Cell Technologies Cat #13066 immunomagnetic bead cocklail). We will first titrate (from 1-20×105 HSC) to determine the lowest number of Balb/c 14SC necessary to engraft (that is, generate mixed hematopoietic chimerism with 10-50% donor cells) in sublethally irradiated 2C mice (titering the dose down from. 700 cGy). Mice are sacrificcd at 1-6 months post-transplant, and engraftment of Balb/c HSC are determined by (a) flow cytometric quantification of the number of H2d⁺ cells in each of multiple lineages, and (b) donor CFC (by in situ immunostaining for H₂^{d}). This model should allow us to quantify the effect of graded doses of FasL+ DC on the levels of human lympho-hematopoietic cells, at fixed doses of HSC and irradiation. We will also be able to investigate whether co-transplant of FasL+ DC facilitates the same levels of engraftment with lower doses of HSC and/or irradiation.

In Vivo Evaluation of the Effect of FasL+ DC, Using the 2C Transplant Model

We co-transplant sublethally irradiated 2C mice with the above determined dose of HSC, along with 10⁵-10⁷ DC of the 6 types described above. Co-transplant of FasL+ Balb/C DC will increase the levels of donor cell engraftment at a given HSC dose, decrease the numbers of HSC needed to generate a given level of donor cell engraftment, and decrease the radiation dose required to attain a given level of donor cell engraftment. If DC are administered prior to HSC, the DC may begin to tolerize the recipient before the HSC transplant. Therefore, we determine whether administering DC prior to HSC allows for higher level engraftment (at lower HSC dose and/or with lower radiation doses) than simultaneous co-transplant of DC. We further test the effect of FasL⁺ DC on T cells of (non-transgenic) B6 transplant recipient mice (This will also serve as an alternative model system, if we cannot obtain engraftment of Balb/c HSC in 2C mice). These experiments are then be repeated in lpr recipient mice, where no effect of FasL+ DC should be observed.

Constitutive Expression of FasL in HSC Results in Suicide of HSC and/or Their Progeny; This Apoptosis May Be Prevented by Co-Expression of dnFADD

To show that FasL+ HSC induce cellular apoptosis, we test them on Jurkat and A20, as above. Then, we confirm the in vitro CFC capacity of FasL+ Balb/c HSC and FasL+ human CB CD34⁺ cells (vs the appropriate FasL− controls).

We then test the engrafting function of murine HSC in syngeneic (Balb/c) transplants, as well as that of human HSC in xenogeneic (NOD/SCID) transplants (Novelli et al. (1999) Hum Gene Ther 10: 2927-40; Leung et al. (1999) Blood and Marrow Transplant 5: 69-76). Quantitation of GFP⁺ murine progeny in multiple lineages in syngeneic transplanted mice tests the ability of FasL⁺ HSC to generate the repertoire of cell types in vivo, including DC, T, B, NK, megakaryocytic, monocytic, granulocytic, and erythroid cells (Only B, monocytic, granulocytic, and erytbroid in the more limited human model). In addition, transduced GFP⁺ donor cells are immunoaffinity isolated from syngeneic transplanted mouse marrow, and re-transplanted into naive irradiated syngeneic mice to assess 2° transplantation capacity as a rigorous test of their stem cell capacity (Civin et al. (1996) Blood 88: 4102-9). We also use the syngeneic transplanted mice to evaluate the toxicity (especially hepatic (Ogasawara et al. (1993) Nature 364: 806-9) and inflammatory (Arai et al. (1997) Proc Natl Acad. Sci, USA 94: 13862-7) of transplanted FasL+ HSC.

If FasL⁺ HSC are less efficient in CFC or engraftment assays (Schneider et al. (1999) Blood 94: 2613-21; Kang et al. (1997) Nat Med 3: 738-43; Barcena et al. (1999) Exp Hematol 27: 1428-39) then re-engineering the (human and/or murine) vectors to express higher levels of dnFADD, or as a second choice, lower levels of delFasL. Transduction of the physiologic Fas pathway antagonist, FLIP, may be another alternative (Perlman et al. (1999) J Exp Med 190: 1679-88).

FasL⁺ HSC Generate FasL⁺ DC In Vivo, which Should Delete Anti-Donor T Cells During Sensitization

We will transplant FasL+ Balb/c HSC into allo-immunized or un-primed 2C recipients, as above. At weekly intervals for 6 weeks post-transplant, cell suspensions are prepared from lymphoid organs, and transduced donor (GFP⁺) DC are enumerated and characterized for expression of HLA-DR, CD80, CD83 and CD86. We will also quantitate the numbers of 2C T cells. Finding abundant transduced DC in the thymus and massive reduction of 2C thymic cells (as measured by thymic weight and total numbers of thymic cells) might suggest that elimination of T cells occurs at the sensitization stage (McKay et al. (1999) J Immunol 163: 6455-61). We will repeat these transplants, using non-transgenic B6 and lpr recipients. If DC generated from FasL⁺ HSC appear to provide powerful deletion of anti-donor CTL, we will consider utilizing strategies to express FasL+ selectively in DC {Cheng and Pardoll, unpublished results). We would then test the effects of transplant of such transduced HSC, which might provide a potent, long-lasting tolerogenic effect, with low non-specific toxicity. If, long-lived allogeneic FasL+ DC (after they process and display antigens) kill T cells non-specifically in vivo, we will observe a generalized reduction of T cells, including T cells derived from the donor HSC. We would then determine whether their presence is necessary for permanent tolerance by deleting the FasL+ cells (at various timepoints after donor chimerism is achieved), using a Herpes Simplex thyrnidine kinase(TK)/ganciclovir approach (Boonini et al. (1997) Science 276: 1719-24). TK would be substituted for NeoR in the vector (see FIG. 4). Use of a conditional FasL fusion protein approach (Feil et al. (1996) Proc Natl Acad Sci USA 93: 108877-90) is an additional alternative.

FasL⁺ HSC and Their Progeny Will Delete Attacking Alto-Reactive Effetor T Cells in the Periphery

Even if FasL+ DC, alone, provide a sufficiently potent and specific cellular mechanism for killing anti-donor CTL, having the HSC themselves and all their progeny “armed” against anti-donor CTL may offer the advantage that HSC and progeny would kill CTL which might attack donor cells before DC-mediated tolerance is achieved, or which might evade the FasL+ DC. Since an “armed target” mechanism might be potent and specific, we will investigate this mechanism.

In Vitro Experiments

Allo-sensitized 2C T cells are mixed with FasL+ Balb/c HSC (vs the above controls) at a range of CTL:HSC ratios. After 4-36 hrs, apoptosis of 2C T cells are determined by the Annexin assay. The cell mixtures will also be plated for CFC. If FasL+ HSC kill allogeneic 2C T cells directly in vitro, the percents of apoptotic 2C T cells and surviving CFC will both be higher in mixtures containing FasL+ (vs control) HSC.

In Vivo Experiments

We will next determine whether administration of FasL+ HSC to mice will inhibit a response to alloantigen (These studies will not precisely discriminate the role that FasL+ HSC-de.rived DC play in the results, vs the armed target mechanism). We will sensitize 2C mice with Balb/c splenocytes, then transplant FasL+ or FasL− Balb/c (or B6) HSC. 3 days later, mice are sacrificed and clonotypic 2C T cells quantified. In addition, spleen cells are assessed by in vitro MLR to Balb/c stimulator cells and CTL against Balb/c target cells.

To determine whether a dose-response relation exists between the number of FasL+cells introduced and killing of allo-immune T cells, we will transfer graded doses of FasL+(vs control FasL−) Balb/c (or B6) cells to allo-immunized 2C mice and quantify (a) 2C T cell numbers, (b) GFP⁺ H2d⁺ donor cell numbers in multiple lineages, and (C)anti-Balb/c MLR and CTL responses, weekly after transplant. Key experiments are repeated in non-transgenic B6 and lpr mice.

FasL⁺ HS Engraft in Allogeneic Hosts

After transplant of Balb/c FasL+ (vs control) HSC to 2C mice, we will measure levels of multilineage donor cell engraftment, numbers of 2C T cells, and anti-Balb/c MLR and CTL responses, monthly for 6 months post-transplant. We- will repeat key experiments in B6 and lpr recipients. We will also observe (necropsy) whether murine allogeneic FasL+ HSC transplant produce organ toxicity in mice. Since in human mini-BMT, only a low radiation dose (200 cGy) plus pharmacologic immunosuppression is sufficient to allow mixed chimerism, no pharmacologic immunosuppression are used initially in the mouse model. As the radiation dose given to recipients is decreased, we will determine whether use of FasL+ HSC allows higher levels of donor cell engraftment at lower radiation and cell doses. The system can then be optimized to combine FasL delivery with a pbarmacologic mini-BMT regimen, as necessary. When long-lasting tolerance is achieved consistently, we will investigate deleting the FasL⁺ cells using a TK/ganciclovir (Boonini et al. (1997) Science 276: 1719-24), or a conditional fusion protein approach (Feil et al. (1996) Proc Natl Acad Sci USA 93: 108877-90).

Finally, we will use all of the above models to optimize these: approaches to HVG reduction by allo-tolerizing with FasL+ DC and/or HSC. We will attempt to achieve high levels of do)) or cell chimerism after allogeneic mini-transplant with the least FasL-mediated toxicity and the lowest requirement for non-specific radio-pharmacologic immunosuppression of HVG. Overcoming a fully allogeneic difference might be too formidable an objective; we may need to switch to a mouse transplant model involving smaller immunologic differences (haploidentical, etc). This proposal does not address suppression of GVH, which might be minimized by use- of highly purified HSC as the transplant graft, or reduction of potential “inherent” GVL, which might be replaced by the anti-cancer vaccine or donor lymphocyte infusion strategies developed outside of this project.

### Example 2

### Results of “Armed” Hematopoietic Cells to Reduce Graft Transplant Rejection

The goal of this application is to reduce or eliminate the non-specific immunosuppression required for stem cell engraftment, by utilizing FasL to decrease the host T cell response against donor cells (HVG). The Fas/Fas L pathway (FIG. 1) is an important physiologic mechanism in which activated T cells are killed. J. F. George et al., Nat. Med. 4,333-335 (1998); C. M. Eischen et al., J. Immunol. 159, 1135-1139 (1997); S. T. Ju et al., Nature 373, 444-448 (1995). Results from other systems show that dendritic cells (DCs) genetically “armed” to express FasL kill cognate activated T. W. P. Min et al., J. Immunol. 164, 161-167 (2000); H. Zhang et al., J. Immunol. 162, 1423-1430 (1999); H. G. Zhang et al., Nat. Biotech. 16, 1045-1049 (1998); B. Wu et al., Submitted, (2001);

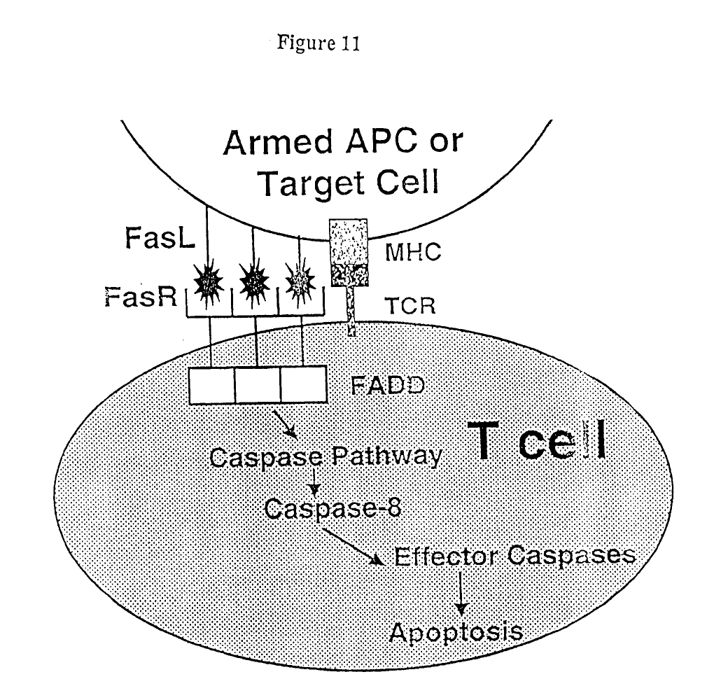

J.-M. Wu et al., Submitted, (2001). Thus, administration of transduced FasL⁺ DCs might improve engraftment in alloBMT. Further, hematopoietic stem cells (and/or very early progenitors; collectively abbreviated HSCs) and progeny engineered to express FasL would be expected to kill attacking T cells and further improve alloengraftment (FIG. 11).

In this study, we assess whether: FasL⁺ DCs specifically reduce an alloimmune response and enhance engraftment of allo HSCs; FasL⁺ DCs will delete anti-allo T cells during sensitization, reducing the alloimmune response; and whether administration of FasL⁺ DCs will enhance engraftment of transplanted allo HSCs. The overall goal is to demonstrate that FasL⁺ HSCs generate tolerance in allo transplantation.

Constitutive expression of FasL in HSCs may result in suicide of HSCs and/or their progeny. If so, this cytotoxicity could be overcome by co-expressing a dominant negative Fas pathway gene (dnFADD) to block Fas-mediated apoptosis in the FasL⁺ cells. FasL⁺ HSCs will generate in vivo FasL⁺ DC progeny, which should delete anti-donor T cells during sensitization. FasL⁺ HSCs and their progeny will delete attacking alloreactive effector T cells in the periphery. FasL⁺ HSCs will engraft in allo hosts.

Allographic Bone Marrow Transplantation (AlloBMT) is an important treatment option for many cases of leukemia, lymphoma and myeloma but is limited by complications. Rigorous isolation of transplanted HSCs will remove mature T cells and thereby prevent or reduce GVHD; but in the absence of donor T cells, HVG rejection becomes a major problem. We propose to manipulate DCs and HSCs to express FasL constitutively. We hypothesize that these cells and their progeny will function in vivo to kill anti-donor T cells, thus decreasing immune rejection of transplanted HSCs and reducing or eliminating the need for preparative immunoablation and post-transplant pharmacologic immunosuppression of HVG rejection, especially in allo NST.

The Fas Pathway in Immune Regulation

Upon FasL binding, cytoplasmic domains in the pre-formed cellular Fas trimer bind 3 FADD (Fas-associated death domain) molecules to form a DISC complex, which triggers caspase-mediated apoptosis. T. Suda et al., J. Immunol. 154, 3806-3813 (1995); S. Nagata, Cell 88, 355 (1997); D. R. Green, C. F. Ware, Proc. Natl. Acad. Sci. 94, 5986-5990 (1997); J. Wang et al., Eur. J. Immunol. 30, 155-163 (2000); R. M. Siegel et al., Science 288, 2354-2357 (2000).(FIG. 1). Recently, participation of the SADS molecule has been shown to enhance apoptosis. A. Suzuki et al., Nat Med 7, 88-93 (2001). Normally, activation of T cells targets them for apoptosis upon exposure to FasL. J. F. George et al., Nat. Med. 4, 333-335 (1998); C. M. Eischen et al., J. Immunol. 159, 1135-1139 (1997); S. T. Ju et al., Nature 373,444-448 (1995); T. S. Griffith, T. A. Ferguson, Immunol. Today 18, 240-244 (1997). Targeting T cells via the Fas pathway may be a feasible therapeutic strategy to regulate the immune response; eg, organ allograft rejection can be suppressed by FasL⁺ DCs. W. P. Min et al., J. Immunol. 164, 161-167 (2000); H. Zhang et al., J. Immunol. 162, 1423-1430 (1999); H. G. Zhang et al., Nat. Biotech. 16, 1045-1049 (1998).

Experimental Approach

Using (RV or) LV vectors, we will stably modify either DCs (Aim 1) or HSCs (Aim 2) to express high FasL levels. First we will investigate using FasL⁺ DCs, but FasL⁺ DCs might not produce sufficiently potent or long-lasting donor cell tolerance. Thus, our second approach will be to transplant FasL⁺ HSCs, which should provide greater intensity and duration of the effect on host anti-donor T cells. However, persistent FasL expression in all progeny of FasL⁺ HSCs may cause toxicity in vivo. In mouse models, we will investigate relevant immunobiology and toxicity in order to employ FasL⁺ donor cells in an alloNST strategy requiring less (or no) nonspecific host immunosuppression. One illustrative future approach might be to first tolerize the host using FasL⁺ DCs, and then maintain anti-donor tolerance by transplant of a few FasL⁺ HSCs mixed with untransduced HSCs.

LV Transduced FasL⁺ DCs and HSCs Kill Human and Murine T Cells

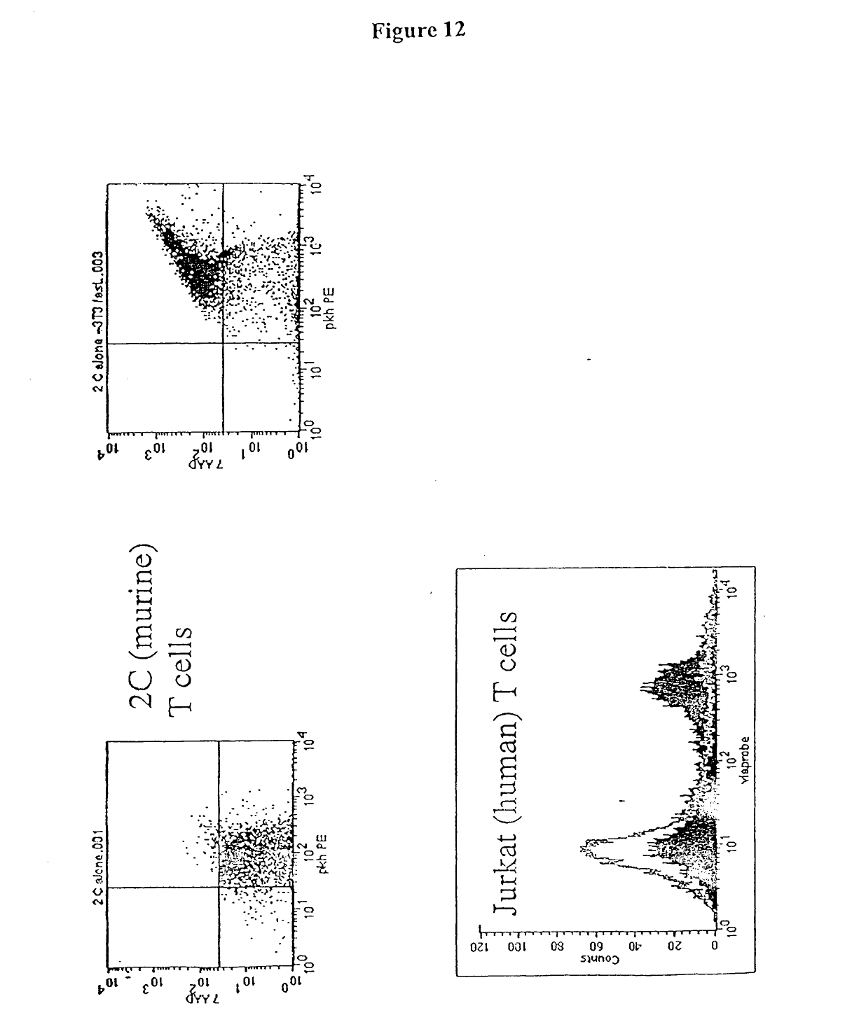

Preliminary experiments conducted with RV expressing human FasL demonstrated function of the FasL transgene, but only relatively low transduction efficiencies were observed, especially with murine cell targets. Our current LV vector expresses a fusion of the EGFP and murine FasL genes (FIG. 4A). This LV vector results in both higher transduction rates (up to 50%) and increased cellular EGFP signal. Using this vector, fluorescence from membrane-anchored EGFP directly reflects (covalently linked, non-cleavable) FasL expression, as we observed for a similar Nerve Growth Factor Receptor (NGFR-GFP) fusion. Y. Yang et al., Am. Soc. Gene Therapy, 65a (1999). LV-FasL⁺ transduced cells effectively kill Jurkat human T cells and murine 2C T cells, which are Fas sensitive targets (FIG. 12). The 2C transgenic model is based on a B6 background mouse expressing TCR rearranged T cells specific for a peptide derived from H-2 L^{d} MHC I antigen (expressed on BALB/c cells). W. C. Sha et al., Nature 335, 271-274 (1988). This 2C model provides a strong anti-allo response (>95% of CD8⁺ cells express the 2C TCR), which allows for sensitive detection of an effect of the FasL⁺ cells on the numbers of clonotypic alloimmune T cells. We use the anticlonotypic 1B2 MoAb, D. B. McKay et al., J. Immunol. 163, 6455-6461 (1999), to enumerate the clonotypic 2C T cells.

A ubiquitous metalloprotease(s) cleaves membrane-bound FasL extracellularly to release a soluble moiety, which can bind Fas. M. Tanaka et al., Nat. Med. 2, 317-22 (1996). Since (a) cleavage would reduce the amount of cellular FasL and so decrease the potency of our FasL⁺ cells, and (b) soluble FasL might contribute to organ damage in vivo, J. Ogasawara et al., Nature 364, 806-809 (1993); A. Strasser, L. O'Connor, Nat. Med. 4, 21-22 (1998), we generated functional delFasL, B. Wu et al., Submitted, (2001); J.-M. Wu et al., Submitted, (2001), a non-cleavable truncated mutant of hFasL. C. Perez et al., 63 2 (1990); E. Ayroldi et al., Blood 94, 3456-3467 (1999). We also have a functional dnFADD gene available, B. Wu et al., Submitted, (2001); J.-M. Wu et al., Submitted, (2001) (FIG. 4A).

FasL⁺ DCs Inhibit an Allogeneic Mixed Lymphocyte Reaction (MLR)

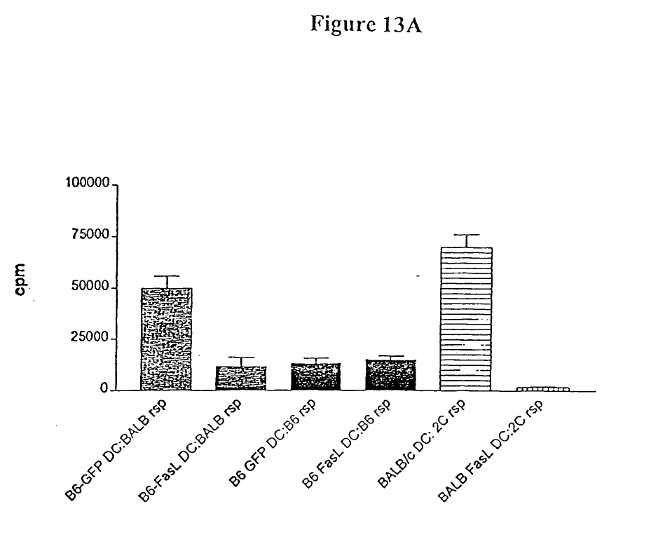

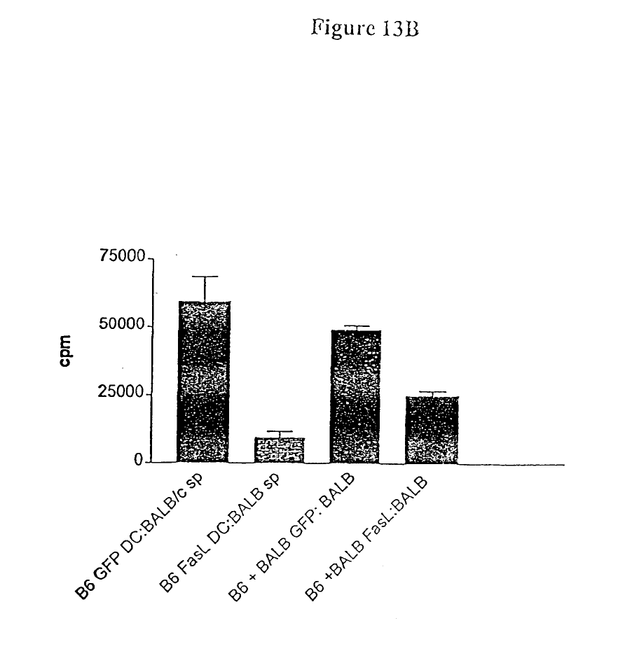

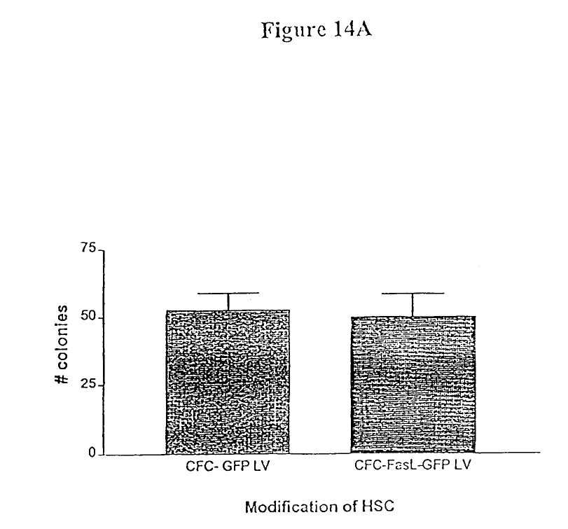

We conducted an MLR in which control or FasL⁺ C57BL/6 (B6) DCs were incubated with BALB/c splenocyte responders. The control DCs stimulated a robust MLR, while the response to FasL⁺ DCs was reduced to the background levels of the syngeneic response. BALB/c FasL⁺ DCs produced a similar decrease in proliferation in the 2C transgene model. In order to demonstrate that the inhibition was alloantigen specific we mixed control B6DCs with BALB/c FasL⁺ DCs. Expression of FasL on allo DCs is required for full inhibition of the MLR; ie, the decreased MLR was not completely due to a non-specific bystander effect that could be mediated by syngeneic FasL⁺ DCs (FIGS. 13A, B).

FasL is Not Toxic to Human or Murine Hematopoietic Cells

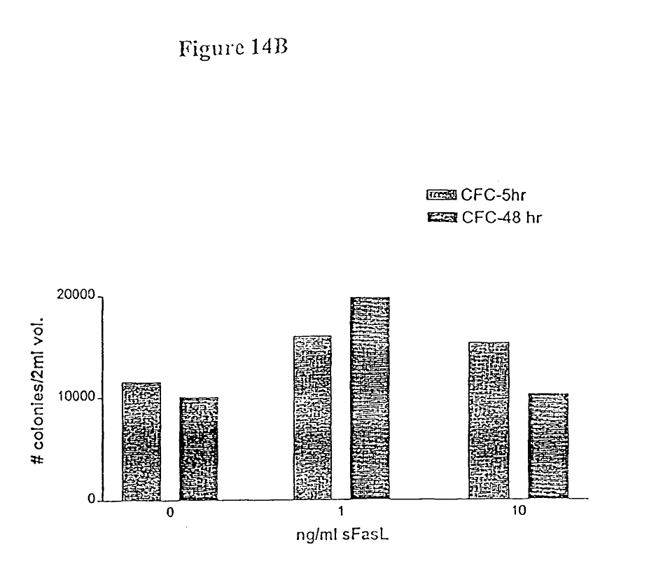

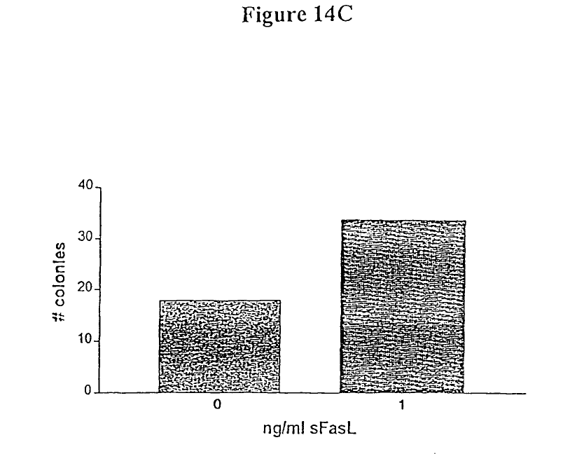

We first performed in vitro hematopoietic colony-forming cell (CFC) assays of cells exposed in culture to soluble FasL; no significant toxicity of treatment with high doses of soluble FasL was observed. Further, when human cells that had been treated with soluble FasL were transplanted to NOD/SCID mice, no effect on the levels or types of human cells engrafted were observed (data not shown). Finally, human or murine hematopoietic cells were transduced with FasL+ or the EGFP control LV. There was no significant effect of FasL transduction on the numbers or types of CFCs formed (FIGS. 14A, B, and C).

Human CD34⁺ Cells Contain FLIP

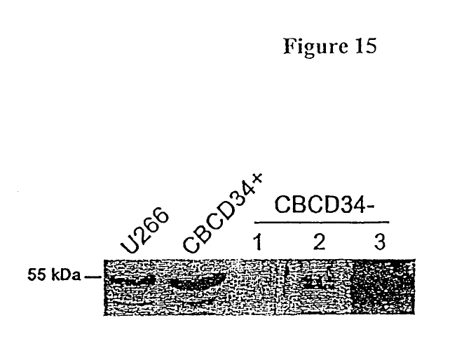

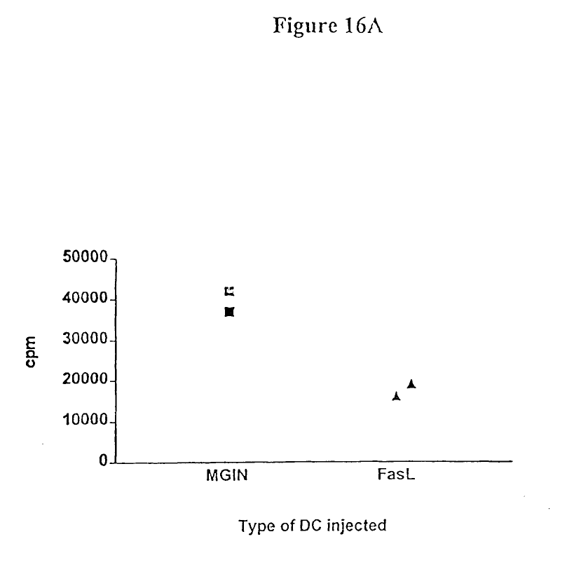

Purified human CD34⁺ cells expressed FLIP, (in much higher levels than CD34⁻ cells), which may be the mechanism protecting them from FasL induced apoptosis (FIG. 15).

Treatment of Mice with FasL⁺ Allo DCs Inhibits Alloreactivity and Enhances Engraftment

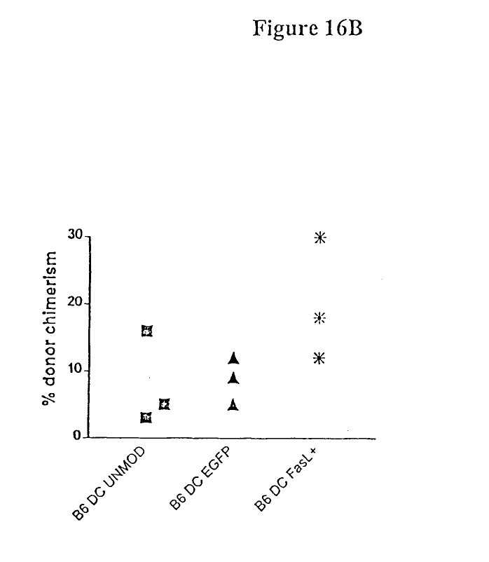

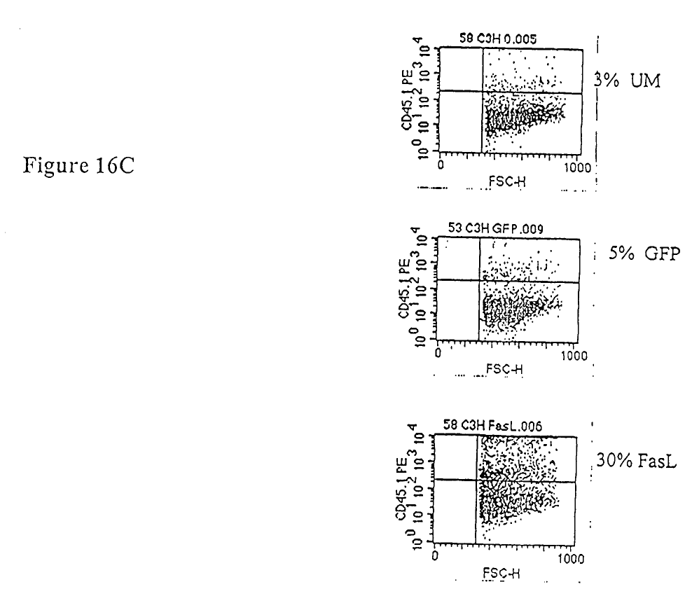

BALB/c mice were treated with a series of injections of control or FasL⁺ B6 DCs, then transplanted with unmodified B6 HSCs. 3 weeks later, responder splenocytes of the transplanted BALB/c mice were co-cultured with irradiated B6 splenocytes. This in vivo treatment of mice with FasL⁺ allo DCs reduced the allo MLR. (FIG. 16A). Unmodified, EGFP⁺, or FasL⁺ B6 DCs were injected into recipient C3H.SW mice for a series of 5 injections, followed by transplant of unmodified B6 bone marrow cells. Mice that had been treated with FasL⁺ DCs had higher levels of allo engraftment than either of the controls. MLRs on these mice and longer follow-up post BMT are pending (FIGS. 16B, C).

FasL⁺ HSC have Enhanced Engraftment

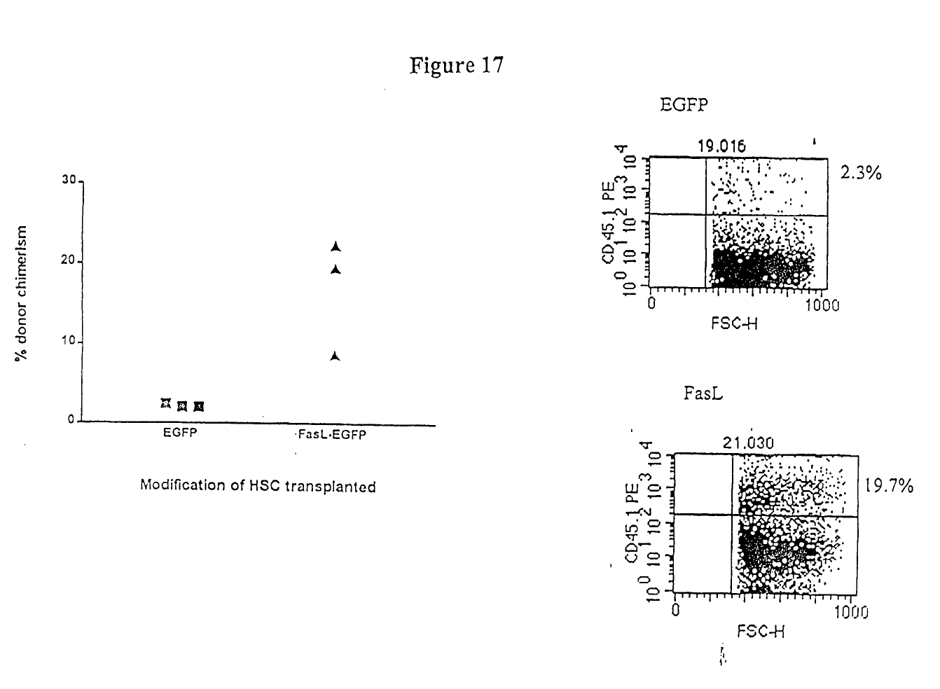

C3H.SW mice were irradiated (400 cGy) and transplanted with 10⁵ B6 HSC that were modified with either the control EGFP or FasL⁺ LV vector. 3 weeks later, mice were analyzed for the presence of donor cells. Mice that received FasL⁺ HSC had a significantly higher percentage of donor chimerism than those that received EGFP HSC (FIG. 17).

A first specific goal is to assess whether FasL⁺ DCs specifically reduce an alloimmune response and enhance HSC engraftment. FasL⁺ DCs will delete anti-allo T cells during sensitization, reducing the alloimmune response:

In vitro experiments: We determine if FasL⁺ DCs induce apoptosis of alloimmune CTL. Splenocytes from allosensitized 2C mice are be mixed with the following types of ex vivo generated DCs: (1) FasL⁺ BALB/c, (2) control EGFP⁺ BALB/c, (3) BALB/c (untransduced), (4) FasL⁺ B6, (5) EGFP⁺ B6, or (6) B6 (untransduced). After 4-36 hrs (as determined in prelim experiments) incubation in vitro, we measure apoptosis of the sensitized 2C T cells by flow cytometry. We anticipate that BALB/c FasL⁺ DCs induce apoptosis of sensitized 2C T cells, while FasL⁻ DCs should not. FasL⁺ B6 DCs should induce no (or less) apoptosis, since alloantigen should be required. In our own and other similar in vitro systems, CTLs have been specifically eliminated by FasL⁺ DCs. W. P. Min et al., J. Immunol. 164, 161-167 (2000); H. Zhang et al., J. Immunol. 162, 1423-1430 (1999); H. G. Zhang et al., Nat. Biotech. 16, 1045-1049 (1998). Since DCs may be more potent if activated by TNF, CD40L or monocyte-conditioned medium, B. Sauter et al., J. Exp. Med. 191, 423-434 (2000)], we test the effects of these agents. We also test the effect of FasL+ DCs on T cells from (non-transgenic) B6 mice. These experiments are then be repeated using CTLs from (B6) lpr mice—since lpr mice do not express Fas, M. R. M. van den Brink et al., J. Immunol. 164, 469-480 (2000), no effect of FasL+ DCs should be observed.

In vivo experiments: We then assess the effect on allosensitized T cells of administration of FasL⁺ BALB/c DCs (or the above 5 controls) to mice. 2C mice are allosensitized, then titered doses of DCs are administered. We then bleed mice twice weekly for 3 weeks to quantitate 2C T cells by FACS. After 3 weeks, we sacrifice the mice, and assess splenocytes, thymocytes, etc. In similar studies, W. P. Min et al., J. Immunol. 164, 161-167 (2000); H. Zhang et al., J. Immunol. 162, 1423-1430 (1999); H. G. Zhang et al., Nat. Biotech. 16, 1045-1049 (1998), and in Prelim Results (FIGS. 13,8), CTLs have been specifically eliminated by FasL+BALB/c DCs. However, the response in 2C mice may be too strong (ie too many alloreactive T cells) to diminish detectably in our system. If so, we adoptively transfer 2C cells to B6 mice[26] and generate mice with only 0.5-5% 2C T cells. 1 of these models enable us to quickly determine the numbers of BALB/c FasL⁺ DCs necessary for maximal killing of allosensitized T cells, as well as the effect of time of DC administration in relation to alloantigen priming. To confirm these results in a more physiological system, we test the effect of allo FasL⁺ DCs on T cells of B6 mice (and lpr mice, as a control). We measure the (anticipated) decrease in allospecific MLR and CTL assays.

FasL⁺ DCs Enhance Engraftment of Allo HSC

2C transplant model development: We develop a model for alloBMT of sublethally irradiated 2C recipient mice with BALB/c “HSCs” (operationally defined as “Lin-” marrow cells enriched using Stem Cell Technologies Cat# 13066 immunomagnetic bead cocktail). We first titrate (from 1-20×10⁵ HSCs) to determine the lowest number of BALB/c HSCs necessary to engraft (ie, generate mixed hematopoietic chimerism with 10-50% donor cells) in sublethally irradiated 2C mice (titering the radiation dose down from 700 cGy). Mice are bled at intervals, then sacrificed at 1-6 months post-transplant, and engraftment of BALB/c HSCs determined by (a) flow cytometric quantification of the number of H2^{d+} cells in each of multiple lineages (ie, myeloid, B, T, NK, DC, etc), and (b) donor CFCs (by in situ immunostaining for H₂^{d}, with the Pathology Core (F. Racke)). This model should allow us to quantify the effect of graded doses of FasL⁺ DCs on the levels of human lymphohematopoietic cells, at fixed doses of HSCs and irradiation. We are also able to investigate whether co-transplant of FasL⁺ DCs facilitates the same levels of engraftment with lower doses of HSCs and/or irradiation (FIG. 17).

In vivo evaluation of the effect of FasL⁺ DCs, using the 2C transplant model: We co-transplant sublethally irradiated 2C mice with the above determined dose of HSCs, along with 10⁵-10⁷ DCs of the 6 types described above. We anticipate that co-transplant of FasL⁺BALB/c DCs increase the levels of donor cell engraftment at a given HSC dose, decrease the numbers of HSCs needed to generate a given level of donor cell engraftment, and decrease the radiation dose required to attain a given level of donor cell engraftment (FIG. 17). If DCs are administered prior to HSCs, the DCs may begin to tolerize the recipient before the HSC transplant. Therefore, we determine whether administering DCs prior to HSCs allows for higher level engraftment (at lower HSC dose and/or with lower radiation doses) than simultaneous co-transplant of DCs. Our Prelim Results provide support for the hypothesis that engraftment be enhanced by FasL⁺ Dcs.

We also test the effect of FasL⁺ DCs on T cells of (non-transgenic) B6 transplant recipient mice (This also serves as an alternative model system, if we cannot obtain engraftment of BALB/c HSCs in 2C mice). These experiments are then repeated in lpr recipient mice, where no effect of FasL⁺ DCs should be observed.

Determining whether FasL⁺ HSCs Generate Tolerance in Allo Transplantation

Constitutive expression of FasL in HSCs might result in suicide of HSCs and/or their progeny. If observed, this apoptosis could be prevented by co-expression of dnFADD. We extend the Prelim Results testing in vitro CFC capacity of FasL⁺ murine HSCs and FasL⁺ human CD34⁺ cells (vs the appropriate FasL⁻ controls). We then test the engrafting function of murine HSCs in syngeneic murine transplants, as well as that of human HSCs in xenogeneic (NOD/SCID) transplants. W. Leung et al., Transplant. 68, 628-635 (1999); W. Leung et al., J. Invest. Med. 46, 303-311 (1998); E. M. Novelli et al., Hum. Gene Ther. 10, 2927-2940 (1999). Quantitation of GFP⁺ murine progeny in multiple lineages in syngeneic transplanted mice test the ability of FasL⁺ HSCs to generate the repertoire of cell types in vivo, including DC, T, B, NK, megakaryocytic, monocytic, granulocytic, and erythroid cells (Only B, monocytic, granulocytic, and erythroid in the more limited human model). In addition, transduced EGFP⁺ donor cells are immunoaffinity or FACS isolated from syngeneic transplanted mouse marrow, and re-transplanted into naive irradiated syngeneic mice to assess 2° transplantation capacity as a rigorous test of their stem cell capacity. C. I. Civin et al., Blood 88, 4102-4109 (1996); G. Guenechea et al., Mol Ther 1, 566-573 (2000). With the Pathology Core, we also use the syngeneic transplanted mice to evaluate the toxicity (especially hepatic J. Ogasawara et al., Nature 364, 806-809 (1993), and inflammatory, H. Arai et al., Proc. Natl. Acad. Sci. 94, 13862-13867 (1997); H. Lau, C. J. Stoeckert, Nat. Med. 3, 727-728 (1997); Y. Chen, J. M. Wilson, Nat. Biotech. 16, 1011-1012 (1998)) of transplanted FasL⁺ HSCs. If toxicity is observed, we test the use of the delFasL mutant (Prelim Results).

Both DCs and HSCs appear to be insensitive to FasL (FIGS. 14,7). DC are known to express the physiologic Fas pathway antagonist, FLIP, and our Prelim Results demonstrate FLIP in CD34⁺ cells (FIG. 15). Nevertheless, more extensive experiments may show that some subset of DCs, or HSCs or some type of progeny cells, are sensitive to FasL expression, and so must be protected, eg by dnFADD. If FasL⁺ HSCs are less efficient in repeated CFC or engraftment assays, E. Schneider et al., Blood 94, 2613-2621 (1999); S. M. Kang et al., Nat. Med. 3, 738-743 (1997); A. Barcena et al., Exp. Hematol. 27, 1428-1439 (1999), then we re-engineer the vector to express high levels of dnFADD (FIG. 4), or as a 2^{nd} choice, lower levels of FasL. Over-expression of FLIP is another alternative. J. Wang et al., Eur. J. Immunol. 30, 155-163 (2000); H. Perlman et al., J. Exp. Med. 190, 1679-1688 (1999). Finally, FasL expression in other cell types derived from HSC might be avoided by using a LV with a DC-specific promoter (developed in Project 4).

FasL⁺ HSCs Generate in Vivo FasL⁺ DC Progeny Which Should Delete Anti-Donor T Cells During Sensitization

We transplant FasL⁺ BALB/c HSCs into alloimmunized or unprimed 2C recipients, as above. 1-6 weeks post-transplant, cell suspensions be prepared from lymphoid organs, and transduced donor (EGFP+) DCs are enumerated and characterized for expression of DC markers (MHC Class II and CD86). We also quantitate the numbers of 2C T cells. Finding abundant transduced DCs in the thymus and massive reduction of 2C thymic cells (as measured by thymic weight, total numbers of thymic cells, with the presence of EGFP⁺/FasL⁺ cells in the thymus) might suggest that elimination of T cells occurs at the sensitization stage. D. B. McKay et al., J. Immunol. 163, 6455-6461 (1999). We repeat these transplants, using non-transgenic B6 and lpr recipients. If DCs generated from FasL⁺ HSCs appear to provide powerful deletion of anti-donor CTLs, we consider utilizing promoters and other Project 4 strategies to express FasL selectively in DCs. We would then test the effects of transplant of such transduced HSCs, which might provide a potent, long-lasting tolerogenic effect, with low nonspecific toxicity. If, long-lived allo FasL⁺ DCs (after they process and display antigens) kill T cells non-specifically in vivo, we observe a generalized reduction of T cells, including T cells derived from the donor HSCs. We would then determine whether the continued presence of FasL⁺ DCs is necessary for permanent tolerance by deleting the FasL⁺ cells (at various timepoints after donor chimerism is achieved), using a Herpes Simplex thymidine kinase(TK)/ganciclovir approach. C. Bonini et al., Science 276, 1719-1724 (1997). TK would be substituted for NeoR in the vector (FIG. 11). Use of a conditional FasL fusion protein approach, R. Feil et al., Proc. Natl. Acad. Sci. 93, 10887-10890 (1996), is an additional alternative.

FasL⁺ HSCs and Their Progeny Delete Attacking Alloreactive Effector T Cells in the Periphery

Even if FasL⁺ DCs, alone, provide a sufficiently potent and specific cellular mechanism for killing anti-donor CTLs, having the HSCs themselves and all their progeny “armed” against anti-donor CTLs may offer the advantage that HSCs and progeny would kill CTLs that might attack donor cells before DC mediated tolerance is achieved (or that might evade the FasL⁺ DCs). Since such an “armed target” mechanism might be potent and specific, we investigate this approach.

In vitro experiments: Allosensitized 2C T cells are mixed with FasL⁺ BALB/c HSCs (vs the above controls). Apoptosis of 2C T cells is determined by FACS. The cell mixtures are also be plated for CFCs. If FasL⁺ HSCs kill allo 2C T cells directly in vitro, the percents of apoptotic 2C T cells and surviving CFCs will both be higher in mixtures containing FasL⁺ (vs control) HSCs.

In vivo experiments: We next determine whether administration of FasL⁺ HSCs to mice inhibit an allo response (These studies do not precisely discriminate the role that FasL⁺ HSC-derived DCs play in the results, vs the armed target mechanism). We sensitize 2C mice with BALB/c splenocytes, then transplant FasL⁺ or FasL⁻ BALB/c (or B6) HSCs, followed by quantitation of clonotypic 2C T cells. In addition, spleen cells are assessed by in vitro MLR to BALB/c stimulator cells and CTL against BALB/c target cells.

To determine whether a dose-response relation exists between the number of FasL⁺ HSCs introduced and killing of alloimmune T cells, we transfer graded doses of FasL⁺ (vs control FasL⁻) BALB/c (or B6) cells to alloimmunized 2C mice and quantify (a) 2C T cell numbers, (b) GFP⁺ H2^{d+} donor cell numbers in multiple lineages, and (c)anti-BALB/c MLR and CTL responses, weekly after transplant. Key experiments are repeated in non-transgenic B6 and lpr mice.

FasL⁺ HSCs Engraft in Allo Hosts

After transplant of BALB/c FasL⁺ (vs control) HSCs to 2C mice, we measure levels of multilineage donor cell engraftment, numbers of 2C T cells, and anti-BALB/c MLR and CTL responses, monthly for 6 months post-transplant. We repeat key experiments in B6 and lpr recipients. We also observe (necropsy) whether murine FasL⁺ HSC allo transplants produce organ toxicity in mice. If organ toxicity is observed, we construct a LV containing a FasL deletion mutant that cannot be metalloprotease-cleaved to release soluble FasL (FIGS. 4A and B). This might reduce or eliminate organ toxicity. Since in human alloNST, only a low radiation dose (200 cGy) plus pharmacologic immunosuppression is sufficient to allow mixed chimerism, no pharmacologic immunosuppression will be used initially in the mouse model. As the radiation dose given to recipients is decreased, we determine whether use of FasL⁺ HSCs allows higher levels of donor cell engraftment at lower radiation and cell doses (FIG. 17). The system can then be optimized to combine FasL delivery with a pharmacologic NST regimen from Project 2, as necessary. When long-lasting tolerance is achieved consistently, we investigate deleting the FasL⁺ cells using a TK/ganciclovir, C. Bonini et al., Science 276, 1719-1724 (1997), or a conditional fusion protein approach. R. Feil et al., Proc. Natl. Acad. Sci. 93, 10887-10890 (1996).

Finally, we use all of the above models to optimize these approaches to HVG reduction by allotolerizing with FasL⁺ DCs and/or HSCs. We attempt to achieve high levels of donor cell chimerism after alloNST with the least FasL mediated toxicity and the lowest requirement for nonspecific radiopharmacologic immunosuppression of HVG (FIG. 4). Overcoming a full allo difference might be too formidable an objective; we may need to switch to a murine transplant model involving smaller immunologic differences (haploidentical, etc). In addition, the potency of FasL⁺ DCs and/or HSCs might be increased by co-expression of CD8, S. Reich-Zeliger et al., Immunity 13, 507-515 (2000), or a molecule recognizing a structure (eg, CD25) selectively expressed on activated T cells. This proposal does not address suppression of GVHD, which might be minimized by use of highly purified HSC as the transplant graft, or reduction of potential “inherent” GVL, which might be replaced by the more specific anti-cancer strategies.

### Example 3

### Further Studies

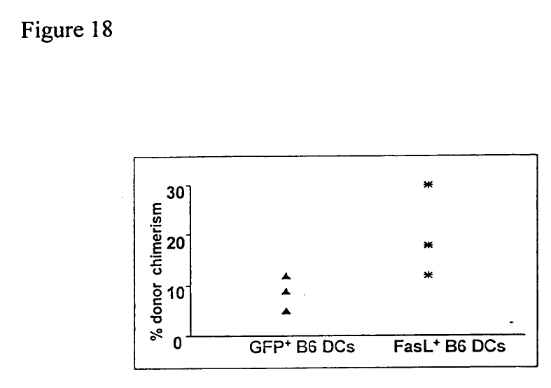

To test whether FasL DCs would enhance allo engraftment in an MHC-identical rather than a full haplotype mismatch BMT, a model was selected in which multiple minor mismatched donor HSCs were transplanted into recipients. C3H.SW mice were treated with a series of injections of control or FasL⁺ B6 DCs, followed by transplant of unmodified B6.SJL BM cells. Mice that had been treated with FasL⁺ DCs had somewhat higher levels of donor cells in blood than either of the controls (FIG. 18).

FIG. 18 shows in vivo treatment with FasL-transduced DCs enhanced allo engraftment. C3H.SW mice received 10⁵ B6.SJL DCs transduced with either GFP or FasL (5 injections, 3 days apart). 3 days after the last injection, mice were irradiated (400 cGy) and then transplanted with 10⁴ unmodified B6 HSCs. 3 wks later, blood was analyzed for donor (CD45.1⁺) cells by flow cytometry. Mice that had been treated with FasL⁺ DCs had a higher mean level of donor cells.

Constitutive FasL Expression by HSCs Does Not Impair Generation of CFCs

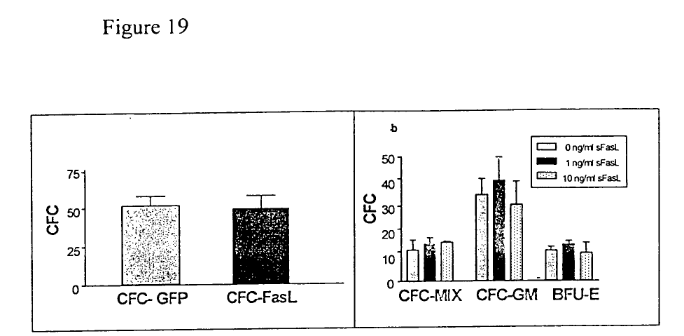

Mouse HSCs were transduced with either the GFP or FasL LV and plated in CFC assays. No significant difference was observed in numbers or types of CFCs from the 2 groups (FIG. 19A, see also FIG. 14A). In addition, culture of mouse HSCs in the presence of (oligomerized) sFasL did not inhibit CFC numbers or alter the distribution of CFC types (FIG. 19b). Similar results were obtained using human CD34⁺ cells in CFC and NOD/SCID engraftment assays.

FIG. 19 shows that FasL did not inhibit generation of CFCs from HSCs in vitro. B6.SJL HSCs were either (a) transduced with the GFP or FasL LV, or (b) exposed in culture to exogenously added sFasL (Alexis) for 48 hrs, then analyzed for CFCs. 3×10³ transduced HSCs were plated (triplicates) in methylcellulose media containing recombinant KL (50 ng/ml), IL-3 (10 ng/ml), GM-CSF (10 ng/ml) and erythropoietin (5 U/ml). CFC-Mix, CFC-GM and BFU-E colonies were counted. The results with the transduced cells are (a) the averages (±SEM) of 4 experiments, and the results with sFasL are (b) the averages of 2 experiments.

Human CD34⁺ Cells Contain FLIP

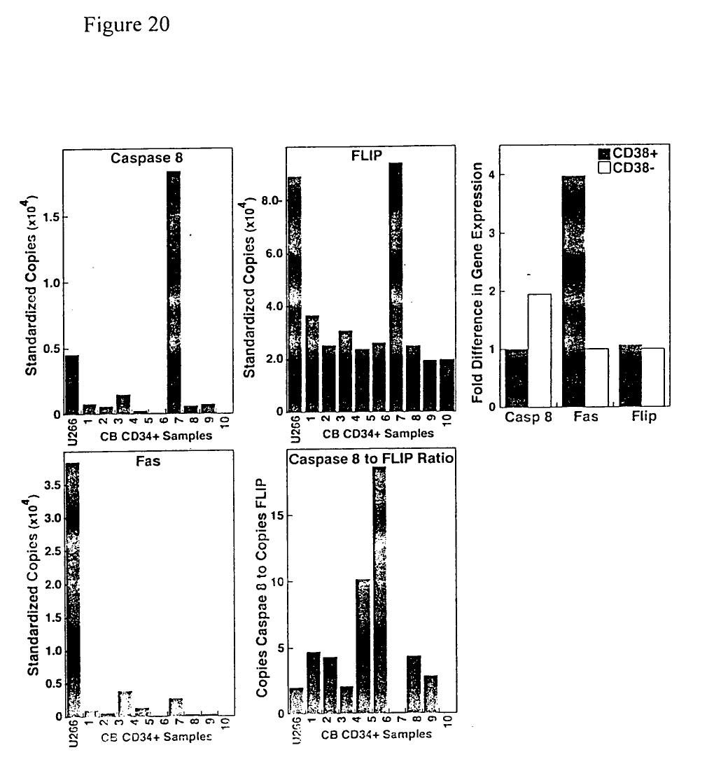

Purified human CD34⁺ and CD34⁺/CD38⁻ cells expressed high levels of the Fas pathway inhibitor, FLIP, in much higher levels than in CD34⁻ cells This may be 1 of the mechanisms protecting them from FasL-induced apoptosis (FIGS. 20A, B).

FIG. 20 shows that CD34⁺ and CD34⁺/CD38⁻ cells may be protected from Fas-mediated apoptosis by high levels of FLIP. Total RNA was isolated from Jurkat, U266, PBSC CD34⁺, and CB CD34⁺ cells. After poly-A mRNA reverse transcription, the number of copies of Caspase 8, Fas, and FLIP message were quantified by real-time quantitative PCR (qPCR) from 1 ng samples of total RNA (˜10³ cells). Fas expression varied among the different cell types, with Jurkat cells (5×10⁵ copies) expressing ˜10 times as much Fas mRNA as U266 cells (7×10⁴ copies). PBSC CD34⁺(2×10⁴) and CD CD34⁺ (7×10³) cells expressed Fas mRNA, but at levels much lower than in the control cell lines. PBSC CD34⁺ cells (10⁵ copies) and CB CD34⁺ cells (7×10⁴ copies) expressed levels of Caspase 8 mRNA, comparable to that of U266 cells (4×10⁵ copies), but ˜10-fold lower than in Jurkat cells (4×10⁶ copies). U266 (4×10⁴ copies), PBSC CD34⁺ (5×10⁴ copies), and CB CD34⁺ (1×10⁴ copies) cells expressed FLIP mRNA at comparable, high levels, ˜100 times higher than in Jurkat cells (8×10² copies). The ratio of Caspase 8:FLIP was 5255:1 for Jurkat cells, 10:1 for U266 cells, 2:1 for PBSC CD34⁺ cells, and 5:1 for CB CD34⁺ cells.

Mouse FasL⁺ HSCs Enhanced Allo Engraftment

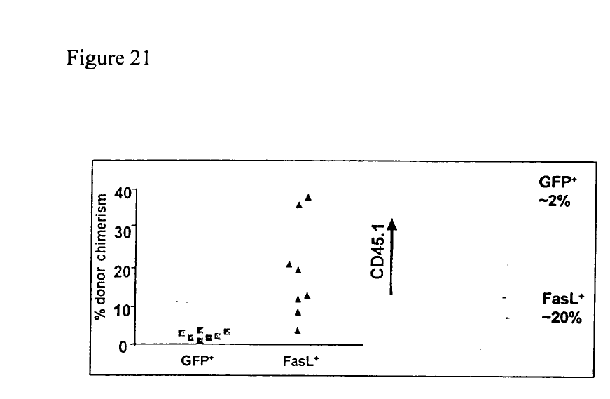

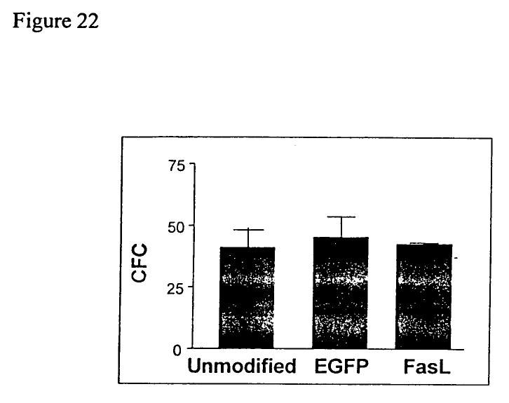

HSCs from B6.SJL mice (CD45.1⁺) were transduced with either the GFP or FasL LV prior to IV transplant into sub-lethally irradiated recipient C3H.SW (CD45.2⁺) minor-mismatched mice. Mice transplanted with FasL⁺ HSCs had significantly higher levels of donor chimerism than those that received control HSCs, both in BMs (FIG. 21) and spleens (mean donor chimerism in spleen was 15.8±3.8% for the FasL⁺ group versus 1.5±0.5% for the GFP⁺ group). 2 additional mice transplanted with untransduced HSCs had 0.5 and 1.6% donor cell engraftment in BM. BM cells from these allo-transplanted mice were assessed for CFCs. No significant differences were observed in numbers or types of CFCs from BM cells in the FasL− vs GFP-transduced groups of mice (FIG. 22).

FIG. 21 shows that mice transplanted with FasL⁺ allo HSCs had enhanced engraftment. B6.SJL HSCs were transduced with either control GFP or FasL vector (transduction efficiency=10-20%), then transplanted (10⁵ cells/mouse) into separate 400 cGy irradiated C3H.SW recipients. 3-12 weeks later, mice were sacrificed and organs analyzed for correlated expression of CD45.1 and GFP. Each point on the scatter plot (left panel) is representative of a single mouse from 1 of 3 separate experiments. The 2 right panels show examples of CD45.1 immunostaining of the median-level engrafted mouse BM from the GFP and FasL groups.

FIG. 22 shows that mice transplanted with FasL⁺ HSCs did not have diminished numbers of BM CFCs. BM from the allo-transplanted mice (FIG. 21) was assayed for CFCs. Shown are the average (±SEM) numbers of CFCs per 10⁵ plated mouse BM cells.

Mice Transplanted with FasL⁺ HSCs Did Not Have Significant Hepatic Toxicity or Immune Impairment

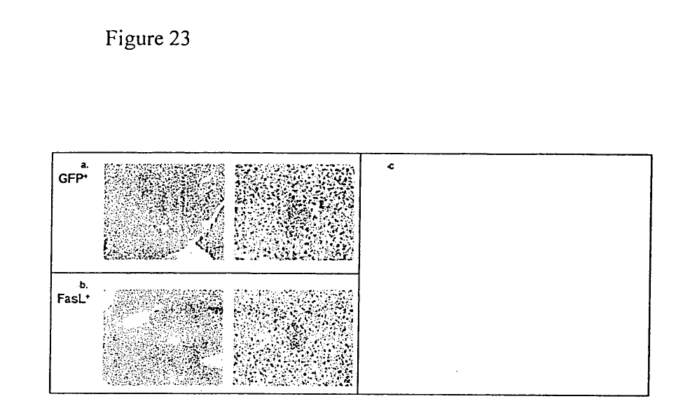

A concern with expressing FasL in HSCs is the potential for in vivo toxicity due to FasL. Mice transplanted with syngeneic or allo FasL⁺ HSCs were not notably different from control groups in overall health, or on gross pathology at autopsy. Since hepatic cells express high levels of Fas, and since hepatoxicity was reported after administration of anti-Fas Mab, we evaluated whether transplant with FasL⁺ HSCs produced histologic hepatotoxicity. Histologic analysis revealed no detectable injury to hepatic cells and no difference in the levels of inflammation in the livers of mice that had received a transplant of FasL⁺ vs GFP⁺ control HSCs (FIG. 23, A, B). To assess the immune responsiveness of allo-transplanted mice, splenocytes were taken at the time of sacrifice and used as responders in an MLR to a 3^{rd} party irradiated BALB/c splenocytes (H₂^{d}). No significant difference was observed in the levels of proliferation between the 2 groups (FIG. 23).

FIG. 23 shows that mice transplanted with FasL⁺ HSCs did not have hepatocellular injury or enhanced hepatic inflammation, and the mouse cells retained immune responsiveness to a 3^{rd} party alloAg. Hematoxylin-eosin stained slides of livers from the allo-transplanted mice (FIG. 22) were examined (Dr. F. Racke, JHMI Dept of Pathology). Representative sections from (a) a control GFP-transplanted mouse and (b) a FasL-transplanted mouse. Mild hepatic inflammation was noted in both groups (probably due to the recent BMT), but the results were not different between groups. (c) Splenocytes (2×10⁶/well) from transplanted mice were incubated as responders with an equal number of irradiated (3000 rad) allo 3^{rd} party stimulators (BALB/c spleen cells). The MLR results (mean±SEM) are from a total of 8 mice for each of the GFP or FasL groups, and 3 control C3H.SW mice, taken from 3 separate experiments.

Mice Transplanted with FasL⁺ HSCs Responded to a Listeria monocytozenes Challenge

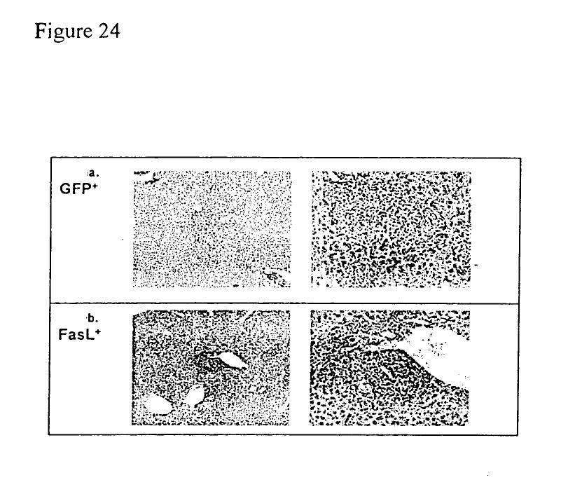

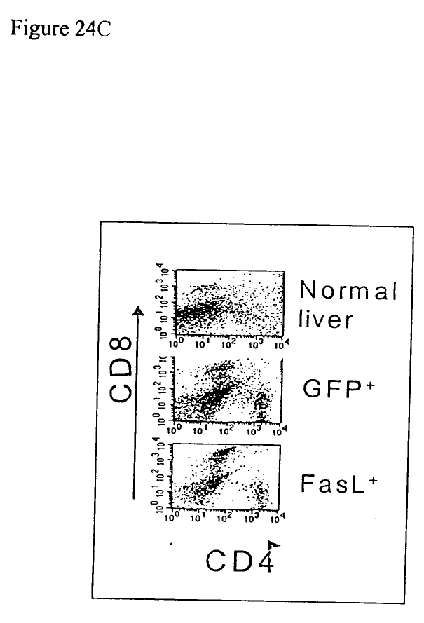

To further evaluate hepatotoxicity and to test the immune responsiveness of the transplanted mice, we challenged transplanted mice with a sublethal dose of Listeria monocytogenes as a model infectious agent. Listeria was selected as the 1^{st} infectious challenge we used, since it produces hepatic inflammation, and thus any inflammatory or hepatic in vivo toxicity of FasL⁺ HSCs or their progeny might be highlighted by the response to this challenge. In addition, since T cells die in the liver based on Fas-FasL interactions, significant FasL-mediated toxicity should prevent accumulation of T cells recruited in response to this challenge. BALB/c mice, known to be susceptible to Listeria from prelim studies, were lethally irradiated (850 cGy) and transplanted with 10⁵ syngeneic BALB/c HSCs that had been transduced with either GFP or FasL LV. 3 weeks after transplant, mice were tail bled to verify the presence of modified cells: by FACS, 14% of the GFP group and 11% of the FasL group were transduced. All mice were then injected with a sublethal dose of attenuated Listeria monocytogenes bacteria. All mice in both groups exhibited decreased activity, starting 1 day after Listeria injection. 4 days after Listeria challenge, all mice had mild inflammation in the liver, with no gross differences between the 2 groups (FIGS. 24A, B). In addition, all mice had high numbers of T cells in the livers in response to this challenge (FIG. 24C).

FIG. 24 shows that mice transplanted with syngeneic FasL⁺ HSCs responded to an antigenic infectious challenge. (a, b) Representative liver histologic sections from mice 4 days after IP injection of 10⁶ Listeria monocytogenes cfu. Significant hepatic inflammation was present in all mice. Spleens were unremarkable. (c) representative FACS plots of CD4 and CD8 cells in the liver crush of a normal uninfected mouse, and livers from a GFP-transplanted and a FasL-transplanted mouse.

Transplanted Mouse FasL⁺ HSCs Generated Transgene-Containing Progeny

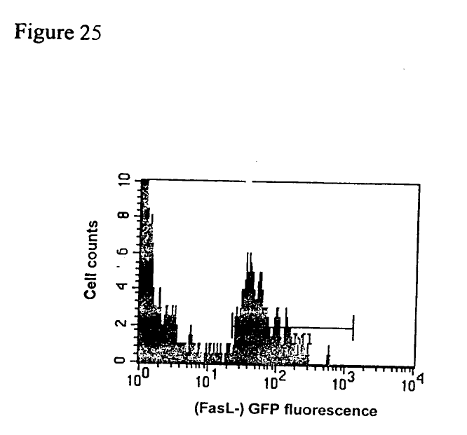

Irradiated C3H.SW mice were transplanted with FasL+B6.SJL HSCs. FIG. 25 shows that 14% of donor cells in a DC-enriched cell preparation contained the transgene. Similar numbers of donor B and T lymphocytes were GFP⁺ (not shown), and this was essentially the same as the % HSCs initially transduced (by assessment of cells cultured for 2 days post transduction).

FIG. 25 shows that transplanted FasL⁺ HSCs generated FasL⁺ DCs in vivo. C3H.SW mice were irradiated (800 cGy) prior to transplant with LV-transduced FasL⁺ B6.SJL (CD45.1⁺) HSCs. 4 weeks after transplant, mice were sacrificed, and spleens were collagenase digested to enrich for Dcs. The FACS histogram shows gated CD45.1⁺/CD11 chigh donor Dcs. 14% of donor cells in this CD-enriched fraction were FasL⁺ (based on fluorescence due to the transduced GFP-FasL fusion protein).

GVHD-Like Syndrome Xenogeneic Model

We showed that transplanted human T lymphocytes cause dose-dependent organ infiltration and a GVHD-like syndrome in NOD/SCID mice. On a per cell basis, T cells from PBSC were more potent at causing GVHD than were T cells from harvested BM. Cord blood T cells had the lowest GVHD potential. This provides an excellent in vivo model system to evaluate the effect of transduced FasL⁺ human DCs or HSCs on the function of human CTLs that can proliferate in NOD/SCID mice (See Research Design). We have recently found that transplant of very small numbers of human T cells to NOD/SCID/□2^{null} mice results in “pseudo-engraftment” (ie generation of detectable numbers of mature T cells derived from mature T cells, rather than HSCs). Thus, this should result in an even more sensitive model for the experiments testing effects on human T cells.

Mouse Ear-Heart Transplant Model

In a rodent model of in utero tolerance induction, we demonstrated multilineage hematopoietic chimerism and indefinite tolerance in recipients of allo skin, cardiac, and lung allografts. Furthermore, marked prolongation of cardiac allograft survival in a non-human primate (Rhesus) transplant model with brief induction co-stimulatory blockade in the absence of adjuvant post-transplant immunosuppression has been demonstrated. Based upon prelim work in rodent models, this combined specific induction immunotherapy was comprised of humanized anti-CD4Ig and human CTLA4-Ig. The mean graft survival time was 47 days in treated recipients, compared to <10 days in untreated controls.

### Example 4

### Assessing Whether FasL⁺ DCs Specifically Reduce an Alloimmune Response and Enhance HSC Engraftment

FasL⁺ DCs Will Delete Anti-Allo T Cells In Vitro, Reducing the Alloimmune Response

At least a large subset, if not all, activated anti-allo T/NK cells are susceptible to FasL-mediated killing (Prelim Results). We confirm and extend our initial results to conclusively determine how effectively and how specifically donor (graft) strain DCs transduced to express FasL constitutively will kill recipient (host) strain anti-donor T/NK cells, as well as to elucidate the underlying immuno-hematopoietic biology. In the first set of experimnets, we test whether mouse FasL⁺ donor strain DCs selectively kill unprimed allo T cells directed against the donor during in vitro incubation. We then determine the molecular status of the Fas and related death pathways in activation phase-defined subtypes of surviving anti-donor T cells.

Experimental Approach

In our initial results, FasL⁺ DCs killed anti-allo T cells and inhibited allostimulated T cell proliferation. We will further analyze this effect by determining the specificity of the inhibition, eg does T cell recognition of the alloAg on the DCs confer specificity in FasL-mediated killing/inhibition? Our initial results suggest that the FasL⁺ DC-mediated inhibition of the response in vitro is due at least partially to expression of alloAg by the stimulator cells, since expression of FasL by syngeneic DCs inhibited the MLR less potently. We next investigate this in greater depth by using 2C transgenic (Tg) mice. 2C Tg mice express the rearranged TCR from a cytotoxic T cell clone recognizing MHC class I L^{d}. Tg T cells expressing the 2C TCR are positively selected in the thymus by H2 K^{b} and are detected with the anti-clonotypic 1B2 Mab. We determine if transduced FasL⁺ DCs induce specific apoptosis of alloimmune CTLs by incubating naive, unprimed “responder” splenocytes from 2C mice (on a C57BL/6 (B6) background) with ex vivo generated DCs (in a 10:1 ratio of 2C to FasL⁺ CB6F1 (C57BL/6×BALB/c) DCs prepared as in Prelim Results) vs 5 types of control DCs (GFP-transduced or untransduced CB6F1; FasL- or GFP-transduced or untransduced congenic B6.SJL). Cell concentrations and 2C:DC ratios will be titrated to obtain the highest possible specificity. After 1-3 days (time course) in vitro incubation of 2C cells with FasL⁺ or control DCs, we will measure apoptosis of the treated 2C CD8⁺ T cells by FACS. We anticipate that CB6F1 FasL⁺ DCs will induce apoptosis of 2C CD8⁺ T cells, while CB6F1 FasL⁻ DCs should not. FasL⁺ B6.SJL DCs should induce no (or less) apoptosis, since they do not express the cognate L^{d} for the 2C CD8⁺ T cells to engage. In our own and other similar in vitro systems, CTLs have been specifically eliminated by FasL⁺ DCs expressing a given Ag.

In addition, we will quantitate expression of Fas, FLIP, and other pro-apoptotic and anti-apoptotic regulators (eg FasL, BCl-x_{L} and other Bcl-2 family members, survivin and other IAP family members) in 2C CD8⁺ T cells over this time course, using multiplex qPCR, FACS, and Western blotting (Prelim Results). Subsets of 2C CD8⁺ T cells at different phases of activation will be FACS-purified, using Mabs including CD25, CD45RA, CD45R0, etc. These studies will address the controversy over the relative Fas sensitivity of T cells in the early clonal expansion phase of activation, and will show whether or not subsets of alloreactive cells are sensitive to killing by FasL⁺ DCs, elucidating the cellular and molecular mechanisms in this model. Next, we will quantitate the effect of incubation with FasL⁺ DCs on the in vitro proliferative (MLR) and cytotoxic effector (CTL) functions of the 2C CD8⁺ T cells. Cell concentrations and 2C:DC ratios will be titrated, as above, to achieve the maximum selective inhibitory effect.

Next, we test the ability of the DC-treated, immunopurified 2C CD8+ T cells (1B2⁺/CD8⁺) to proliferate in vivo in response to alloAg by IV transfer to 750 cGy (near-lethally) irradiated (semi-)allo CB6F1 mice. In vivo proliferation of 2C CD8+ T cells will be quantitated by FACS in mouse blood, spleen and lymph node 3-21 days (twice weekly for blood, weekly for organs) after IV infusion of 6−10×10⁶ DC-treated 2C T cells vs untreated or control-treated (5 types of control) 2C CD8⁺ T cells. Adoptive transfers and in vivo identification of 2C CD8⁺ T cells will be done as in Prelim Results and in the prior descriptions of similar experiments using this model in which 2C CD8⁺ T cells have been detected for >2 months after infusion. At the (bi-)weekly timepoints, 2C CD8⁺ T cells will be immunopurified from mouse blood/organs by FACS and assessed for expression of apoptotic regulators and activation markers, in order to molecularly describe the apoptotic susceptibilities of cell types that survive treatment with FasL⁺ DCs and proliferate in vivo in response to alloAg. This is especially interesting, since in Tg mice expressing FasL via a T cell-specific promoter, substantial numbers of T cells were present and resistant to Fas-mediated apoptosis by an unknown mechanism. Each datapoint will contain 6 strain/age/sex-identical mice. We expect that CB6F1 DCs will present their H2L^{d} to 2C CD8⁺ T cells. If a CB6F1 DC subset is FasL⁺ (expressed constitutively via transduction), those FasL⁺ DCs might kill (some or all of) the 2C CD8⁺ T cells that interact with them during sensitization, even though T cells are not thought to be highly sensitive to FasL-mediated killing prior to activation. In contrast, control FasL⁻ (ie untransduced or GFP-transduced CB6F1) DCs should activate the 2C CD8⁺ T cells. FasL⁺ syngeneic DCs should induce no (or less) apoptosis, since L^{d} alloAg should be required for the DCs to engage 2C CD8⁺ T cells. In our own initial results and similar in vitro systems, Ag-specific T cells have been selectively eliminated by FasL⁺ APCs. The effects on the in vitro MLRs and in vivo proliferative responses of the treated 2C CD8⁺ T cells should provide similar results, indicating a reduction of 2C CD8⁺ T cells by the FasL⁺ CB6F1 DCs, in contrast to the control DCs.

We then assess the effect on anti-allo T cells of administration of FasL⁺ CB6F1 DCs (or the above 5 controls) to mice. 2C mice will be injected with titered doses of DCs. We then bleed mice bi-weekly for 3 weeks to quantitate 2C CD8⁺ T cells by FACS. After 3 weeks, we will sacrifice the mice, and assess splenocytes, thymocytes, etc. In the initial results reported above, CTLs have been specifically eliminated by FasL+CB6F1 DCs. However, the response in 2C mice may well be too strong (ie too many alloreactive T cells) to diminish detectably in our system. If so, we will adoptively transfer 2C cells to B6.SJL mice and generate mice with only 0.5-5% 2C CD8⁺ T cells. One of these models will enable us to quickly determine the numbers of CB6F1 FasL+ DCs necessary for maximal killing of 2C CD8⁺ T cells, as well as the effect of time of DC administration in relation to alloAg priming. Subsets of surviving 2C CD8⁺ T cells at different phases of activation will be FACS-purified from the mice, and assessed for levels of expression of Fas, FLIP, and other pro-apoptotic and anti-apoptotic regulators, as above. To confirm the results in a more physiological system, we will test the effect of CB6F1 FasL⁺ DCs on T cells of B6.SJL mice. We will also measure the (anticipated) decrease in allospecific MLR and CTL assays in this model system. These experiments will then be repeated using CTLs from lpr mice (on a B6 background)—since lpr mice do not express Fas, no effect of FasL⁺ DCs should be observed.

Accordingly, our first set of experiments are designed to test the efficacy and specificity of reducing alloimmune responses by incubating responder cells in vitro with allo FasL⁺ DCs. To measure any nonspecifically immunosuppressive effects of the FasL⁺ DCs in this in vitro context, we evaluate the in vitro and in vivo proliferative response of the ex vivo treated T cells to a 3^{rd} party (eg H2^{k}) control. Our initial results show that spleen cells from mice that had been transplanted with FasL⁺ allo cells responded in vitro to a 3^{rd} party stimulator. Nevertheless, it is possible that the proposed experiments may show that the in vitro treatment with FasL⁺ cells is highly nonspecifically immunosuppressive. We might be able to increase the specificity by reducing the cell concentrations and finding the most effective ratios of allo T cells:DCs. In addition, sFasL might contribute to nonspecificity; this could be addressed by transducing the non-cleavable delFasL cDNA. Finally, the specificity of FasL⁺ cells might be much greater in an in vivo setting, where the TCR-specific binding of alloimmune anti-donor T cells selectively to the allo DCs (including those transduced to be FasL⁺) might be a larger factor than in vitro. Therefore, even if the in vitro treatment with FasL⁺ DCs in the first set of experiments is found to result in nonspecific immunosuppression, in vivo treatment with FasL⁺ DCs will still be investigated.

Vectors and Delivery

In the above experiments, we utilize lentiviral vectors (LV) to transduce FasL, since we have shown that LV can efficiently transduce mouse and human DCs and HSCs. However, stable transduction of DCs or HSCs may not be necessary experimentally or desirable for future translation of this FasL⁺ DC strategy to clinical trials. Therefore, we evaluate the comparative efficacy of other delivery systems, such as transiently transfected (eg electroporation) or classic oncoretroviral vector (RV) transduced DCs to mediate the deletion of alloimmune cells.

We further investigate allo-tolerizing by administration of CTLA4-Ig or anti-CD40L. We compare these approaches quantitatively for potency and specificity. Since the 2 methods (FasL killing and co-stimulatory blockade) work by different mechanisms, we investigate whether their effects are additive or synergistic in vitro. Possible additional effects by treatment with soluble TNF or DR-TRAIL may be suggested by our molecular analysis of apoptotic pathways in the T cells that survive treatment with FasL⁺ cells. Interesting recent evidence suggests that rapamicin may be additive or synergistic. Most of the experiments in this proposal focus on T cells, since it is established that activated T cells are the principal mediators of GVHD. Nevertheless, NK cells may also contribute to GVHD. Activated NK cells express Fas, and are anticipated to be sensitive to the same maneuvers involving engineered FasL⁺ cells that kill activated T cells.

Administration of FasL⁺ DCs In Vivo Enhances Engraftment of Transplanted Allo HSCs

In Aim the next set of experiments, we test whether transduced FasL⁺ donor strain DCs function in vivo to (a) selectively kill mouse host T cells directed against the donor, (b) selectively reduce the host mouse anti-donor alloimmune response, (c) reduce in vivo graft rejection in mouse transplant models, and (d) selectively kill human T/NK cells. As in the first set of experiments, we quantitate death pathway regulatory molecules in T cell subsets in the context of these experiments, to gain insight into the underlying mechanisms. In vivo administration of FasL⁺ cells may result in nonspecific immunosuppression or organ toxicity. Accordingly, we carefully examine mice for immune function and organ toxicity. Further, we propose investigating technologies to delete FasL⁺ cells (or inhibit their expression of FasL), once the desired alloAg-specific tolerance has been achieved.

Experimental Design and Anticipated Results

Our initial results indicated that allo engraftment of B6.SJL donor cells in C3H.SW hosts was modestly enhanced if the recipients were pre-treated with FasL⁺ B6.SJL DCs. We extend these studies to determine whether we can further increase allo donor cell engraftment and determine the mechanism of any FasL-resistant HVG response. In order to determine how specific the decrease in response is for alloAg, we start by repeating the initial studies using the CB6F1 into 2C model, where we can follow the allo response sensitively and specifically.

First, we assess the effects on 2C CD8⁺ T cells of administration of FasL⁺ CB6F1 DCs (or the above 5 controls) to mice containing 2C CD8⁺ T cells. Titered repeated doses of DCs (Prelim Results) are administered to naive or allosensitized 2C mice (or B6.SJL mice with adoptively transferred 2C CD8⁺ T cells, as above). We then bleed the mice twice weekly for 3 weeks to quantitate 2C CD8⁺ T cells by FACS. After 1-3 weeks, we sacrifice the mice, and assess 2C CD8⁺ T cells from spleen, thymus, etc, including by MLR and CTL assays. In our initial results, CTLs have been specifically eliminated by FasL⁺ APCs. This 2C model will enable us to quickly determine the numbers of CB6F1 FasL⁺ DCs necessary for optimized killing of alloimmune T cells, as well as the effect of time of DC administration in relation to alloAg priming. To confirm these results in a more physiological system, we test the effect of FasL⁺ CB6F1 DCs on T cells of B6.SJL mice (and lpr mice, as a control). We then measure the (anticipated accompanying) decrease in allospecific MLR and CTL assays.

Next, we develop a model for allo BMT of sublethally irradiated 2C recipient mice (or B6.SJL mice with adoptively transferred 2C CD8⁺ T cells, as above) with CB6F1 “HSCs” (operationally defined as “Lin-” marrow cells enriched using Stem Cell Technologies immunomagnetic kit). We first titrate (from 1−20×10⁵ HSCs) to determine the lowest number of CB6F1 HSCs necessary to engraft (ie generate mixed hematopoietic chimerism with 10-50% donor cells) in sublethally irradiated 2C mice (or B6.SJL mice with adoptively transferred 2C CD8+ T cells, as above), titering the radiation dose down from 700 cGy. Mice bled at intervals, then sacrificed (monthly) at 1-6 months post-transplant, and engraftment of CB6F1 HSCs determined by (a) flow cytometric quantitation of the number of H2^{d+}/CD45.2⁺ cells in each of multiple lineages (ie, myeloid, B, T, NK, DC, etc), and (b) donor CFCs (by in situ immunostaining for H2^{d} and CD45.2). This model allows us to quantify the effect of graded doses of FasL⁺ DCs on the levels of human lymphohematopoietic cells, at fixed doses of HSCs and irradiation. We then investigate whether treatment of mice with FasL⁺ DCs (vs control DCs) facilitates the same levels of engraftment with lower doses of HSCs and/or irradiation. Once we have established this allo BMT model, we transplant CB6F1 HSCs into sublethally irradiated 2C mice (or B6.SJL mice with adoptively transferred 2C CD8⁺ T cells, as above), after recipient treatment with a series of injections of the 6 types of DCs described above. We anticipate that pre-treatment with FasL⁺ CB6F1 DCs will increase the levels of donor cell engraftment at a given HSC dose, decrease the numbers of HSCs needed to generate a given level of donor cell engraftment, and decrease the radiation dose required to attain a given level of donor cell engraftment. As in the first set of experimnets, we will immunopurify the 2C CD8⁺ T cells from mice at time points during the experiments and determine their expression of Fas pathway and other apopotic pathway molecules, to elucidate the mechanisms of resistance to FasL in surviving 2C CD8⁺ T cells.

Next, we use this model to evaluate the effects of co-transplant of 10⁵-10⁷ FasL⁺ CB6F1 DCs vs control DCs and the above determined dose of CB6F1 HSCs. We determine whether administration of FasL⁺ DCs with the HSCs results in higher level donor engraftment, as above. We compare whether administering FasL⁺ CB6F1 DCs prior to CB6F1 HSCs allows for higher level donor engraftment (at lower HSC dose and/or with lower radiation doses) than simultaneous co-transplant of FasL⁺ CB6F1 DCs. We then also investigate the effects of combining prior plus simultaneous co-transplant of FasL⁺ CB6F1 DCs.

Next, we test the effects of FasL⁺ CB6F1 DCs on non-Tg B6.SJL splenic T cells from recipients. Quantitation of death pathway molecules and T cell activation markers are performed as outlined above. Our initial results, using different mouse strains, demonstrate that engraftment is enhanced by FasL⁺ donor-strain DCs,. Next, we enhance this effect by using larger numbers of injections with greater numbers of FasL⁺ DCs that are more highly transduced (multiply transduced and FACS-sorted after transduction). In addition, we investigate combinations of this FasL⁺ DC pretreatment regimen with potentially additive or synergistic immunosuppressive manuevers, eg co-stimulatory blockade or rapamicin.

Potential lympho-hematopoietic and organ toxicity of FasL⁺ DCs is carefully assessed by blood cell counts and histology in transplanted mice, since FasL⁺ DCs or their progeny may interfere with immune function or be hepatotoxic. We evaluate the in vitro and in vivo proliferative response of the in vivo treated T cells to a 3^{rd} party (H2^{k}) control. Our initial results show that spleen cells from mice that had been transplanted with FasL⁺ allo HSCs responded in vitro to a 3^{rd} party stimulator. CTL assays are conducted on transplant recipient mice (B6.SJL: H2^{b}) immunized post-transplant with H2^{k} splenocytes. We next evaluate the ability of mice treated in vivo with FasL⁺ DCs to respond to Listeria monocytogenes challenge, which normal mice control with mobilization of T cells to the liver (see initial results above). We then employ other types of Ags used routinely by our Cancer Center colleagues, eg Vaccinia-HA and CMV (BMT-relevant challenges), and assess humoral and cellular limbs of the immune response. In addition, we autopsy a representative sample of the mice exhibiting allotolerance, with special attention to the cytology of livers, the potential presence of inflammatory cell infiltrates, and the immunohistochemistry of lymphoid tissues. Transduced FasL⁺ hematopoietic cells may cause damage to mouse organ-systems, although we have not observed this in experiments to date which have been carried out to 12 weeks post transplant. If toxicity is observed, we will test the use of the delFasL mutant (Prelim Results), which should eliminate sFasL and might thereby reduce the nonspecific toxic effects.

It is not known where in the allo host these in vitro generated FasL⁺ DCs will migrate or how long they will persist. Accordingly, transplanted mouse organs are microscopically screened for the presence of GFP⁺ cells. The presence of allo FasL⁺ DCs in the thymuses of 2C recipients, along with diminished thymic cellularity and inhibition of the alloimmune response, would be consistent with in vivo action at the sensitization stage. If there is organ toxicity and if DCs persist long after tolerance is established, we would test eliminating the FasL⁺ cells at monthly timepoints after donor chimerism is achieved, eg by a Herpes simplex thymidine kinase/ganciclovir or similar approach. Use of a conditional FasL-fusion protein approach is an alternative.

Finally, we repeat these experiments using immunopurified normal human donor blood T cells as responder cells, and allo FasL⁺ (vs control) human BM- or blood-derived DCs as the mediators of FasL induction of alloimmune T cell death. We begin by using allo FasL⁺ (vs control) DCs generated from CD34⁺ cells. Responders and DCs are co-incubated for 1-5 days (time course) over a range of cell concentrations and ratios, as suggested by the results of the above mouse cell experiments. Quantitation of apoptosis, in vitro proliferative capacity and death pathway regulators is performed for the human T cells, and the results are determined for immunopurified activation-phase-subsets of human T cells, as above for the mouse T cells. Our initial results show that in vitro treatment of human HSCs with sFasL does not impair their ability to form CFCs or generate multilineage engraftment in NOD/SCID mice. Utilizing conditions which reduce the alloimmune effectors significantly, we incubate human BM (or cord blood or mobilized blood) cells (containing “contaminating” aspirated blood T cells, or with additional admixed blood T cells) with allogenic FasL⁺ (vs control) DCs, and then determine if CFC and NOD/SCID engraftment capacities are affected by ex vivo exposure to allogenic FasL⁺ (vs control) DCs and this alloimmune response.

### Example 5

### Evaluating Tolerance to Cardiac Allografts in Hematopoietic Chimeras Generated Using FasL⁺ DCs/HSCs

Lympho-hematopoietic macrochimerism has been demonstrated to lead to long-term donor-specific tolerance in several animal model. In the next set of experiments, we achieve chimeric allo tolerance to cardiac allografts using prior BMT facilitated by FasL⁺ donor DCs/HSCs. We demonstrate whether this approach effectively induces tolerance to solid organ (ie cardiac) allografts alone or in combination with a co-stimulatory blockade. Furthermore, we determine what levels of chimerism are required to achieve durable tolerance. The experiments are based upon a mouse model to determine feasibility and to obtain an optimized protocol for future application in a non-human primate model of heterotopic cardiac transplantation.

Hematopoietic Macrochimeras Established Via AlloBMT with FasL⁺ DCs/HSCs are Mmunologically Tolerant to Heterotopic Cardiac Allografts

We utilize allo heterotopic cardiac transplantation (mouse ear-heart model) to challenge mice that are lympho-hematopoietic macrochimeras, produced based on the results of BMT with FasL⁺ DCs/HSCs. Our initial results suggest that apoptosis of alloreactive T cells and hematopoietic chimerism can be induced using non-ablative BMT conditioning plus FasL⁺ donor Dcs/HSCs. B6.SJL cardiac allografts are prepared based upon the published mouse ear-heart methodology (see e.g. Fulmer et al. (1963) 113: 273-6). Each adult 2C or B6.SJL recipient mouse (tolerized by FasL⁺ CB6F1 DCs/HSCs in the context of CB6F1 into B6.SJL BMT, confirmed by presence of macrochimerism, vs control, as in Aims 1-2) will be transplanted with a CB6F1 neonatal donor heart placed into a subcutaneous pocket created on the ear. Cardiac allograft viability will be confirmed by visual inspection of graft contractions under low power magnification (10-20×), as confirmed by electrocardiography. All allografts display contractile function by postoperative day 6; untreated allo recipients uniformly display graft rejection (absence of contractions) by day 8. The Mann-Whitney U test will be used to determine statistically significant (p<0.05) prolongation of cardiac allograft survival.

Co-Stimulatory Blockade Augments the Tolerogenic Capacity of FasL⁺ Hematopoietic Cells for Both Multiple Minor and Major MHC-Mismatched Cardiac Allograft Transplants

Graft recipients are treated with a hamster Mab against mouse CD154 (MR1 hybridoma; 0.5 mg injected IP on the day of BMT) and/or mouse CTLA4-Ig (0.5 mg injected IP on postoperative day 2). The MR1 hybridoma are provided by R. J. Noelle (Dartmouth Medical School, Lebanon, N.H.), and the CTLA4-Ig molecule are provided by Bristol-Myers-Squibb. The experiments and interpretations are as explained above. We anticipate that co-stimulatory blockade will add to the tolerizing effect without detectable toxicity, but will evaluate the transplanted mice.

A significant complication with the experiments described above would be the inability to achieve indefinite cardiac allograft survival with FasL⁺ DC/HSC bone marrow transplantation with or without concomitant costimulatory blockade. Should this occur, we would first determine (1) whether macrochimerism was achieved with the initial transplantation, and (2) whether the levels of chimerism deteriorate over time if not augmented with subsequent FasL⁺ DC/HSC transfusions and/or anti-CD40L/CTLA4-Ig treatments. Alternatively, other experimental strategies would include (1) increasing the dosages of FasL⁺ DC/HSCs and/or anti-CD40L/CTLA4-Ig at the time of initial transplantation, (2) incorporating a low-dose myeloablative regimen (eg XRT) at the initial transplantation to facilitate FasL⁺ DC/HSC engraftment, and (3) utilizing a series of “inductive” FasL⁺ DC/HSC transfusions and/or anti-CD40L/CTLA4-Ig treatments.

### Example 6

### Fas/FasL-Mediated Apoptosis in GVHD and Aplastic Anemia

In the next set of experiment, we provide studies to test the concept and elucidate the principal mechanisms by which transduced DCs or HSCs constitutively expressing Fas ligand (FasL) may reduce the T (and NK) cells that mediate GVHD without overwhelming organ toxicity or general immunologic impairment. The first such experiment attempts to accelerate and enhance the natural homeostatic role of FasL during the course of the alloimmune response, with the potential outcome that alloimmune T cells may be killed earlier in their activation/expansion, or killed more potently later during activation-induced cell death (AICD), by transduced FasL⁺ DCs or HSCs/progeny, respectively. As we investigate this, we will examine the apoptotic pathways in alloimmune cells. We tentatively plan a clinical trial to reduce GVHD from allo donor leukocyte infusions (DLI) by ex vivo incubation of DLI cells with transduced FasL⁺ host DCs.

The results of the first set of experiments will also provide modeling information on the application of transduced FasL⁺ cells to reduce immune attack in severe aplastic anemia SAA (Second Set of Experiments). We expect that transduced FasL⁺ HSCs might potentially be toxic, especially in vivo. Therefore, we will in parallel investigate technologies to limit potential FasL toxicity, e.g. by transducing only a small percent of high quality HSCs, by employing a FasL deletion mutant that cannot be cleaved to release soluble FasL (sFasL), by eliminating the transduced cells (or their FasL expression) after tolerance to HSCs has been established, or by using lineage/stage-specific promoters to restrict FasL expression. A novel FasL⁺ cell therapy approach to reduce effector lymphocytes attacking host cells in GVHD (first set) and severe aplastic anemia SAA (second set) may eventually be used, ex vivo or in vivo in transplants for SAA, PNH and other diseases, possibly in conjunction with other immunosuppressive methodologies.

Engineering FasL⁺ Host DCs or HSCs to Kill the Cellular Effectors of GVHD

In this set of experiments, we will used ex vivo incubation with FasL⁺ recipient strain DCs or HSCs to kill alloreactive donor (graft) T cells and thereby selectively reduce the GVH response. Furthermore, we use in vivo administered FasL⁺ host strain DCs or HSCs to generate tolerance for host cells in allo transplant models. Potential toxicity of in vivo transplanted FasL⁺ HSCs can be limited by further genetic engineering.

In order to determine the lowest effective levels of cellular FasL expression and the lowest numbers of FasL⁺ cells that are required to generate tolerance, we investigate deleting the FasL⁺ cells after tolerance is observed, since permanent immunosuppression is not necessary to prevent/treat clinical GVHD. For example, if we find that a short period of FasL expression in cells is highly efficient at reducing GVHD, transiently transfected DCs or HSCs might be optimal for clinical trials. Specifically, transiently transfected FasL⁺ human DCs (eg transfected using electroporation) might be fully sufficient for ex vivo treatment of donor grafts. If a somewhat longer duration expression of FasL is necessary, an oncoretroviral vector (RV) might be considered for clinical trials. Only if very long-term FasL expression is necessary will we need to engineer around toxicity potentially associated with longer duration of large numbers of transduced FasL⁺ HSCs in the host, using LV vectors in clinical trials.

GVHD is the Major Limit on Use of BMT

We first stably transduce lympho-hematopoietic stem-progenitor cells (HSCs) with genes that are then expressed in vivo for at least several months. This ex vivo viral vector transduction technology allows us to begin to engineer HSCs and their progeny (focusing on DCs, which can also be transduced efficiently as they develop from HSCs ex vivo in an attempt to develop improved cellular tools to treat disease and reduce complications in BMT.

While allo BMT is an important treatment option in several malignant and nonmalignant disorders, including severe aplastic anemia (SAA) and paroxysmal nocturnal haemoglobinuria (PNH), GVHD continues to be the major overall factor limiting use of BMT, particularly for nonmalignant disorders where graft vs tumor effects play no role. One way to avoid GVHD is to utilize autologous HSCs as the transplant graft. However, not all patients may have sufficient, cancer-free autologous HSCs to generate prompt, sustained engraftment, even after ex vivo manipulations. Thus, there will still be patients for whom allo BMT would be preferred to autologous BMT, in SAA and other diseases. Some patients have an HLA-matched sibling donor to provide HSCs, but most need to utilize grafts from alternative donor BM, mobilized blood (PBSC), or cord blood (CB). Even with CB, GVHD is a serious problem in alternative donor BMT.

Cellular Effectors of GVHD

GVHD is mediated principally by T lymphocytes. In mouse BMT models, mature CD4⁺ T cells are the major mediators of GVHD in some BMT strain combinations, and CD8⁺ T cells in others. Probably, both CD4⁺ and CD8⁺ activated T cells play roles in human GVHD. NK cells play a role in rejection of experimental allografts, and have also been implicated in GVHD

Current regimens for GVHD prevention/treatment are not fully effective, may be organ-toxic, and are nonspecifically immunosuppressive: Ideally, tolerance would be induced by a method that specifically deletes alloreactive effector cells from the graft. Rigorous T cell depletion or isolation of transplanted HSCs will remove mature T cells and thereby reduce GVHD. However, in the absence of donor T cells, graft rejection becomes a major clinical problem in all uses of BMT, as does reduction of graft vs tumor immunologic effects in BMT for cancers. Incomplete T cell depletion, or depletion of only a subset of the T cells, results generally in moderately efficacious GVHD prevention. Current GVHD treatment/prevention regimens include small molecule drug regimens, and anti-thymocyte globulin (ATG) and several monoclonal antibodies which have been used in combination with drug regimens. Nevertheless, many cases of GVHD are resistant to all of these regimens, and all of these non-specific immunosuppressive treatments increase the patient's risk of opportunistic infection. In addition, immuno-incompetent cancer patients may not respond against endogenous tumor or to tumor vaccines early after transplant, when the patient has minimal residual disease. Therefore, other ways to prevent/treat GVHD while minimizing general immunosuppression is imperative for effective application of BMT, not only for the present, but also for anticipated future new anti-infectious and anti-tumor strategies that depend on functional immunity. For example, recently, an innovative attempt to specifically tolerize to host antigens (Ags), by incubation of the donor BM with irradiated host cells in the presence of CTLA4-Ig, resulted in a low rate of GVHD in a pilot clinical trial. The use of co-stimulatory blockade (with CTLA4-Ig or anti-CD40L Mab) has also been shown to reduce GVHD in preclinical models, but the reduction of GVHD has mostly been partial, not complete.

Accordingly, we provide a biologically-based tolerizing strategy, using FasL⁺ DCs or HSCs, that might be used as an alternative to, or in combination with co-stimulatory blockade or other selective immunosuppressive methodologies, potentially without (or with reduced doses of) current pharmacologic immunosuppressive and conditioning regimens. This approach will exploit the Fas pathway in treating GVHD.

The Fas/FasL pathway is particularly important in immune response regulation. At the start of a T cell immune response, Ag-specific T cell clones undergo IL2-dependent clonal expansion and generate large numbers of effector cells in response to antigenic challenge via DCs and other Ag-presenting cells (APCs) displaying the necessary co-stimulatory molecules and the given peptide Ag on the appropriate major histocompatibility complex (MHC) molecule. Sensitizing DCs may express Fas and/or FasL, but it appears that their expression of FLIP makes them resistant to FasL-mediated apoptosis. While some studies indicate that responding T cells are quite resistant to apoptosis at the time of their interaction with APCs and that co-stimulation increases T cell expression of FLIP and BCl-x_{L}, other studies (including our results) indicate that artificially FasL⁺ APCs can down-modulate immune responses. Accordingly, in this set of experiments we confirm and extend our initial results indicating that transduced FasL⁺ DCs can inhibit generation of an alloimmune response by action at this early phase of T cell activation.

The magnitude of a specific immune response is down-modulated by activation-induced cell death (AICD) of activated T lymphocytes, and the FasL/Fas pathway is the principal mediator of AICD. Activation of T cells increases their FasL expression as well as their sensitivity to FasL-mediated apoptosis. These activated T cells specifically reduce the size of the activated T cell clones by FasL-mediated suicide and fratricide; notably, this physiologic AICD may involve large subsets of the individual's T cells, yet does not cause general damage to other Fas-expressing organs/tissues. It is not clear why this killing is not 100% effective, but small numbers of activated T cells must survive to become memory T cells. Fas and FasL similarly mediate AICD of activated NK cells. Thus, sensitivity to FasL-induced apoptosis may represent a physiologically important vulnerability of activated donor T/NK cells. Accordingly, enhancing AICD via the Fas pathway may be a selective strategy to down-regulate a specific cellular immune response; eg organ allograft rejection can be suppressed in mice by FasL⁺ APCs. Accordingly, we utilize transduced FasL⁺ HSCs to inhibit an alloimmune response by accelerating decay of the numbers of activated alloimmune T cells during the down phase of T cell activation, and/or by action at the earlier clonal expansion phase.

Approach to GVHD Prevention/Treatment

Expression of FasL in the BMT recipient reduces intestinal and thymic GVHD. Accordingly, we utilize two genetic approaches to selectively reduce the alloreactivity of grafts. In the first approach, host strain DCs, the most potent APCs, are transduced to express FasL. These FasL⁺ host DCs have an increased ability to kill alloreactive T cells engaged at the start of an immune response. Studies have demonstrated the capacity of FasL⁺ DCs to selectively decrease alloreactivity. To investigate longer-acting FasL⁺ cells to kill alloAg-specific T cells during either or both the expansion/effector and the down phases of the immune response, we utilize a second approach: to transduce host HSCs so that they and their progeny express FasL. If FasL⁺ HSCs serve as “armed” target cells, they might even kill CTLs generated from memory cells. Thus, both the DC and the HSC approaches are attractive approaches, as they have the potential to attack alloimmune T cells at discrete phases of activation. Accordingly, we determine the contribution of targeting each phase of T cell activation in the “counterattack” via FasL, and determine which cell types (eg, DCs, HSCs or a different progeny cell type) optimally mediate each part of the counterattack.

FasL⁺ host DCs or HSCs will function ex vivo or in vivo to increase the killing of activated anti-host T/NK cells, thus decreasing anti-host immunity and reducing or eliminating the need for nonspecific pharmacologic immunosuppressive prevention/treatment of GVHD in allo BMT. The effects may be accelerated/enhanced using the natural homeostatic role of FasL during the course of the immune response, with the potential outcome that alloimmune T cells may tend to be killed earlier in their activation/expansion, or more potently later during AICD, by transduced FasL⁺ DCs or HSCs/progeny, respectively. In addition, we investigate the toxicity of these FasL⁺ cell therapy approaches and modify the procedures to minimize nonspecific toxicity. Some of the tools (eg lentivirally [LV] transduced recipient HSCs constitutively expressing FasL) are used to carry out these studies in laboratory models may well not be the actual means we use to translate our preclinical results to clinical trials. The studies, and investigation of mechanism and toxicity, and further engineering of the approaches in preclinical models, will not only lay the foundation for clinical application of FasL⁺ cells against GVHD, but will also provide increased basic understanding of the exact role of the Fas/FasL pathway in the alloimmune response.

Involvement of the Fas Pathway May in the Pathogenesis of SAA/PNH

Although most CD34⁺ cells from normal individuals lack Fas expression, some CD34⁺ cells from patients with SAA express Fas and are sensitive to Fas-mediated killing. Recently, it was found that the normal (ie the PIGA⁺ but not the PIGA-) cells in the CD34⁺ cell population from PNH patients similarly express Fas and are killed by Fas agonists. This may provide at least part of the pathophysiologic basis for (a) SAA and (b) the outgrowth of the PNH clone in the SAA patient. Accordingly, immunologic attack by CTLs against autologous HSCs is a major mechanism in SAA. Since CTLs kill target cells via Fas, as well as via perforin/granzyme pathways, we can determine the sensitivity of SAA/PNH patient CD34⁺ cells to FasL-mediated cell death and the relevant mechanisms involved.

Transduced FasL⁺ recipient DCs or HSCs are used to reduce alloimmune responses and in vivo GVHD, in selected mouse BMT models and in human-immunodeficient mouse BMT models. Furthermore, we exploit a Fas pathway sensitivity and the ability of transduced FasL⁺ autologous human HSCs to kill autoimmune (anti-HSC) CTLs from patients with SAA. These methods have applications beyond the possible development of novel biologically-based treatments for GVHD and SAA—they may be applied to controlling apoptotic pathways in the life and death interactions of immune system with hematopoietic tumors and transplanted organs/stem cells.

Mouse Model for GVHD

To test the preliminary feasibility of the standard B6 parent into CB6F1 recipient GVHD model, overnight-cultured 2C spleen cells or mixtures of B6.SJL spleen cells (as APCs) plus 2C cells (or control cell preparations; not shown) were injected IV into 450 cGy irradiated CB6F1 mice. At 6-50 days after cell transfer, transplanted mice were euthanized and splenocytes were assessed for content of CD8⁺ 2C T cells (CD8⁺/1B2⁺). Tables 1-2 show that naive or allosensitized CD8⁺2C T cells were detectable in vivo for at least several weeks after transfer to irradiated CB6F1 mice. Transplanted mouse FasL⁺ HSCs generated transgene-containing progeny. Irradiated C3H.SW mice were transplanted with FasL⁺ B6.SJL HSCs. 14% of donor cells in a DC-enriched cell preparation contained the transgene. Similar numbers of donor B and T lymphocytes were GFP⁺ (not shown), and this was essentially the same as the % HSCs initially transduced (by assessment of cells cultured for 2 days post transduction).

GVHD-Like Syndrome Xenogeneic Model

Transplanted human T lymphocytes cause dose-dependent organ infiltration and a GVHD-like syndrome in NOD/SCID mice. On a per cell basis, T cells from PBSC were more potent at causing GVHD than were T cells from harvested BM. Cord blood T cells had the lowest GVHD potential. This provides an excellent in vivo model system to evaluate the effect of transduced FasL⁺ NOD/SCID mouse DCs or HSCs on the function of human CTLs that mediate this GVHD-like syndrome in NOD/SCID mice. We have recently found that transplant of very small numbers of human T cells to NOD/SCID/□2M^{null} mice results in “pseudo-engraftment” (ie generation of detectable numbers of mature T cells derived from mature T cells, rather than HSCs). Thus, this should result in an even more sensitive model for the experiments with human cells.

Engineering FasL⁺ Host DCs or HSCs to Kill the Cellular Effectors of GVHD

At least a large subset, if not all, activated anti-allo T/NK cells are susceptible to FasL-mediated killing. Neither FasL⁺ DCs nor FasL⁺ HSCs caused major organ toxicity or immune paralysis after in vivo administration to mice. Perhaps as occurs with activated T cells which express FasL in the normal course of AICD or in mice with Tg T cell expression of FasL, significant numbers of FasL⁺ immune cells are not organ-toxic (or generally immunoablative) to the individual, in contrast to the hepatotoxic effect of a systemically administered Fas agonist.

Ex vivo incubation with FasL⁺ recipient strain DCs or HSCs to kill alloreactive donor (graft) T cells and thereby selectively reduce the GVH response.

Our initial results demonstrate that FasL⁺ DCs killed anti-allo T cells and inhibited allostimulated T cell proliferation. FasL⁺ DC-mediated inhibition of the response in vitro is due at least partially to expression of alloAg by the stimulator cells, since expression of FasL by syngeneic DCs inhibited the MLR less potently. Accordingly, we first use simple in vitro incubation of allo mouse donor cells with FasL⁺ host strain DCs (optimally) or HSCs (a) selectively kills allo T cells directed against the host, (b) selectively reduces the donor GVH immune response, and (c) reduces in vivo GVHD in BMT models. The general approach of inducing tolerance during an ex vivo incubation has clinical precedent, in that incubation of an allograft with donor cells in the presence of CTLA4-Ig induced anergy to donor alloAgs and a low incidence/severity of GVHD in patients. Although an a priori advantage of this ex vivo strategy is that it would not require in vivo administration of proliferative FasL⁺ cells, the toxicity to organs and immune function of transplanted mice will be determined. Next, the effect of this approach on human T cells in laboratory models is determined. Finally, potential clinical model trials would be used to evaluate the clinical feasibility of this ex vivo DC approach (years 4-5).

In Vivo Administered FasL⁺ Host Strain DCs or HSCs to Generate Tolerance for Host Cells in Allo Transplant Models and Use of Genetic Engineering to Limit Potential Toxicity of In Vivo Transplanted FasL⁺ HSCs

In these experiments, transduced FasL⁺ host strain HSCs or DCs function are examined in vivo for their ability to (a) selectively kill alloimmune donor T cells directed against the host, (b) selectively reduce the donor GVH immune response, (c) reduce in vivo GVHD in mouse transplant models, and (d) selectively kill human T(/NK) cells. Death pathway regulatory molecules in T cell subsets in the context of these experiments are examined to determine the underlying mechanisms. In vivo administration of FasL⁺ cells will result in more profound and long-lasting reduction of GVHD effector cells, since the presence of FasL⁺ recipient cells in vivo provides a “surveillance” of the reconstituting donor immune system that generates and maintain tolerance. However, in vivo administration of FasL⁺ cells may also result in nonspecific immunosuppression or organ toxicity, and, accordingly we utilize genetic engineering strategies (a) to restrict FasL expression to HSCs and DCs and (b) to delete FasL⁺ cells (or inhibit their expression of FasL), once the desired alloAg-specific tolerance has been achieved.

Equivalents

Those skilled in the art will recognize, or be able to ascertain using no more than routine experimentation, numerous equivalents to the specific polypeptides, nucleic acids, methods, assays and reagents described herein. Such equivalents are considered to be within the scope of this invention and are covered by the following claims.

We claim:

1. A method for suppressing the immune response of a recipient mammal to a donor hematopoietic stem cell graft comprising expressing a recombinant FasL gene in a donor hematopoietic stem cell of the donor graft, wherein expression of the recombinant FasL gene in the donor hematopoietic stem cell of the donor graft suppresses the immune response of the recipient mammal to the donor graft.

2. The method of claim 1, wherein the expression of the recombinant FasL gene in the donor graft cell results in the selective deletion of developing anti-donor T cells of the recipient mammal.

3. The method of claim 1 or 2, wherein the donor hematopoietic stem cell graft is an allogeneic graft.

4. The method of claim 1 or 2, wherein the donor hematopoietic stem cell graft is a xenogeneic graft.

5. The method of claim 1 or 2, wherein expression of the recombinant FasL gene in the donor hematopoietic cell is effected by transducing the donor hematopoietic cell with a FasL gene expression vector.

6. The method of claim 5, wherein the FasL gene expression vector comprises a nucleotide sequence that hybridizes under stringent conditions to the FasL nucleic acid sequence shown in FIG. 8A or its complement.

7. The method of claim 5, wherein the FasL gene expression vector comprises a nucleotide sequence that encodes the polypeptide sequence shown in FIG. 8B.

8. The method of claim 5, wherein the FasL gene expression vector encodes a non-cleavable form of FasL.

9. The method of claim 8, wherein the non-cleavable form of FasL is shown in FIG. 9B.

10. The method claim 8, wherein the non-cleavable form of FasL is a deletion mutant.

11. The method of claim 1 or 2, wherein the recombinant FasL gene is expressed from a donor hematopoietic cell chromosome.

12. The method of claim 1 or 2, wherein the recombinant FasL gene is expressed from a retroviral expression vector.

13. The method of claim 1 or 2, wherein the recombinant FasL gene is expressed from a lentiviral expression vector.

14. The method of claim 1 or 2, further comprising providing a donor dendritic cell expressing a recombinant FasL gene.

15. The method of claim 14, wherein expression of the recombinant FasL gene in the donor dendritic cell is effected by transducing the donor graft cell with a FasL gene expression vector.

16. The method of claim 15, wherein the FasL gene expression vector comprises a nucleotide sequence that hybridizes under stringent conditions to the FasL nucleic acid sequence shown in FIG. 8A or its complement.

17. The method of claim 15, wherein the FasL gene expression vector comprises a nucleotide sequence that encodes the polypeptide sequence shown in FIG. 8B.

18. The method of claim 15, wherein the FasL gene expression vector encodes a non-cleavable form of FasL.

19. The method of claim 18, wherein the non-cleavable form of FasL is shown in FIG. 9B.

20. The method claim 18, wherein the non-cleavable form of FasL is a deletion mutant.

21. The method of claim 15, wherein the recombinant FasL gene is expressed from a retroviral expression vector.

22. The method of claim 15, wherein the recombinant FasL gene is expressed from a lentiviral expression vector.

23. The method of claim 1 or 2, further comprising providing means for inhibiting a FasL dependent intracellular signal in the hematopoietic stem cell expressing the recombinant FasL gene.

24. The method of claim 23, wherein the means for inhibiting the FasL dependent intracellular signal is provided by a vector encoding a dominant negative form of FADD.

25. The method of claim 24, wherein the dominant negative form of FADD is a deletion mutant.

26. A recombinant donor hematopoietic stem cell comprising a recombinant FasL gene, wherein the recombinant FasL gene is capable of expressing a recombinant FasL polypeptide that suppresses the immune response of a recipient mammal to a donor graft.

27. A method for increasing tolerance to a solid organ allograft in a subject comprising:
providing a solid organ allograft from a donor organism to the subject; and further providing a recombinant hematopoietic cell graft expressing a recombinant FasL gene from said donor organism to said subject,
wherein the recombinant hematopoietic cell graft expressing the recombinant FasL gene from the donor organism increases tolerance to the donor solid organ allorgraft in the subject.

28. The method of claim 27, wherein the hematopoietic cell graft comprises a recombinant donor dendritic cell.

29. The method of claim 27, wherein the hematopoietic cell graft comprises a recombinant hematopoietic stem cell.

30. The method of claim 27, wherein the donor solid organ allograft is a cardiac allograft.

31. The method of claim 27, further comprising treating the subject with an agent that promotes a co-stimulatory blockade.

32. The method of claim 27, wherein the agent that promotes a co-stimulatory blockade is selected from the group consisting of: an anti-CD40 antibody and CTLA4-Ig.

33. A method for suppressing a graft versus host immune response in a host organism receiving a donor allograft comprising providing to the host a host hematopoietic cell graft expressing a recombinant FasL gene.

34. The method of claim 33, wherein the recombinant host hematopoietic cell is a hematopoietic stem cell.

35. The method of claim 34, wherein the recombinant host hematopoietic cell is a dendritic cell.
